(19) United States
(12) Patent Application Publication
(10) Pub. No.: US 2009/0177094 A1
(43) Pub. Date: Jul. 9, 2009

(54) SYSTEMS AND METHODS FOR TISSUE EXAMINATION, DIAGNOSTIC, TREATMENT, AND/OR MONITORING

(75) Inventors: William J. Brown, Durham, NC (US); Adam Wax, Chapel Hill, NC (US)

Correspondence Address: WITHROW & TERRANOVA, P.L.L.C., 100 REGENCY FOREST DRIVE, SUITE 160, CARY, NC, 27518, US

(73) Assignee: Oncoscope, Inc., Durham, NC (US)

(21) Appl. No.: 12/350,689

(22) Filed: Jan. 8, 2009

Related U.S. Application Data
(60) Provisional application No. 61/019,662, filed on Jan. 8, 2008.

(51) Int. Cl. A61B 6/00; A61B 1/06; A61B 18/14; A61B 18/18; A61B 18/04

(52) U.S. Cl. 600/476; 600/160; 606/2; 606/20; 606/32

(57) ABSTRACT

Procedures, techniques, and systems for in vivo monitoring, diagnosis, and treatment of tissue during the same or concomitant medical procedure. In disclosed embodiments, during a same or concomitant procedure or examination, tissue can be scanned on a localized level using a real-time optical biopsy system. The real-time optical biopsy system may involve angle-resolved and/or Fourier domain low coherence interferometry (LCI). Because the scanning can be performed in real-time, diagnosis can also be performed in real-time and during the same or concomitant medical procedure. As a result, therapy, if needed, can also be administered to the tissue during the same or concomitant medical procedure. Monitoring of the tissue after therapy can be performed during the same or subsequent procedure. Thus, the procedures and techniques disclosed herein allow detection of tissue anomalies during a first procedure on the patient without waiting for untimely biopsy results, thus providing earlier anomaly detection and treatment and potentially better and timely results and at a lower cost.

### RELATED APPLICATIONS

This patent application claims priority to U.S. Provisional Patent Application Ser. No. 61/019,662, filed on Jan. 8, 2008 and entitled “Systems and Methods for Tissue Diagnostic, Monitoring, and/or Therapy,” which is incorporated herein by reference in its entirety.

This patent application is related to U.S. Pat. No. 7,102,758, filed on May 6, 2003 and entitled “Fourier Domain Low-Coherence Interferometry for Light Scattering Spectroscopy Apparatus and Method,” which is incorporated herein by reference in its entirety.

This patent application is also related to U.S. patent application Ser. No. 11/548,468, filed on Oct. 11, 2006 and entitled “Systems and Methods for Endoscopic Angle-Resolved Low Coherence Interferometry,” which is incorporated herein by reference in its entirety.

This patent application is also related to U.S. patent application Ser. No. 12/210,620, filed on Sep. 15, 2008 and entitled “Apparatuses, Systems, and Methods for Low-Coherence Interferometry (LCI),” which is incorporated herein by reference in its entirety.

This patent application is also related to U.S. patent application Ser. No. 11/780,879, filed on Jul. 20, 2007 and entitled “Protective Probe Tip, Particularly for Use on a Fiber-Optic Probe Used in an Endoscopic Application,” which is incorporated herein by reference in its entirety.

### FIELD OF THE DISCLOSURE

The disclosure is related to diagnosing and monitoring tissue using optical biopsy, and treating tissue in vivo, without extracting the tissue for biopsy.

### BACKGROUND

Up to eight-five percent of all human cancers start in the epithelial tissue. As shown in Table 1 below, some of these cancers, such as melanoma of the skin for example, are easier to detect and to treat, resulting in better five-year survival rates, although there is still need for improved detection and treatments. Others, particularly in the esophagus, colon, and lung are difficult to find at an early stage, have low survival rates if found early, and have extremely low survival rates if found at later stages. Furthermore, some patient populations have a higher risk of cancer occurrence based on other factors.

TABLE 1
Cancer Diagnoses, Death, and Survival Rates

| | | | | | | Melanoma of |
|---|---|---|---|---|---|---|
| | Esophagus | Colon | Lung | Cervix | Bladder | the Skin |
| Diagnoses, Deaths and | | | | | | |
| Survival Rate | | | | | | |
| New Diagnoses 2006 | 14,550 | 148,610 | 174,470 | 9,710 | 61,420 | 62,190 |
| Deaths 2006 | 13,770 | 55,170 | 162,460 | 3,700 | 13,060 | 7,910 |
| 5 Year Survival Rate | 15.6% | 64.1% | 15.0% | 71.6% | 80.8% | 91.5% |
| Stage of Cancer When | | | | | | |
| Diagnosed | | | | | | |
| Confined | 24% | 39% | 16% | 52% | 74% | 80% |
| Regional | 29% | 37% | 37% | 34% | 19% | 12% |
| Metastasized | 30% | 19% | 39% | 9% | 4% | 4% |
| Unknown | 17% | 5% | 8% | 5% | 3% | 4% |
| 5 Year Survival Rate Based | | | | | | |
| on Stage at Diagnosis | | | | | | |
| 5 Year Survival - Confined | 33.6% | 90.4% | 49.3% | 92.0% | 93.7% | 99.0% |
| 5 Year Survival - Regional | 16.8% | 68.1% | 15.5% | 55.5% | 46.0% | 64.9% |
| 5 Year Survival - Metastasized | 2.6% | 9.8% | 2.1% | 14.6% | 6.2% | 15.3% |
| 5 Year Survival - Unknown | 10.8% | 34.6% | 7.9% | 59.1% | 60.4% | 76.8% |

In general, the course of care for most cancers involves a procedure to acquire data (typically tissue). The acquired tissue is typically sent off to a laboratory outside of the context of the tissue acquisition procedure. Depending on the circumstances, this analysis may take several hours, days, or weeks. After the analysis is returned, the physician may make a diagnosis, and if treatment is necessary, a treatment procedure may be employed. Because of the time required for analysis of the acquired tissue, the treatment procedure is performed during a separate patient procedure or examination, and typically during a patient visit days to weeks later. Treatment may then be repeated at various time points subsequent during separate patient procedures to verify that the cancer has been eliminated and has not returned. As one example, a dermatologist may visually inspect the skin. If a suspicious mole is found, a piece of tissue may be cut out and sent to a pathology lab for analysis. Based on the pathology information, the patient may undergo a Moh's surgery on the mole where successive layers of tissue are sliced off and sent for immediate pathology analysis until a layer with no cancer cells is obtained. The patient will probably undergo follow-up visits to visually inspect that spot and verify that the cancer has not returned. Similar procedures will be followed for other cancers, but with the disadvantage that is it difficult to accurately track the tissue location when inside the body in places such as the colon, esophagus, bladder, cervix, oral cavity, and others.

As another example, patients with Gastroesophageal Reflux Disease (GERD) may progress to Barrett's Esophagus (BE), at which point they have a 30 to 150 times greater chance of getting esophageal cancer than the general population. As a result, the current standard of care is for these patients to undergo a random biopsy surveillance procedure on a periodic basis. The biopsy procedure consists of a four-quadrant biopsy taken every centimeter through the affected portion of the esophagus (the Seattle Protocol). These biopsies are sent to a pathology lab and, based on the results, the patient comes back for the next round of surveillance or further treatment occurs such as an oral drug, or in cases of high grade dysplasia or cancer, an esophagectomy.

There are significant issues with this current approach to detection and treatment of numerous cancer types including lack of coverage of tissue, lack of sufficient detection at early stages of the disease, time lag between sample acquisition and treatment procedures due to the inability to acquire and diagnose tissue quickly during the same procedure or patient examination, and need for multiple procedures. Because the diagnosis occurs later in time after the tissue acquisition, it is also difficult to return to the exact location of the biopsy for further monitoring and treatment. Misdiagnosis by the pathologist, and lack of effective treatment options can occur as a result.

Advances by the applicant in low coherence interferometry (LCI), including angle-resolved LCI (a/LCI) and Fourier domain LCI (f/LCI) (referred to collectively as “f/a/LCI”) enable in vivo diagnosis of epithelial tissue health, specifically if tissue is normal, pre-cancerous, cancerous, diseased, or abnormal. This opens up new opportunities, the most significant described of which in the invention to follow is the potential to diagnosis, treat, and monitor tissue in vivo, employing methods, processes, techniques, and systems that use real-time optical biopsy systems, including f/a/LCI systems, for examining and monitoring tissue during the course of the same or concomitant medical procedure to determine if a therapeutic should be applied to the tissue.

### SUMMARY OF THE DETAILED DESCRIPTION

Embodiments in the detailed description cover methods, processes, techniques, and systems that use real-time optical biopsy systems for examining and monitoring tissue during the course of the same or concomitant medical procedure to determine if a therapeutic should be applied to the tissue. The real-time optical biopsy systems disclosed herein are systems based on low coherence interferometer (LCI) detection of light scattered from a sample that can obtain structural and/or depth-resolved information regarding in vivo tissue in a single data collection event and which permits diagnosis in connection with the data collection. New therapeutic procedures and techniques can be implemented as a result. Specifically, tissue can be diagnosed and treated during the same or concomitant medical procedure or examination. This is an improvement over traditional biopsy techniques where diagnosis of the tissue cannot be performed until the biopsy procedure is completed and the biopsy results are received after the procedure thereby delaying treatment. Further, the location of the analyzed tissue is known thereby allowing localized treatment of the tissue, or the location may be returned to for follow up monitoring.

These methods, processes, techniques, and systems disclosed herein offer an opportunity to significantly improve the standard of care for patients and decrease overall health care costs by diagnosing and treating tissue conditions, including pre-cancerous and cancerous conditions, in vivo. The methods, processes, and techniques disclosed herein effectively reduce the treatment time to the time of a first medical procedure on the patient, thus providing earlier treatment and potentially better and more timely results at a lower cost. This also provides more accurate diagnosis and determination of treatment effectiveness since the monitoring is performed on a localized level with the ability to diagnose, treatment, and monitor the affected tissue during the same or concomitant medical procedure or examination. The above-described methods, processes, techniques, and systems also enable more efficient diagnosis, treatment, and monitoring, or throughput of patients. This may be particularly important where health facilities and appointments are a limited resource.

In disclosed embodiments, real-time optical biopsy systems include Fourier domain and/or angle-resolved low coherence interferometry (LCI) optical biopsy technologies (hereinafter referred to collectively and generically as “f/a/LCI”). During the same or concomitant medical procedure or examination, a physician or other health care professional will be able to scan tissue in vivo on a localized level using a real-time f/a/LCI system, monitor the scan, diagnose tissue status as normal, pre-cancerous, cancerous, abnormal, diseased or the like, and administer a therapeutic based on the tissue status, if desired or needed. Because the scan of the tissue can be performed in real-time using the real-time f/a/LCI system, which collects depth-resolved and/or structural information in a single data collection event, monitoring of the treated tissue can also be performed in real-time and during the same or concomitant medical procedure or tissue examination. In the same regard, diagnosis of the tissue can also be performed during the same or concomitant medical procedure or tissue examination. A therapeutic can also be administered during the same or concomitant procedure or tissue examination. If desired, multiple medical procedures at different time points can then be used to monitor the status of tissue in vivo over time to determine tissue status, health or response to treatment. This allows physicians or other clinicians to fully maximize the information opportunity provided by the real-time f/a/LCI system and vastly improve the quality of care for the patient.

In one embodiment, a method for examining and monitoring tissue to determine if a therapeutic should be applied to the tissue during a same or concomitant medical procedure is provided. The method includes optically examining using a real time f/a/LCI system a tissue to detect tissues that are cancerous, abnormal, diseased or the like which conditions are generally not perceptible to the human eye. Real-time feedback information is monitored regarding the examination of the tissue from the real-time f/a/LCI system. Based on the real-time feedback information, a diagnosis is made as to whether a treatment should be applied to the tissue. If a treatment is to be applied, a selected therapy or combination of therapies is applied during the same or concomitant medical procedure.

The new methods, processes, techniques, and systems address the shortcoming of the current approaches. For example, since real-time optical biopsy systems can acquire data points in short periods of time (e.g., in a few seconds or minutes), it is possible to scan much larger areas of the tissue during a same or concomitant medical procedure. Furthermore, real-time f/a//LCI systems can detect tissue changes at an earlier stage in the disease. A therapeutic can be delivered immediately to a localized area where the real-time f/a/LCI system detected pre-cancerous, cancerous, abnormal, diseased tissue, or to a general area during the same or concomitant medical procedure. Subsequent scans can be taken to verify the treatment outcome and monitor tissue health over time. Information from the real-time optical biopsy systems described herein can be used to determine dosing levels or which choice of multiple treatment options to use. A standardized database in the computer can be employed to allow consistent analysis of tissue based on a database of tissue characteristics versus tissue health by detecting anomalies in tissue which may be pre-cancerous, cancerous, abnormal, diseased or the like.

Some implementations include the integration of a real-time optical biopsy system with an endoscope and/or therapeutic system. This integration results in a system with the capability to both diagnose and treat tissue in vivo. Several architectures are described including the use of an endoscopic probe, where a real-time optical biopsy system probe and the endoscopic light probe share or occupy one or more channels. Several architectures are also described including the use of multi-channel endoscopes where the real-time optical biopsy system probe occupies one channel and a therapeutic applicator can occupy another channel. The therapeutic system may be manually controlled or computer-controlled. There are a wide range of possible therapeutics including, but not limited to, elements, compounds, drugs, liquids, heat, cold, radio-frequency (RF) ablation, photodynamic therapy, and radiation. Another architecture example uses a single channel endoscope where the real-time optical biopsy system probe and the therapeutic system occupy the same fiber or fiber bundle channel. Yet another implementation uses a scanning real-time optical biopsy system where multiple points are scanned in an automated or semi-automated fashion.

In addition to clinical activities, a real time optical biopsy such as f/a/LCI can be used in research activities, particularly those that track tissue health over time, such as in the study of chemo-preventatives. Real time f/a/LCI could be used to scan a tissue sample or cell culture at various points in time to assess changes in the status of the tissue or cells. For example a cell culture of cancer cells could be scanned and then treated with a chemo-preventative and then scanned at subsequent time points to see if the cancer cells were killed (such as by apoptosis) or not.

### BRIEF DESCRIPTION OF THE DRAWINGS

FIG. 1 is a flowchart of an exemplary diagnosis, treatment, and monitoring process according to an embodiment;

FIG. 2 is a diagram of an exemplary endoscope;

FIG. 3 is a diagram of an exemplary real-time f/a/LCI system employed in an instrument channel of an endoscope for determining tissue status in vivo;

FIG. 4A is a schematic of one exemplary embodiment of the real-time f/a/LCI system employing a Mach-Zehnder interferometer;

FIG. 4B is an illustration showing the relationship of the detected scattering angle to a slit of spectrograph in the interferometer arrangement of FIG. 4A;

FIG. 5 is a flowchart illustrating exemplary steps performed by an interferometer apparatus to recover depth-resolved spatial cross-correlated information about the sample for analysis;

FIGS. 6A-D illustrate examples of f/a/LCI data recovered in the spectral domain for an exemplary sample of polystyrene beads, comprising the total acquired signal (FIG. 6A), the reference field intensity (FIG. 6B), the signal field intensity (FIG. 6C), and the extracted, cross-correlated signal between the reference and signal field intensities (FIG. 6D);

FIG. 7A is an illustration of an axial spatial cross-correlated function performed on the cross-correlated f/a/LCI data illustrated in FIG. 6D as a function of depth and angle;

FIG. 7B is an illustration of an angular distribution plot of raw and filtered data regarding scattered sample signal intensity as a function of angle in order to recover size information about the sample;

FIG. 8A is an illustration of the filtered angular distribution of the scattered sample signal intensity compared to the best fit Mie theory to determine size information about the sample;

FIG. 8B is a Chi-squared minimization of size information about the sample to estimate the diameter of cells in the sample;

FIG. 9 is a schematic of an exemplary embodiment of a real-time f/a/LCI system employing an optical fiber probe;

FIG. 10A is a cutaway view of an f/a/LCI fiber-optic probe tip that may be employed by the real-time f/a/LCI system of FIG. 9;

FIG. 10B illustrates the location of the fiber probe in the real-time f/a/LCI system of FIG. 10A;

FIG. 11A is an illustration of an alternative fiber-optic real-time f/a/LCI system;

FIG. 11B is an illustration of sample illumination and scattered light collection with the distal end of probe in the real-time f/a/LCI system of FIG. 11A;

FIG. 11C is an illustration of an image of the illuminated distal end of the probe of the real-time f/a/LCI system illustrated in FIG. 11A;

FIGS. 12A and 12B are diagrams of an exemplary real-time f/a/LCI system and endoscope, wherein the real-time f/a/LCI system is employed in an instrument channel of an endoscope, and a therapeutic delivery system is employed in a second endoscope channel;

FIG. 13 is a diagram of an exemplary real-time f/a/LCI system and endoscope, wherein the real-time f/a/LCI system is employed in an instrument channel of an endoscope, and a radio-frequency (RF) ablation therapy system is employed in a second channel of the endoscope;

FIG. 14 is a diagram of an exemplary real-time f/a/LCI system and endoscope, wherein the real-time f/a/LCI system is employed in an instrument channel of an endoscope, and a photodynamic therapy system is employed in a second channel of the endoscope;

FIGS. 15A and 15B are diagrams of an exemplary real-time f/a/LCI system and endoscope, wherein the real-time f/a/LCI system is employed in an instrument channel of an endoscope, and a substance dispenser is employed in a second channel of the endoscope;

FIGS. 16A and 16B are diagrams of an exemplary real-time f/a/LCI system and endoscope, wherein the real-time f/a/LCI system is employed in an instrument channel of an endoscope, and a hot/cold therapeutic system is employed in a second channel of the endoscope;

FIG. 17 is a diagram of an exemplary real-time f/a/LCI system and endoscope, wherein the real-time f/a/LCI system is employed in an instrument channel of an endoscope, and a surgical instrument(s) for tissue removal is employed in a second channel of the endoscope;

FIGS. 18A and 18B are diagrams of an exemplary fiber optic real-time f/a/LCI system integrated into a single channel endoscope, wherein the fiber optic real-time f/a/LCI system and a light therapy system share an optical channel in the endoscope;

FIG. 19 is a diagram of an exemplary real-time f/a/LCI system employed in an instrument channel of an endoscope with a separate therapeutic system;

FIG. 20 is a diagram of an exemplary scanning real-time f/a/LCI system employed in an instrument channel of an endoscope with a therapeutic system employed in a second channel of the endoscope;

FIG. 21 is a diagram of an exemplary real-time f/a/LCI system with scanner control and an integrated computer employed in an instrument channel of an endoscope with a disposable probe tip;

FIG. 22 is a table that summarizes possible combinations of LCI systems and endoscopes for monitoring tissue and types of therapeutics for treating monitored tissue;

FIG. 23 is an illustration of a cutaway view of an exemplary probe tip employing a fixed sheath;

FIG. 24 is an illustration of a solid view the probe tip illustrated in FIG. 23;

FIG. 25A is an illustration of a cutaway view of an exemplary probe tip employing a removable sheath;

FIG. 25B is an illustration of the probe tip illustrated in FIG. 25A, and employing an angled optical window;

FIG. 26 is an alternative illustration of a solid view of the probe tip illustrated in FIG. 25A;

FIG. 27 is an illustration of the probe tip illustrated in FIGS. 25A and 26, employing an optional sterile skirt;

FIG. 28 is an illustration of the probe tip illustrated in FIG. 27, with the sterile skirt deployed;

FIG. 29 is an illustration of the probe tip illustrated in FIG. 27, further employing a vacuum-assisted suction device to facilitate application of the probe tip to a tissue surface;

FIG. 30A is a diagram of an exemplary embodiment of an f/LCI system;

FIG. 31 is a diagram of another exemplary embodiment of an f/LCI system using fiber optic coupling;

FIGS. 32A and 32B are diagrams illustrating exemplary properties of a white light source;

FIGS. 33A and 33B are diagrams of an exemplary axial spatial cross-correlation function for a coverslip sample;

FIGS. 34A and 34B are diagrams of exemplary spectra obtained for front and back surfaces of a coverglass sample when no microspheres are present;

FIGS. 35A and 35B are diagrams of exemplary spectra obtained for front and back surfaces of a coverglass sample when microspheres are present;

FIGS. 36A and 36B are diagrams of exemplary ratios of spectra in FIGS. 33A and 33B, and FIGS. 34A and 34B illustrating scattering efficiency of spheres for front and back surface reflections;

FIG. 37 is a diagram of a generalized version of the system shown in FIGS. 30 and 31;

FIG. 38 is a block diagram of an exemplary embodiment of a tissue monitoring method using an f/LCI system;

FIG. 39 is a block diagram of another exemplary embodiment of a tissue monitoring method using an f/LCI system;

FIG. 40 is a schematic diagram of an exemplary swept-source (SS) angle-resolved low-coherence interferometry (LCI) (SS a/LCI) apparatus and system that is used to detect information about a sample of interest;

FIG. 41 is a schematic diagram illustrating the angular light directed to the sample and detection of the angular scattered light returned from the sample using the SS a/LCI system illustrated in FIG. 40;

FIG. 42 is a flowchart illustrating an exemplary process for detecting spatially and depth-resolved information about the sample using the exemplary SS a/LCI apparatus and system of FIGS. 40 and 41;

FIG. 43A is a schematic diagram of an exemplary fiber optic-based swept-source (SS) angle-resolved low-coherence interferometry (LCI) (SS a/LCI) apparatus and system that is used to detect information about a sample of interest;

FIG. 43B is another schematic diagram of the exemplary fiber optic-based swept-source (SS) angle-resolved low-coherence interferometry (LCI) (SS a/LCI) apparatus and system of FIG. 43A;

FIG. 44 is a schematic diagram of an exemplary swept-source multiple angle SS a/LCI (MA SS a/LCI) apparatus and system that is used to detect information about a sample of interest;

FIG. 45 is a schematic diagram illustrating the angular light directed to the sample and detection of the angularly distributed scattered light returned from the sample in two dimensions using the MA SS a/LCI system illustrated in FIG. 44;

FIG. 46 is an exemplary model of a two-dimensional image of a diffraction pattern from a sample acquired using the MA SS a/LCI system of FIG. 44;

FIG. 47 is a schematic diagram of an exemplary optic fiber breakout from a fiber optic cable employed in the MA SS a/LCI apparatus and system of FIG. 44;

FIG. 48 is a schematic diagram of relative fiber positions of an endoscopic fiber optic detection device that can be employed in the MA SS a/LCI apparatus and system of FIG. 44;

FIG. 49 is a schematic diagram of a multiple channel time domain a/LCI apparatus and system that is used to detect information about a sample of interest;

FIG. 50 is a schematic diagram of an alternative multiple channel time domain a/LCI apparatus and system that is used to detect information about a sample of interest;

FIG. 51 is a schematic diagram of an alternative time domain a/LCI apparatus and system that collects angular information about the sample in serial fashion, but collects depth information using Fourier domain techniques;

FIG. 52 is a schematic diagram of a fiber optic-based time domain a/LCI apparatus and system that collects angular information about the sample in serial fashion, but collects depth information using Fourier domain techniques;

FIG. 53 is a schematic diagram of a multi-spectral a/LCI apparatus and system; and

FIG. 54 is a schematic diagram of a fiber optic-based multi-spectral a/LCI apparatus and system.

### DETAILED DESCRIPTION

The embodiments set forth below represent the necessary information to enable those skilled in the art to practice the invention and illustrate the best mode of practicing the invention. Upon reading the following description in light of the accompanying drawing figures, those skilled in the art will understand the concepts of the invention and will recognize applications of these concepts not particularly addressed herein. It should be understood that these concepts and applications fall within the scope of the disclosure and the accompanying claims.

Embodiments in the detailed description cover methods, processes, techniques, and systems that use real-time optical biopsy systems for examining and monitoring tissue during the course of the same or concomitant medical procedure to determine if a therapeutic should be applied to the tissue. The real-time optical biopsy systems disclosed herein are systems based on low coherence interferometer (LCI) detection of light scattered from a sample that can obtain structural and/or depth-resolved information regarding in vivo tissue in a single data collection event and which permits diagnosis in connection with the data collection. New therapeutic procedures and techniques can be implemented as a result. Specifically, tissue can be diagnosed and treated during the same or concomitant medical procedure or examination. This is an improvement over traditional biopsy techniques where diagnosis of the tissue cannot be performed until the biopsy procedure is completed and the biopsy results are received after the procedure thereby delaying treatment. Further, the location of the analyzed tissue is known thereby allowing localized treatment of the tissue, or the location may be returned to for follow up monitoring.

These methods, processes, techniques, and systems disclosed herein offer an opportunity to significantly improve the standard of care for patients and decrease overall health care costs by diagnosing and treating tissue conditions, including pre-cancerous and cancerous conditions, in vivo. The methods, processes, and techniques disclosed herein effectively reduce the treatment time to the time of a first medical procedure on the patient, thus providing earlier treatment and potentially better and more timely results at a lower cost. This also provides more accurate diagnosis and determination of treatment effectiveness since the monitoring is performed on a localized level with the ability to diagnose, treatment, and monitor the affected tissue during the same or concomitant medical procedure or examination. The above-described methods, processes, techniques, and systems also enable more efficient diagnosis, treatment, and monitoring, or throughput of patients. This may be particularly important where health facilities and appointments are a limited resource.

In disclosed embodiments, real-time optical biopsy systems include Fourier domain and/or angle-resolved low coherence interferometry (LCI) optical biopsy technologies (hereinafter referred to collectively and generically as “f/a/LCI”). During the same or concomitant medical procedure or examination, a physician or other health care professional will be able to scan tissue in vivo on a localized level using a real-time f/a/LCI system, monitor the scan, diagnose tissue status as normal, pre-cancerous, cancerous, abnormal, diseased or the like, and administer a therapeutic based on the tissue status, if desired or needed. Because the scan of the tissue can be performed in real-time using the real-time f/a/LCI system, which collects depth-resolved and/or structural information in a single data collection event, monitoring of the treated tissue can also be performed in real-time and during the same or concomitant medical procedure or tissue examination. In the same regard, diagnosis of the tissue can also be performed during the same or concomitant medical procedure or tissue examination. A therapeutic can also be administered during the same or concomitant procedure or tissue examination. If desired, multiple medical procedures at different time points can then be used to monitor the status of tissue in vivo over time to determine tissue status, health or response to treatment. This allows physicians or other clinicians to fully maximize the information opportunity provided by the real-time f/a/LCI system and vastly improve the quality of care for the patient.

The new methods, processes, techniques, and systems address the shortcoming of the current approaches. For example, since real-time optical biopsy systems can acquire data points in short periods of time (e.g., in a few seconds or minutes), it is possible to scan much larger areas of the tissue during a same or concomitant medical procedure. Furthermore, real-time f/a//LCI systems can detect tissue changes at an earlier stage in the disease. A therapeutic can be delivered immediately to a localized area where the real-time f/a/LCI system detected pre-cancerous, cancerous, abnormal, diseased tissue, or to a general area during the same or concomitant medical procedure. Subsequent scans can be taken to verify the treatment outcome and monitor tissue health over time. Information from the real-time optical biopsy systems described herein can be used to determine dosing levels or which choice of multiple treatment options to use. A standardized database in the computer can be employed to allow consistent analysis of tissue based on a database of tissue characteristics versus tissue health by detecting anomalies in tissue which may be pre-cancerous, cancerous, abnormal, diseased or the like.

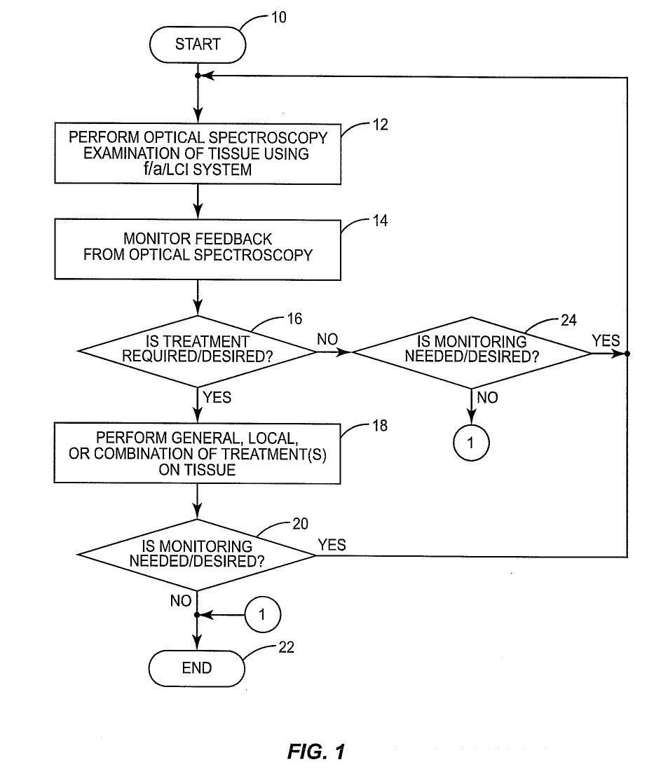

FIG. 1 illustrates an overall exemplary flowchart of new methods, processes and techniques that are made possible by this disclosure, especially because of the ability of real-time optical biopsy systems to detect abnormal tissues quickly on a localized level. Any or all of these steps can be provided or performed. As illustrated in FIG. 1, an exemplary process starts (block 10) and an in vivo examination of tissue using a real-time optical biopsy system is performed (block 12). Real-time optical biopsy systems are optical biopsy systems that can examine and monitor tissue during the course of the same or concomitant medical procedure to determine if a therapeutic should be applied to the tissue. In this example, an f/a/LCI real-time optical biopsy, examples of which are described in more detail in this application, is employed to perform an in vivo examination of tissue (block 12). As will be discussed in more detail below, a real-time f/a/LCI system allows obtaining of information about tissue of interest quickly, typically on the order of seconds or minutes. For example, the real-time f/a/LCI system may allow obtaining of information about tissue of interest in one second or less.

Because of timely acquisition of tissue information, real-time feedback information regarding the tissue is provided by the real-time f/a/LCI system and can be monitored by a physician or clinician in real-time and during the same or concomitant medical procedure or examination, thereby minimizing time, inconvenience, and/or discomfort to the patient (block 14). Further, a timely diagnosis of the results can be performed. A diagnosis of the tissue information from the real-time f/a/LCI system can be performed to determine if treatment of the examined tissue is necessary or desired. If necessary or desired, the treatment can be undertaken during the same or concomitant medical procedure or examination, and without having to wait for biopsy results or only after lengthy scans are performed (block 16). If treatment is required, a general, local, or combination of general and local treatment can be performed on the tissue in the same localized area of examined by the real-time f/a/LCI system with accuracy and during the same or concomitant medical procedure or examination of the patient (block 18).

Thereafter, it can be determined if further monitoring of the affected tissue is desired or needed (block 20). This further monitoring can be performed during the same or concomitant medical procedure or examination of the patient or during a subsequent medical procedure or examination of the patient. If further monitoring is needed, the overall process can be performed again (block 10) wherein an optical biopsy of the treated tissue can be performed (block 12). If further monitoring is not required, or it is not required or possible to see results during the same or concomitant medical procedure or examination of the patient, the process ends (block 22). Likewise, if no treatment is desired or needed (block 16), and further monitoring is not required or desired (block 24), the process ends (block 22). If further monitoring is required even though treatment is not required or desired after an optical biopsy (block 24), the process can be repeated (block 10) and another optical biopsy performed (block 12).

In this regard, the above-described methods and processes can reduce the number of medical procedures required to achieve a therapeutic result. If a traditional biopsy is performed, a diagnosis of the tissue cannot be performed until the biopsy results are received. Therapy, if needed or desired, can only be performed during a subsequent medical procedure or examination of the patient. The above-described methods and processes also allow monitoring of the effectiveness of the therapy during the same or concomitant medical procedure if desired, because the information regarding the tissue can be obtained and analyzed during the same or concomitant medical procedure and after therapy has been administered. This effectively reduces the application of treatment to the time of a first medical procedure on the patient, thus providing earlier treatment and potentially better and more timely results at a lower cost. This also provides more accurate diagnosis and determination of treatment effectiveness since the monitoring is performed on a localized level with the ability to diagnose, monitor, and treat the affected tissue during the same or concomitant medical procedure or examination. The above-described methods and processes also enable more efficient diagnosis, treatment, and monitoring, or throughput of patients. This may be particularly important where health facilities and appointments are a limited resource.

As an example, a tissue examination procedure may be an esophageal endoscopy performed on patients with risk of esophageal cancer (such as those with Barrett's Esophagus). In the prior method, a physical biopsy of the esophagus is taken and sent to a pathological laboratory for analysis. It may take one week or so for a laboratory technician to analyze the extracted tissue sample and provide the information regarding the results to the attending physician. If, for example, it is determined that dysplasia is present, the patient is then scheduled for another medical procedure or examination in the future. An esophageal endoscopy is then performed again where a radio frequency (RF) ablation or other treatment may be performed. The monitoring of the treatment cannot be determined during the second medical procedure either. A biopsy must be performed in yet a subsequent medical procedure or examination, and the process repeated, thus adding substantial delay between the patient's first procedure of a biopsy and analysis of the effectiveness of the treatment.

With the methods, processes, techniques, and systems disclosed herein, the physician uses real-time f/a/LCI to scan tissue. Because the information regarding the scan is provided on a localized level and in real-time, the physician can treat any precancerous, cancerous, diseased, or abnormal areas concomitantly with the scanning. Alternately, the physician might first scan the tissue and then go back and ablate any areas of concern during the same or concomitant medical procedure. With the embodiments disclosed herein, there is the possibility of scanning, diagnosis and treatment in the same or concomitant medical procedure. Follow up might then consist of repeating this procedure at certain time intervals with additional treatment as necessary.

The remainder of this section focuses on system designs that allow these new methods, processes and techniques to be carried out in the process of examining and treating patients. Additional embodiments of the methods, processes and techniques disclosed include medical procedures using real-time f/a/LCI, examples of which are described in more detail below. Various systems may be implemented and used to carry out the methods, processes and techniques. Examples of these new systems and methods, processes, and techniques are described below in more detail in this application. These systems are not an exhaustive list, but illustrate examples enabled by the present invention to diagnose, monitor, and treat cancer using f/LCI, a/LCI, or f/a/LCI.

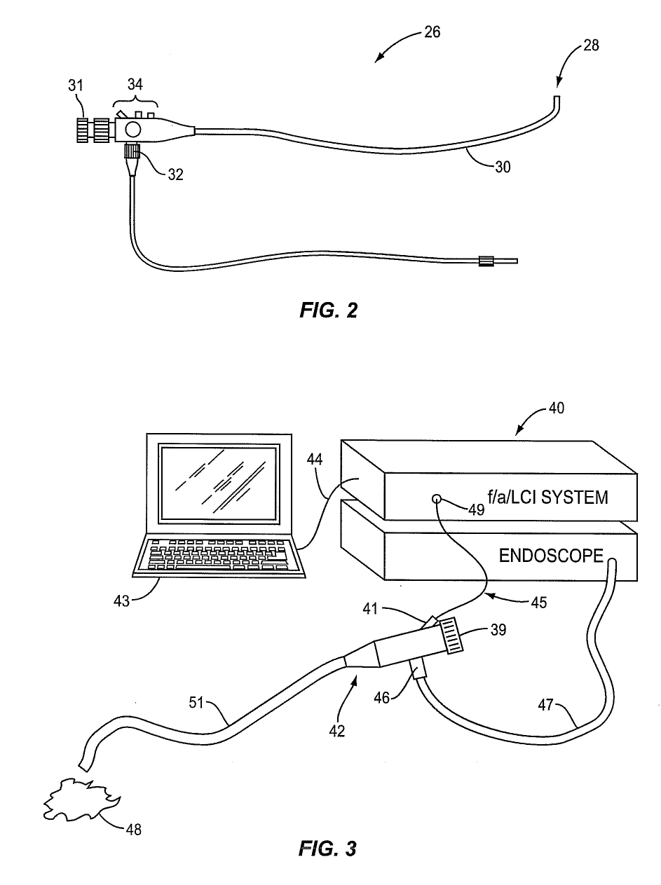

In one embodiment, the system that can be employed to carry out the medical procedure or examination can consist of: (1) a real-time f/a/LCI optical biopsy tissue diagnosis system, (2) an endoscope, and (3) a therapeutic that can be delivered via the endoscope. This integrated system will then allow the operator to assess the tissue health and apply the appropriate therapeutic to tissue meeting certain criteria. A typical biopsy endoscope 26 is illustrated in FIG. 2. The endoscope 26 may have a camera, aperture, or other imaging device 28 on the end of a shaft 30, which may be rigid or flexible, for visual inspection of tissue. An eyepiece 31 is used to review the images of the tissue captured by the aperture or imaging device 28. The endoscope 26 may have one or more channels 32 for introducing light and zero, and one or more instrument or accessory channels 34. As an example, a biopsy endoscope may have three channels, an integrated channel for visual inspection, an instrument channel through which biopsy forceps may be passed, and an instrument channel through which an f/a/LCI probe may be passed. There may also be channels for air and water, and endoscopes may have visual illumination sources at the distal end.

FIG. 3 illustrates an example of a real-time f/a/LCI system 40 employed in an instrument channel 41 of an endoscope 42 to perform optical biopsy of tissue during a patient procedure or examination, and which may be employed in the above-described methods, processes and techniques. This configuration may be useful in that an endoscope enables guided biopsy where the integrated real-time f/a/LCI system allows the operator to determine tissue status in vivo and use that information to collect biopsies from the areas of higher concern. As illustrated in FIG. 3, the real-time f/a/LCI system 40 is provided and interfaces with a computer 43 to control the operation of and receive data from the f/a/LCI system 40 regarding the tissue examined. In this regard, the computer 43 is interfaced with the real-time f/a/LCI system 40 via a communication line(s) 44. A fiber bundle or fiber probe 45 from a fiber port 49 on the real-time f/a/LCI system 40 is passed down the instrument channel 41 of the endoscope 42 to direct light to the tissue of interest and to collect depth-resolved angular distributions of scattered light from the tissue for diagnosis, as well be discussed in more detail below. A second instrument channel 46 can be provided on the endoscope 42 for receiving light, air, water, or other substance via a shaft 47 to assist in the examination of tissue 48. The physician can examine or monitor the tissue using the eyepiece 39 of the endoscope 42 as the real-time f/a/LCI system 40 scans the tissue 48 of interest. A shaft 51 of the endoscope 42 can be moved within the patient to examine the tissue 48 of interest.

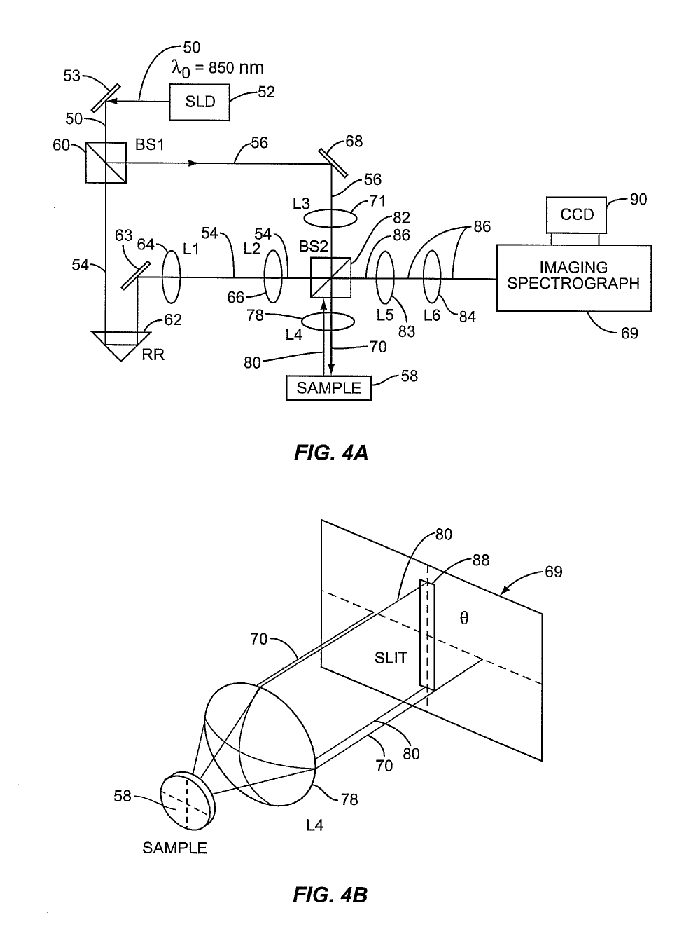

Before discussing various embodiments of real-time f/a/LCI systems and endoscope systems that may be used to examine, diagnose, and administer treatment to a patient's tissue, more information regarding real-time f/a/LCI systems is provided. FIGS. 4A-11C illustrate one possible real-time f/a/LCI system that may be employed to obtain, diagnose, and treat a patient's tissue during the same or concomitant medical procedure, and may also be employed to monitor the effectiveness of treatment during the same or subsequent procedures. In summary, the real-time f/a/LCI system illustrated in FIGS. 4A-11 in particular is called Fourier domain a/LCI (faLCI), which enables data acquisition at rapid rates using a single scan, sufficient to make in vivo applications feasible. The faLCI system can obtain angle-resolved and depth-resolved spectra information about a sample, in which depth and size information about the sample can be obtained with a single scan, and wherein the reference arm can remain fixed with respect to the sample due to only one scan required. A reference signal and a reflected sample signal are cross-correlated and dispersed at a multitude of reflected angles off of the sample, thereby representing reflections from a multitude of points on the sample at the same time in parallel. Other real-time Fourier domain and non Fourier domain LCI systems are described herein, which are collectively referred to as “f/a/LCI.”

Since this angle-resolved, cross-correlated signal is spectrally dispersed, the new data acquisition scheme is significant as it permits data to be obtained in seconds or minutes, a threshold determined to be necessary for acquiring data from in vivo tissues. Information about all depths of the sample at each of the multitude of different scattering angles on the sample can be obtained with one scan on the order of approximately 40 milliseconds. From the spatial, cross-correlated reference signal, structural (size) information can also be obtained using techniques that allow size information of scatterers to be obtained from angle-resolved data.

The faLCI technique in FIGS. 4A-11 uses the Fourier domain concept to acquire depth-resolved information. Signal-to-noise and commensurate reductions in data acquisition time are possible by recording the depth scan in the Fourier (or spectral) domain. The faLCI system combines the Fourier domain concept with the use of an imaging spectrograph to spectrally record the angular distribution in parallel. Thereafter, the depth-resolution of the present invention is achieved by Fourier transforming the spectrum of two mixed fields with the angle-resolved measurements obtained by locating the entrance slit of the imaging spectrograph in a Fourier transform plane to the sample. This converts the spectral information into depth-resolved information and the angular information into a transverse spatial distribution. The capabilities of faLCI have been initially demonstrated by extracting the size of polystyrene beads in a depth-resolved measurement.

The key advances of the present invention can be broken down into three components: (1) new rapid data acquisition methods, (2) fiber probe designs, and (3) data analysis schemes. Thus, the present invention is described in this matter for convenience in its understanding.

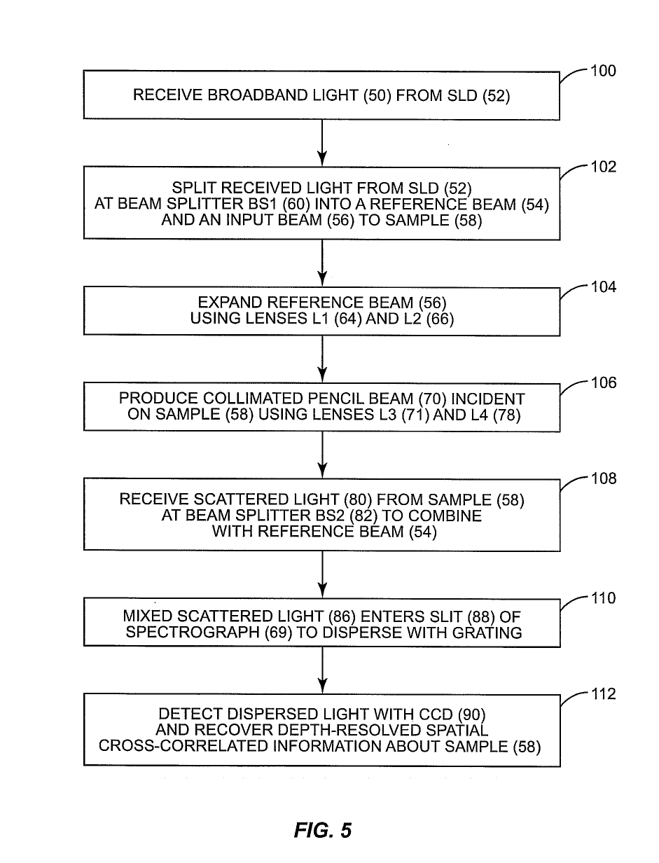

An exemplary apparatus, as well as the steps involved in the process of obtaining angle and depth-resolved distribution data scattered from a sample, are also set forth in FIG. 5. The faLCI scheme in accordance with one embodiment of the present invention is based on a modified Mach-Zehnder interferometer as illustrated in FIG. 4A. Broadband light 50 from a superluminescent diode (SLD) 52 is directed by a mirror 53 (step 100 in FIG. 5) and split into a reference beam 54 and an input beam 56 to a sample 58 by beamsplitter BS1 (60) (step 102 in FIG. 5). The output power of the SLD 52 may be 3 milliWatts, having a specification of λo=850 nm, Δλ=20 nm FWHM as an example, providing sufficiently low coherence length to isolate scattering from a cell layer within tissue. The path length of the reference beam 54 is set by adjusting retroreflector RR (62), but remains fixed during measurement. The reference beam 54 is expanded using lenses L1 (64) and L2 (66) to create illumination (step 104 in FIG. 5), which is uniform and collimated upon reaching a spectrograph slit 88 (FIG. 4B) in an imaging spectrograph 69. For example, L1 (64) may have a focal length of 1.5 centimeters, and L2 (66) may have focal length of 15 centimeters.

Lenses L3 (71) and L4 (78) are arranged to produce a collimated pencil beam 70 incident on the sample 48 (step 106 in FIG. 5). By displacing lens L4 (78) vertically relative to lens L3 (71), the collimated input beam 70 is made to strike the sample 58 at an angle of 0.10 radians relative to the optical axis in this example. This arrangement allows the full angular aperture of lens L4 (78) to be used to collect scattered light 80 from the sample 58. Lens L4 (78) may have a focal length of 3.5 centimeters as an example.

The light 80 scattered by the sample 58 is collected by lens L4 (78) and relayed by a 4f imaging system comprised of lenses L5 (83) and L6 (84) such that the Fourier plane of lens L4 (78) is reproduced in phase and amplitude at the spectrograph slit 88 (block 108 in FIG. 5). The scattered light 80 is mixed with the reference beam 54 at a second beamsplitter BS2 (82) (block 108 in FIG. 5) with the combined fields 86 falling upon the entrance slit 88 to the imaging spectrograph 69 (block 110 in FIG. 5). The imaging spectrograph 69 may be the model SP2150i, manufactured by Acton Research for example. FIG. 4B illustrates the distribution of scattering angle across the dimension of the spectrograph slit 88. The mixed scattered light 86 is dispersed with a high resolution grating (e.g., 1200 l/mm) and detected using a cooled charge-coupled device (CCD) 90 (e.g., 1340×400, 20 μm×20 μm pixels, Spec10:400, manufactured by Princeton Instruments) (block 112 in FIG. 5).

The mixed scattered light signal 86 is a function of vertical position on the spectrograph slit 88, y, and wavelength λ once the light is dispersed by the spectrograph 69. The detected signal at pixel (m, n) can be related to the scattered light 80 and reference input beam 56 (Eₛ, Eᵣ) as:

I(λₘ,yₙ)= [symbol] Eᵣ(λₘ,yₙ)|² [symbol] + [symbol] Eₛ(λₘ,yₙ)|² [symbol] +2Re [symbol] Eₛ(λₘ,yₙ)E*ᵣ(λₘ,yₙ) [symbol] cos φ (1)

where φ is the phase difference between the two beams 70, 56 and [symbol] · [symbol] denotes an ensemble average in time. The interference term is extracted by measuring the intensity of the signal 70 and reference beams 56 independently and subtracting them from the total intensity.

In order to obtain depth-resolved information, the wavelength spectrum at each scattering angle is interpolated into a wavenumber (k=2π/λ) spectrum and Fourier transformed to give a spatial cross correlation, Γ_{SR}(z) for each vertical pixel yₙ:

Ε_{SR}(z,yₙ)=∫dke^{ikz} [symbol] Eₛ(k,yₙ)E*ᵣ(k,yₙ) [symbol] cos φ, (2)

The reference beam 54 takes the form:

Eᵣ(k)=Eₒexp[−((k−kₒ)/Δk)²]exp[−((y−yₒ)/Δy)²]exp[ikΔl] (3)

where kₒ (yₒ and Δk (Δy) represent the center and width of the Gaussian wave vector (spatial) distribution and Δl is the selected path length difference. The scattered light 80 takes the form

Eₛ(k,θ)=ΣⱼEₒexp[−((k−kₒ)/Δk)²]exp[iklⱼ]Sⱼ(k,θ) (4)

where Sⱼ represents the amplitude distribution of the scattering originating from the jth interface, located at depth lⱼ. The angular distribution of the scattered light 80 is converted into a position distribution in the Fourier image plane of lens L4 through the relationship y=f₄θ. For the pixel size of the CCD 90 (e.g., 20 μm), this yields an angular resolution (e.g., 0.57 mrad) and an expected angular range (e.g., 228 mrad).

Inserting Equations (3) and (4) into Equation (2) and noting the uniformity of the reference field 54 (Δy>>slit height) yields the spatial cross correlation at the nth vertical position on the imaging spectrograph 69:

$${Γ}_{SR} ( z , {y}_{n} ) = {\sum }_{j} \int d k {\vert {E}_{o} \vert}^{2} exp [ - 2 {( ( k - {k}_{o} ) / Δ k )}^{2} ] exp [ i k ( z - Δ l + {l}_{j} ) ] \times {S}_{j} ( k , {θ}_{n} = {y}_{n} / {f}_{4} ) cos φ . \qquad ( 5 )$$

Evaluating this equation for a single interface yields:

∇_{SR}(z,yₙ)=|Eₒ|²exp[−((z−Δl+lⱼ)Δk)²/8]Sⱼ(kₒ,θₙ=yₙ/f₄)cos φ. (6)

Here, it is assumed that the scattering amplitude S does not vary appreciably over the bandwidth of the source light 52. This expression shows that we obtain a depth resolved profile of the scattering distribution 80 is obtained with each vertical pixel corresponding to a scattering angle.

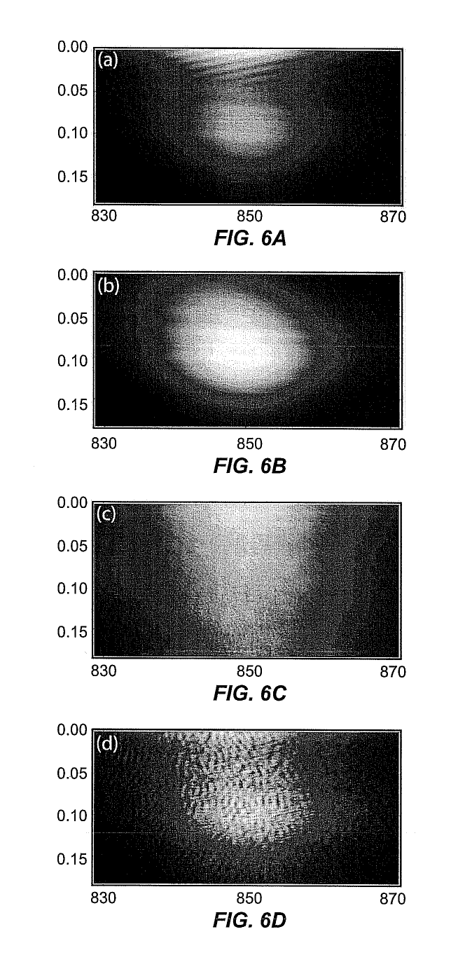

FIG. 6A shows typical data representing the total detected intensity (Equation (1), above) of the sum of the input beam 56 and the scattered light 80 by a sample of polystyrene beads, in the frequency domain given as a function of wavelength and angle, given with respect to the backwards scattering direction. In an exemplary embodiment, this data was acquired in 40 milliseconds and records data over 186 mrad, approximately 85% of the expected range, with some loss of signal at higher angles.

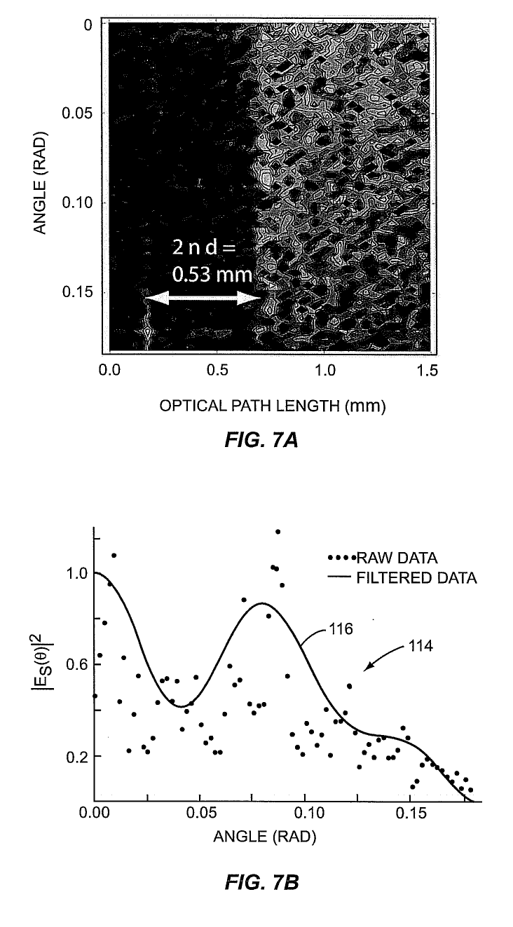

FIGS. 6B and 6C illustrate the intensity of the reference and signal fields 54, 70 respectively. Upon subtraction of the signal and reference fields 54, 70 from the total detected intensity, the mixed scattered light or interference data 86 between the two fields is realized as illustrated in FIG. 6D. At each angle, interference data 86 are interpolated into k-space and Fourier transformed to give the angular depth resolved profiles of the sample 58 as illustrated in FIG. 7A. The Fourier transform of the angle-resolved, cross correlated signal 86, which is the result of signal 80 scattered at a multitude of reflected angles off the sample 58 and obtained in the Fourier plane of lens L4 (78), produces depth-resolved information about the sample 58 as a function of angle and depth. This provides depth-resolved information about the sample 58. Because the angle-resolved, cross-correlated signal 86 is spectrally dispersed, the data acquisition permits data to be obtained in seconds or minutes. Information about all depths of the sample 58 at each of the multitude of different points (i.e., angles) on the sample 58 can be obtained with one scan on the order of approximately 40 milliseconds. Time domain-based scanning is required to obtain information about all depths of a sample at a multitude of different points, thus requiring more time and movement of the reference arm with respect to the sample. Time-domain based angle-resolved LCI (a/LCI) systems can still be provided that have the capability of examining and monitor tissue during the course of the same or concomitant medical procedure to determine if a therapeutic should be applied to the tissue. Examples of time-domain a/LCI scanning systems that can be employed in this regard will be described later below in this application.

In the experiments that produced the depth-resolved profile of the sample 58 illustrated in FIG. 7A, the sample 58 consists of polystyrene microspheres (e.g., n=1.59, 10.1 μm mean diameter, 8.9% variance, NIST certified, Duke Scientific) suspended in a mixture of 80% water and 20% glycerol (n=1.36) to provide neutral buoyancy. The solution was prepared to obtain a scattering length l=200 μm. The sample is contained in a round well (8 mm diameter, 1 mm deep) behind a glass coverslip (thickness, d˜170 μm) (not shown). The sample beam 70 is incident on the sample 58 through the coverslip. The round trip thickness through the coverslip (2nd=2(1.5) (170 μm)=0.53 mm—see FIG. 7A) shows the depth-resolved capability of the approach. The data is ensemble averaged by integrating over one mean free path (MFP). The spatial average can enable a reduction of speckle when using low-coherence light to probe a scattering sample. To simplify the fitting process, the scattering distribution is low pass filtered to produce a smoother curve, with the cutoff frequency chosen to suppress spatial correlations on length scales above 16 μm.

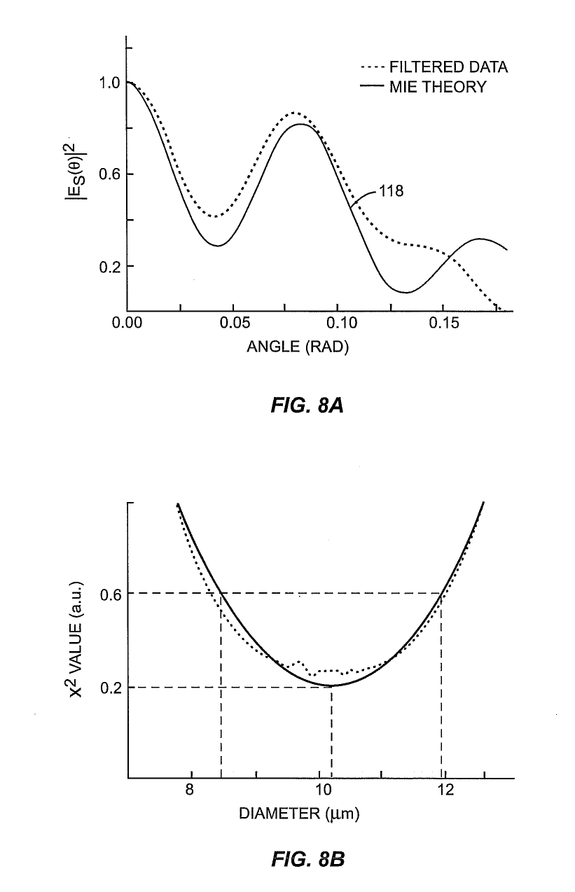

In addition to obtaining depth-resolved information about the sample 58, the scattering distribution data (i.e., a/LCI data) obtained from the sample 58 using the disclosed data acquisition scheme can also be used to make a size determination of the nucleus using the Mie theory. A scattering distribution 114 of the sample 58 is illustrated in FIG. 7B as a contour plot. The raw scattered data 112 about the sample 58 is shown as a function of the signal field and angle. A filtered curve is determined using the scattered data 114. Comparison of the filtered scattering distribution curve 116 (i.e., a representation of the scattered data 114) to the prediction of Mie theory (curve 118 in FIG. 8A) enables a size determination to be made.

In order to fit the scattered data 114 to Mie theory, the a/LCI signals are processed to extract the oscillatory component which is characteristic of the nucleus size. The smoothed a/LCI data 114 is fit to a low-order polynomial (4^{th} order was used for example herein, but later studies use a lower 2^{nd} order), which is then subtracted from the distribution 116 to remove the background trend. The resulting oscillatory component is then compared to a database of theoretical predictions obtained using Mie theory 118 from which the slowly varying features were similarly removed for analysis.

A direct comparison between the filtered a/LCI data 116 and Mie theory data 118 may not possible, as the chi-squared fitting algorithm tends to match the background slope rather than the characteristic oscillations. The calculated theoretical predictions include a Gaussian distribution of sizes characterized by a mean diameter (d) and standard deviation (6D) as well as a distribution of wavelengths, to accurately model the broad bandwidth source.

The best fit (FIG. 8A) is determined by minimizing the Chi-squared between the scattered data 116 and Mie theory (FIG. 8B), yielding a size of 10.2+/−1.7 μm, in excellent agreement with the true size. The measurement error is larger than the variance of the bead size, most likely due to the limited range of angles recorded in the measurement.

As an alternative to processing the a/LCI data and comparing to Mie theory, there are several other approaches which could yield diagnostic information. These include analyzing the angular data using a Fourier transform to identify periodic oscillations characteristic of cell nuclei. The periodic oscillations can be correlated with nuclear size and thus will possess diagnostic value. Another approach to analyzing a/LCI data is to compare the data to a database of angular scattering distributions generated with finite element method (FEM) or T-Matrix calculations. Such calculations may offer superior analysis as they are not subject to the same limitations as Mie theory. For example, FEM or T-Matrix calculations can model non-spherical scatterers and scatterers with inclusions while Mie theory can only model homogenous spheres.

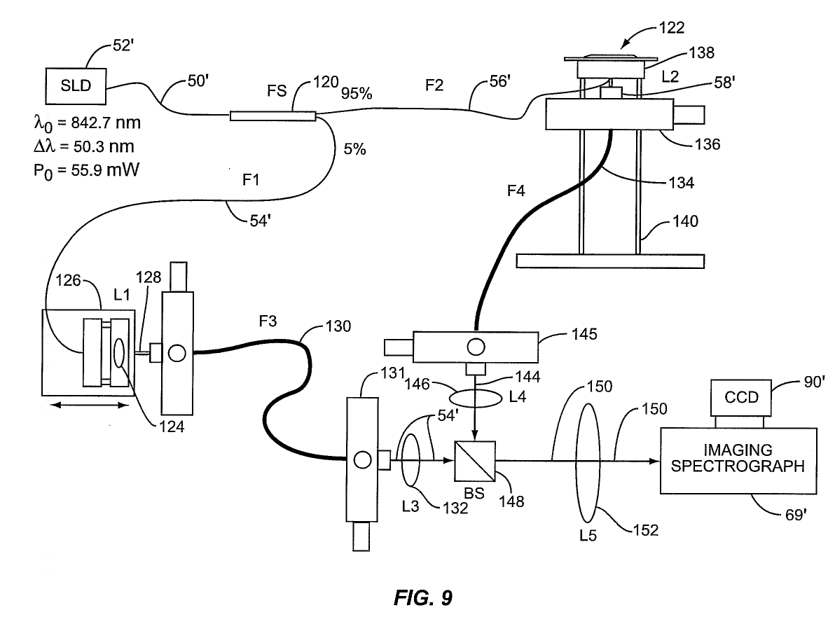

As an alternative embodiment, the present invention can also employ optical fibers to deliver and collect light from the sample of interest to use in the a/LCI system for endoscopic applications, such as that illustrated in FIG. 3 and those illustrated later in this application. This alternative embodiment is illustrated in FIG. 9.

The fiber optic a/LCI scheme for this alternative embodiment makes use of the Fourier transform properties of a lens. This property states that when an object is placed in the front focal plane of a lens, the image at the conjugate image plane is the Fourier transform of that object. The Fourier transform of a spatial distribution (object or image) is given by the distribution of spatial frequencies, which is the representation of the image's information content in terms of cycles per mm. In an optical image of elastically scattered light, the wavelength retains its fixed, original value and the spatial frequency representation is simply a scaled version of the angular distribution of scattered light.

In the fiber optic a/LCI scheme, the angular distribution is captured by locating the distal end of the fiber bundle in a conjugate Fourier transform plane of the sample using a collecting lens. This angular distribution is then conveyed to the distal end of the fiber bundle where it is imaged using a 4f system onto the entrance slit of an imaging spectrograph. A beamsplitter is used to overlap the scattered field with a reference field prior to entering the slit so that low coherence interferometry can also be used to obtain depth-resolved measurements.

Turning now to FIG. 9, the fiber optic faLCI scheme is shown. Broadband light 50′ from a broadband light source 52′ is split into a reference field 54′ and a signal input field 56′ using a fiber splitter (FS) 120. A splitter ratio of 20:1 is chosen in one embodiment to direct more power to a sample 58′ via a signal arm 122 as the light returned by the tissue is typically only a small fraction of the incident power.

Light in the reference field 54′ emerges from fiber F1 and is collimated by lens L1 1(124) which is mounted on a translation stage 126 to allow gross alignment of the reference arm path length. This path length is not scanned during operation but may be varied during alignment. A collimated beam 128 is arranged to be equal in dimension to the end 131 of fiber bundle F3 (130) so that the collimated beam 128 illuminates all fibers in F3 (130) with equal intensity. The reference field 54′ emerging from the distal tip of F3 (130) is collimated with lens L3 (132) in order to overlap with the scattered field conveyed by fiber F4 (134). In an alternative embodiment, light 54′ emerging from fiber F1 is collimated then expanded using a lens system to produce a broad beam.

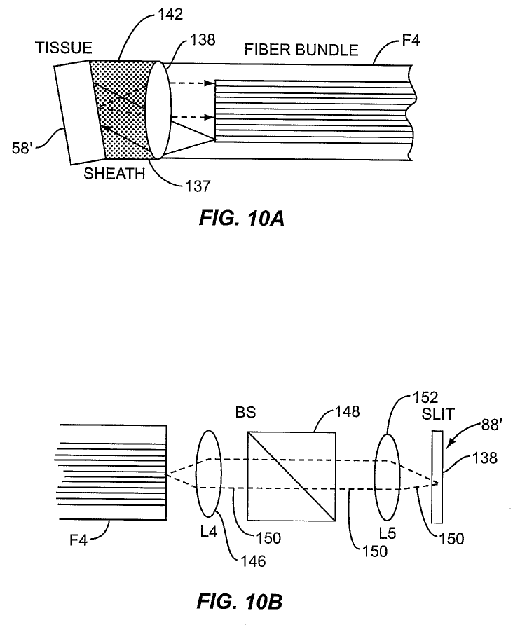

The scattered field is detected using a coherent fiber bundle. The scattered field is generated using light in the signal arm 122, which is directed toward the sample 58′ of interest using lens L2 (138). As with the free space system, lens L2 (138) is displaced laterally from the center of single-mode fiber F2 such that a collimated beam is produced which is traveling at an angle relative to the optical axis. The fact that the incident beam strikes the sample 58′ at an oblique angle is essential in separating the elastic scattering information from specular reflections. The light scattered by the sample 58′ is collected by a fiber bundle consisting of an array of coherent single mode or multi-mode fibers. The distal tip of the fiber is maintained one focal length away from lens L2 (138) to image the angular distribution of scattered light. In the embodiment shown in FIG. 10, the sample 58′ is located in the front focal plane of lens L2 (138) using a mechanical mount 136. In the endoscope-compatible probe shown in FIG. 9, the sample is located in the front focal plane of lens L2 (138) using a transparent sheath 142 (FIG. 10A).

As illustrated in FIG. 9 and also FIG. 10B, scattered light 144 emerging from a proximal end 145 of the fiber probe F4 (134) is recollimated by lens L4 (146) and overlapped with the reference field 54′ using beamsplitter BS (148). The two combined fields 150 are re-imaged onto the spectrograph slit 88′ of the imaging spectrograph 69′ using lens L5 (152). The focal length of lens L5 (152) may be varied to optimally fill the spectrograph slit 88′. The resulting optical signal contains information on each scattering angle across the vertical dimension of the slit 88′ as described above for the apparatus of FIGS. 4A and 4B.

It is expected that the above-described a/LCI fiber-optic probe will collect the angular distribution over a 0.45 radian range (approx. 30 degrees) and will acquire the complete depth resolved scattering distribution 114 in a fraction of a second.

There are several possible schemes for creating the fiber probe which are the same from an optical engineering point of view. One possible implementation would be a linear array of single mode fibers in both the signal and reference arms. Alternatively, the reference arm 136 could be composed of an individual single mode fiber with the signal arm 122 consisting of either a coherent fiber bundle or linear fiber array.

The fiber probe tip can also have several implementations which are substantially equivalent. These would include the use of a drum or ball lens in place of lens L2 (138). A side-viewing probe could be created using a combination of a lens and a mirror or prism or through the use of a convex mirror to replace the lens-mirror combination. Finally, the entire probe can be made to rotate radially in order to provide a circumferential scan of the probed area.

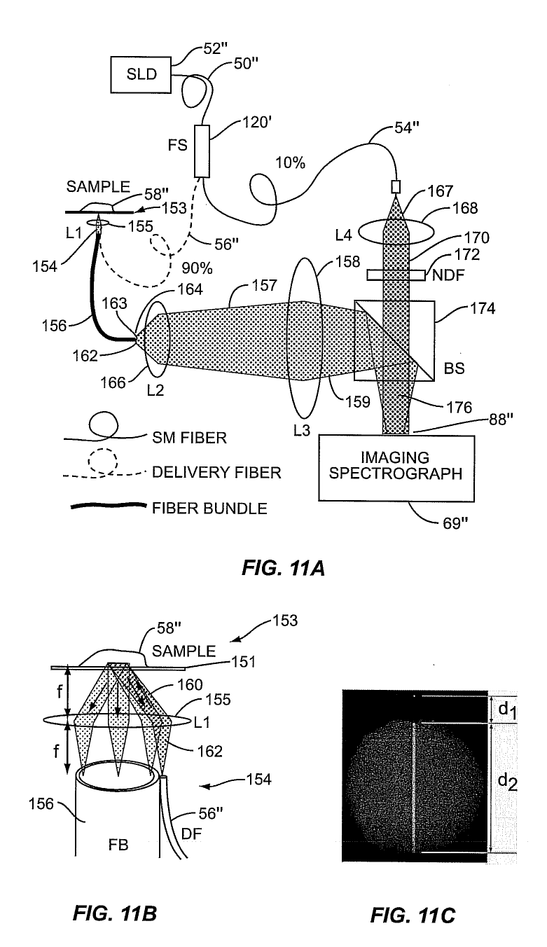

Yet another data acquisition embodiment of the present invention could be a faLCI system is based on a modified Mach-Zehnder interferometer as illustrated in FIG. 11A. The broadband light 50″ from a fiber-coupled superluminescent diode (SLD) source 52″ (e.g., Superlum, Pₒ=15 mW, λo=841.5 nm, Δλ=49.5 nm, coherence length=6.3 μm) is split into sample arm delivery fiber 56″ and a reference arm delivery fiber 54″ by a 90/10 fiber splitter FS (120′) (e.g., manufactured by AC Photonics). The sample arm delivery fiber 56″ can consist of either of the following for example: (1) a single mode fiber with polarization control integrated at the tip; or (2) a polarization maintaining fiber. A sample probe 153 is assembled by affixing the delivery fiber 56″ (NA≅0.12) along a ferrule 154 at the distal end of a fiber bundle 156 such that the end face of the delivery fiber 56″ is parallel to and flush with the face of the fiber bundle 156. Ball lens L1 (155) (e.g., f₁=2.2 mm) is positioned one focal length from the face of the probe 153 and centered on the fiber bundle 156, offsetting the delivery fiber 56″ from the optical axis of lens L1 (155). This configuration, which is also depicted in FIG. 11B, produces a collimated beam 160 (e.g., P=9 mW) with a diameter (e.g., 2f₁NA) of 0.5 mm incident on the sample 58″ at an angle of 0.25 radians, for example.

Scattered light 162 from the sample is collected by lens L1 (155) and, via the Fourier transform property of the lens L1 (155, the angular distribution of the scattered field 162 is converted into a spatial distribution at the distal face of the multimode coherent fiber bundle 156 (e.g., Schott North America, Inc., length=840 mm, pixel size=8.2 μm, pixel count=13.5K) which is located at the Fourier image plane of lens L1 (155). The relationship between vertical position on the fiber bundle, y′, and scattering angle, θ is given by y′=f₁θ. As an illustration, the optical path of light scattered 162 at three selected scattering angles is shown in FIG. 11B. Overall, the angular distribution is sampled by approximately 170 individual fibers for example, across a vertical strip of the fiber bundle 156″, as depicted by the highlighted area in FIG. 11C. The 0.2 mm, for example, thick ferrule (d₁) separating the delivery fiber 56″ and fiber bundle 156 limits the minimum theoretical collection angle (θ_{min,th}=d₁/f₁) to 0.09 radians in this example. The maximum theoretical collection angle is determined by d₁ and d₂, the diameter of the fiber bundle, by θ_{max,th}=(d₁+d₂)/f₁ to be 0.50 radians. Experiments using a standard scattering sample 162 indicate the usable angular range to be θₘᵢₙ=0.12 radians to θₘₐₓ=0.45 radians d₁, for example, can be minimized by fabricating a channel in a distal ferrule 163 (FIG. 11A) and positioning the delivery fiber 56″ in the channel. The fiber bundle 156 is spatially coherent, resulting in a reproduction of the collected angular scattering distribution at the proximal face. Additionally, as all fibers in the fiber bundle 156 are path length matched to within the coherence length, the optical path length traveled by scattered light 162 at each angle is identical. The system disclosed in “Fiber-optic-bundle-based optical coherence tomography,” by T. Q. Xie, D. Mukai, S. G. Guo, M. Brenner, and Z. P. Chen in Optics Letters 30(14), 1803-1805 (2005) (hereinafter “Xie”), incorporated by reference herein in its entirety, discloses a multimode coherent fiber bundle into a time-domain optical coherence tomography system and demonstrates that the modes of light coupled into an individual fiber will travel different path lengths. In the example herein of the present invention, it was experimentally determined that the higher order modes are offset from the fundamental mode by 3.75 mm, well beyond the depth (˜100 μm) required for gathering clinically relevant data. Additionally, the power in the higher order modes had a minimal affect on dynamic range as the sample arm power is significantly less than the reference arm power. Finally, it should be noted that while the system disclosed in Xie collected data serially through individual fibers, the example of the present invention herein uses 170 fibers to simultaneously collect scattered light across a range of angles in parallel, resulting in rapid data collection.

The angular distribution exiting a proximal end 164 of the fiber bundle 156 is relayed by the 4f imaging system of L2 (138) and L3 (132) (f₂=3.0 cm, f₃=20.0 cm) to the input slit 88″ of the imaging spectrograph 69″ (e.g., Acton Research, InSpectrum 150). The theoretical magnification of the 4f imaging system is (f₃/f₂) 6.67 in this example. Experimentally, the magnification was measured to be M=7.0 in this example with the discrepancy most likely due to the position of the proximal end 164 of the fiber bundle 156 with relation to lens L2 (166). The resulting relationship between vertical position on the spectrograph slit 88″, y, and θ is y=Mf₁(θ−θₘᵢₙ). The optical path length of the reference arm is matched to that of the fundamental mode of the sample arm. Light 167 exiting the reference fiber 54″ is collimated by lens L4 (168) (e.g., f=3.5 cm, spot size=8.4 mm) to match the phase front curvature of the sample light and to produce even illumination across the slit 88″ of the imaging spectrograph 69″. A reference field 170 may be attenuated by a neutral density filter 172 and mixed with the angular scattering distribution at beamsplitter BS (174). Mixed fields 176 are dispersed with a high resolution grating (e.g., 1200 lines/mm) and detected using an integrated, cooled CCD (not shown) (e.g., 1024×252, 24 μm×24 μm pixels, 0.1 nm resolution) covering a spectral range of 99 nm centered at 840 nm, for example.

The mixed fields 176, a function of wavelength, λ, and θ, can be related to the signal and reference fields (Es, Er) as:

I(λₘ,θₙ)= [symbol] Eᵣ(λₘ,θₙ)|² [symbol] + [symbol] Eₛ(λₘ,θₙ)|² [symbol] +2Re [symbol] Eₛ(λₘ,θₙ)E*ᵣ(λₘ,θₙ)cos(φ) [symbol] , (7)

where φ is the phase difference between the two fields, (m,n) denotes a pixel on the CCD, and [symbol] . . . [symbol] denotes a temporal average. I(λₘ,θₙ) is uploaded to a personal computer (PC) using LabVIEW software manufactured by National Instruments and processed in 320 ms to produce a depth and angle-resolved contour plot of scattered intensity. The processing of the angle-resolved scattered field to obtain depth and size information described above, and in particular reference to the data acquisition apparatus of FIGS. 4A and 4B, can then used to obtain angle-resolved, depth-resolved information about the sample 58″ using the scattered mixed fields 176 generated by the apparatus in FIG. 11A.

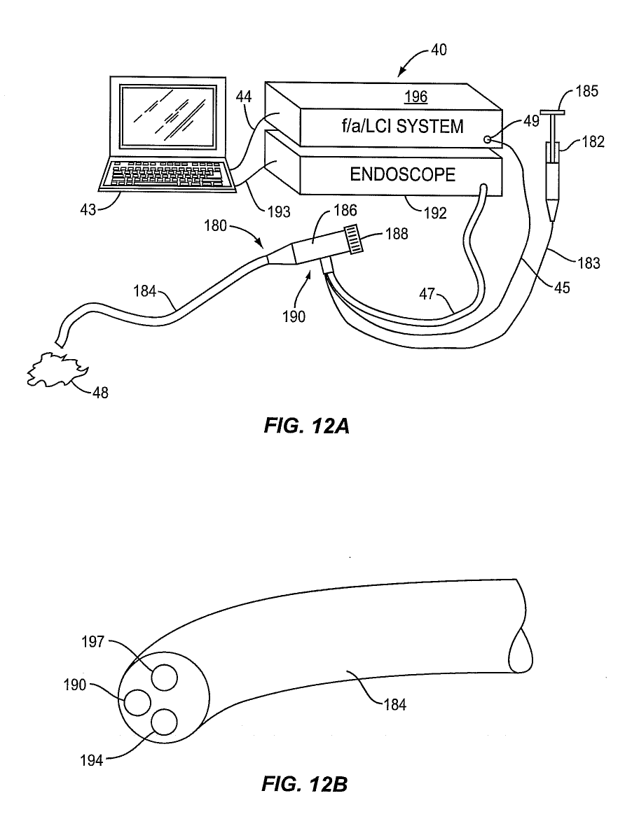

This disclosure expands the capability of one or more therapeutics to the system. The system may or may not be used to collect biopsy samples. FIGS. 12A and 12B provides a general example of a real-time f/a/LCI system 40, which may be the faLCI system previously described above. The faLCI system 40 is integrated with a multi-channel endoscopic probe 180 with an integrated therapeutic, which in this example is a liquid that is controlled by a manual syringe 182. In this manner, a therapeutic can easily be delivered to the same tissue that is analyzed using the real-time f/a/LCI system 140 while the endoscope is used by a physician to monitor the actual tissue 58 being examined. In this regard, the endoscopic probe 180 consists of a flexible shaft 184 connected to a body 186 that contains an eyepiece 188 for viewing through the visual channel of the endoscopic probe 180. Integrated into the endoscopic probe 180 is a channel 190 for light, air, and water to pass down through a shaft 47 into the endoscopic probe 180 and for a visual image of the tissue 48 to pass back up to the eyepiece 188. As illustrated in FIG. 12B, the real-time f/a/LCI system 40 is integrated via a separate channel 194 and interfaces with the f/a/LCI control box 196 (FIG. 12A), which may or may not interface to a separate computer 43. A therapeutic that can be administered passes down yet another integrated channel 198 and is manually administered by the operator.

In this example, the endoscopic probe 180 interfaces with an endoscope control box 192, which is the source of anything passing into the endoscopic probe 180 and the receiver for visual information returning from the endoscopic probe 180. In many cases, the visual image of the tissue 48 is displayed on a screen allowing the operator to see inside the patient without using the eyepiece 188. In this regard, the endoscope control box 192 may be under the control of the computer 43 via a communications line(s) 193 to provide control and for receiving images of the patient's tissue if the endoscopic probe 180 employs a camera.

Note that the endoscopic probe 180, the real-time f/a/LCI system 40, and therapeutic functions are shown as independent connections and control boxes in FIGS. 12A and 12B, but this is for illustrative purposes only and is not a requirement. The computer 43 is shown as independent and connected to the real-time f/a/LCI system 40 and the endoscopic probe 180; this is also not a requirement. The computer 43 may be completely integrated or independent and may or may not be connected to portions of the system in lieu of the real-time f/a/LCI system 40. A computer 43 as used herein means any computing device. Note that this configuration of the real-time f/a/LCI system 40 in FIGS. 12A and 12B will work with numerous therapeutics. The first one described is a current experimental technique: radio frequency (RF) ablation. RF ablation consists of dosing the tissue with sufficient radio frequency energy to kill a layer of cells at the surface of the tissue without harming deeper tissue. This may vary for tissue type, but for esophageal tissue is from one (1) Joule/cm² to 50 Joule/cm² with a duration of less than one (1) second and preferably less than 0.25 seconds, as described, for example, in U.S. Patent Application Publication No. US2004/0215296, incorporated herein by reference in its entirety.

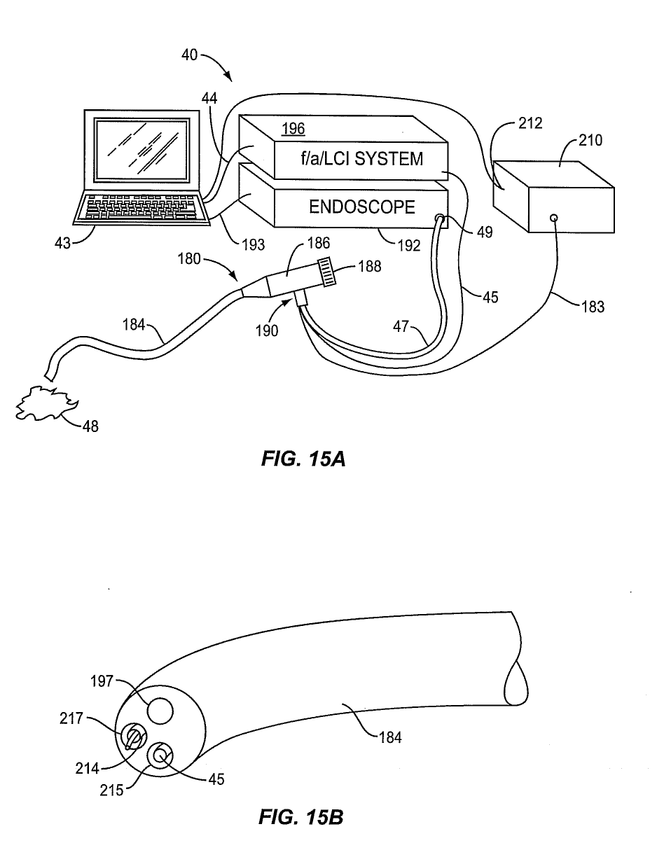

Another class of therapeutics is applied substances. The therapeutic substance could take the form of a liquid, gel, aerosol, or gas, as examples. This could include, but is not limited to, drugs, compounds, and/or elements that cause a chemical reaction at the tissue site and/or substances that affect the tissue in a physical manner such as hot or cold liquids or acids or bases. Collectively, these administered therapeutics will be referred to as “substances.” FIGS. 12A and 12B, previously described above, provided one exemplary implementation where the therapeutic substance is delivered via a tube 183 and the flow is controlled via a manual plunger 185 in the syringe 182. Another exemplary implementation is shown in FIGS. 15A and 15B, whereby an automatic dispenser 210 controls the substance flow and is in turn controlled either via input 212 on the dispenser 210, or via electronic control from the same computer 43 via communication line 213 that collects and analyzes the data from the real-time f/a/LCI system 40. As discussed in greater detail below, this control can be manual via the operator, fully automatic via the software on the computer, or somewhere in between. This system will enable localized controlled delivery of substances to tissue diagnosed as abnormal. Tissue extracted from the body and identified as pre-cancerous can be treated but there is no way to verify that the same effect will occur in vivo. The ideal scenario would be the ability to scan the tissue, dose the tissue with an experimental compound and then re-scan the tissue on a periodic basis to observe the effect of the compound over time. The system in general and this implementation specifically will offer this capability.

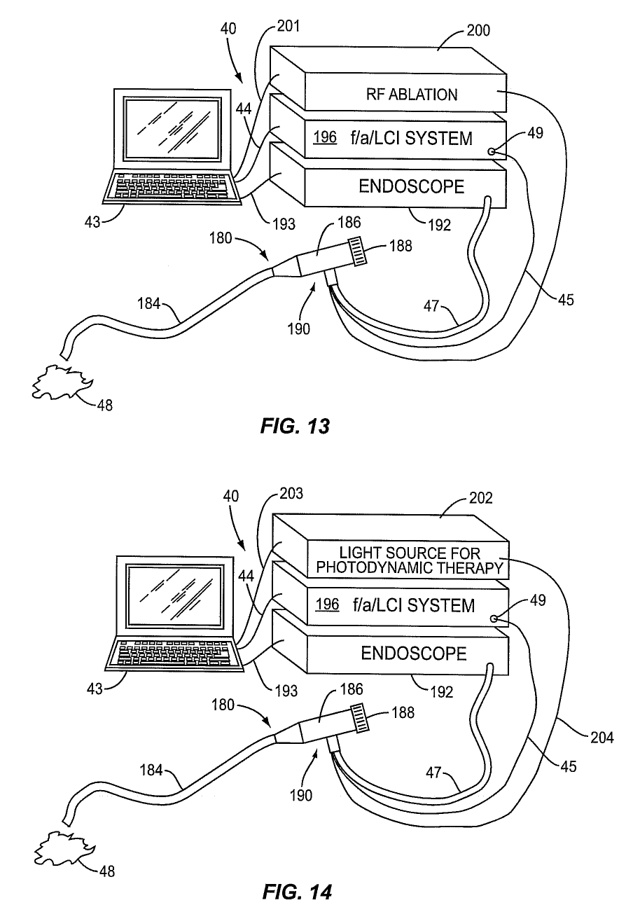

FIG. 13 illustrates another implementation, consisting of the endoscope 192, the real-time f/a/LCI system 40, and an RF ablation system 200 as the therapeutic system of choice. These systems are shown as fully integrated into the endoscope control box 192 with independent control boxes with full system control managed view to a user interface on the computer 43. One possible method of operation is as the operator scans the tissue using the real-time f/a/LCI system 40 and the endoscopic probe 180, anytime abnormal tissue is detected, the operator triggers the RF ablation system 200 to deliver a dose of RF energy to the tissue 48. The RF ablation system 200 may be under the control of the computer 43 via a communication line(s) 201. Examples of RF ablation systems are disclosed in U.S. Pat. Nos. 6,551,310 and 6,551,310, and in U.S. Published Patent Application No. US2004/0215296A1, each of which is incorporated herein by reference in its entirety.

Another therapeutic that can be used is photodynamic therapy. Here, the patient is given a drug called a photosensitizer and then exposed to a particular type (wavelength) of light, for example, the light from a Nd:YAG laser at a wavelength of 630 micrometers. Numerous photosensitizers are known in the art, including but not limited to porfimer sodium, chlorins, bacteriochlorins, purpurins, benzoporphyrins, texaphyrins, etiopurpurins, naphthalocyanines and phthalocyanines. The drug interacts with the light and produces a form of oxygen that kills nearby cells. The photosensitizer is typically injected into the blood, and between 24 and 72 hours later, the tumor is exposed to light. This time window is set by the fact that the photosensitizer remains in the cancer cells longer than in other cells in the body. The photodynamic therapy has several side affects including damage to tissue near the tumor and sensitizing the skin and eyes to light for up to six weeks after the treatment. A photodynamic therapy system can be integrated with the real-time f/a/LCI system 40 and the endoscopic probe 180. One possible implementation of an integrated photodynamic therapy system with a real-time f/a/LCI system is illustrated in FIG. 14.

As illustrated in FIG. 14, light from a photodynamic therapy system 202 is controlled through a shaft 204 into an instrument channel of the endoscopic probe 180, which may be an auxiliary instrument channel 198 like illustrated in FIG. 12B. Further examples of photodynamic therapy and photosensitizers are disclosed in U.S. Pat. Nos. 5,330,741; 5,506,255; and 5,591,847 which are incorporated herein by reference in its entirety.

The photodynamic therapy system 202 may be controlled by the computer 43 via a communications line(s) 203. The real-time f/a/LCI system 40 can provide guidance information that will help pinpoint where to use the photodynamic therapy on tissue 48. An advantage of guiding the photodynamic therapy should be reduced damage to nearby, non-cancerous tissue. Care would need to be taken to ensure that the light used for the real-time f/a/LCI system 40 does not activate the photodynamic therapy system 202 in a harmful manner. Some possible solutions include using low enough power levels for the real-time f/a/LCI system 40 as not to activate the photodynamic therapy system 202 to a harmful level or use a wavelength for the real-time f/a/LCI system 40 that is out of the range of the activation wavelength(s) for the photodynamic therapy system 202.

The endoscopic probe 180 may employ single or multi-instrument channels. A dual instrument channel variation is illustrated in FIG. 15B. As illustrated therein, the f/a/LCI probe 45 passes down one instrument channel 215 to access the tissue 48. A therapeutic substance can be administered by a therapeutic applicator or probe 214 via a second instrument channel 217 of the endoscopic probe 180. Operation is conceptually similar to the case where the probes 45, 214 are integrated, as provided in FIG. 15A. Variations include the case where the f/a/LCI probe 45 is integrated and the therapeutic probe 214 is administered via an instrument channel and vice versa. Another is the case where a single instrument channel endoscope is used, and the f/a/LCI probe 45 and the therapeutic probe 214 are administered sequentially via the single instrument channel. In other words, the f/a/LCI probe 45 is passed down an instrument channel, measurements or scanning occurs and when an area requiring treatment is detected, the f/a/LCI probe 45 is pulled out of the instrument channel, and the therapeutic probe 214 is passed down the instrument channel and delivered. Another variation is that more than one therapeutic can be used in a same or concomitant medical procedure.

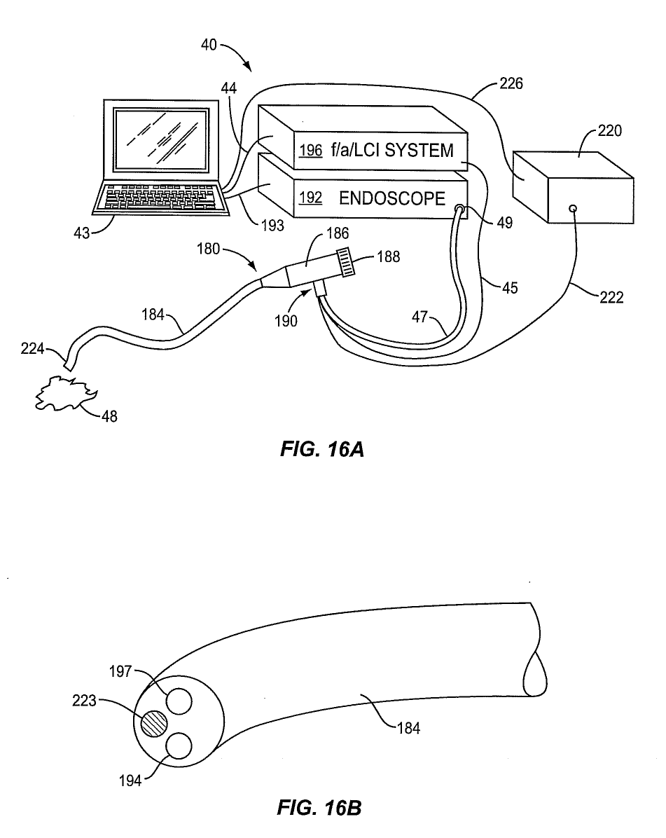

Another variation on this integrated system is the use of a hot or cold therapeutic to ablate or kill the abnormal tissue. The tissue can be locally heated or burned to destroy the cells. Alternately, the tissue could be chilled or frozen to achieve the same effect. There are numerous system implementations that will achieve this effect. A partial list includes placing a small heating coil at or near the end of the endoscopic probe 180 that is controlled by heater control unit 220 that in turn is controlled by the computer 43, as illustrated in FIG. 16A. The heater control unit 220 may also be integrated into the f/a/LCI control box 196. A conductor 222, such as a copper wire for example, is heated in the heater control unit 220 and conducts heat down the conductor 222 to the tissue 48 in the body. Using an instrument channel 223 in the endoscopic probe 180, heated or chilled air or liquid is passed down and administered to the tissue 48. Alternatively, using a thermoelectric cooler (TEC) that is either in the heater control unit 220 where the cold is conducted down to the tissue 48 via the conductor 222 or is physically located at or near the tip end 224 of endoscopic probe 180 and controlled via an electronic connection 226 to the heater control unit 220 can be used. In another embodiment, cryoablation may be used to treat the abnormal tissue. An example of a device to perform cryoablation is disclosed in U.S. Pat. No. 7,255,693, which is incorporated herein by reference in its entirety.

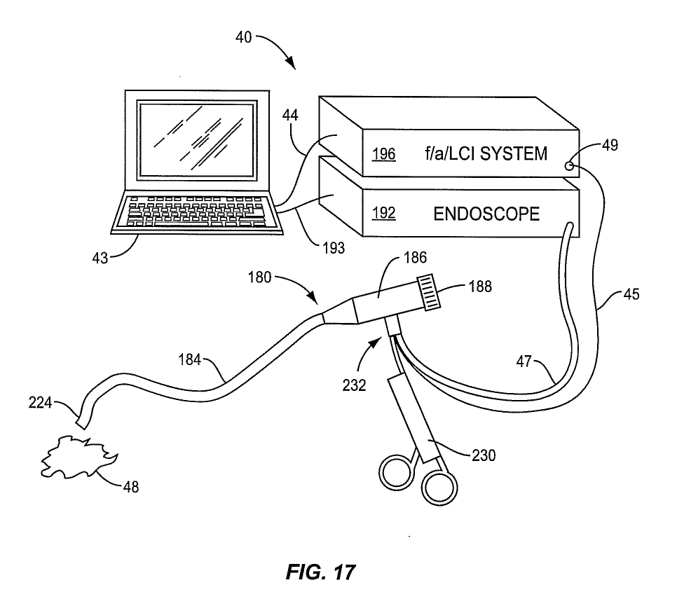

Another class of therapeutics involves removal of the non-normal (pre-cancerous or cancerous) tissue. This could be done via a variety of methods including cutting, scraping, using a punch biopsy, using an alligator clip biopsy and many others. One possible implementation is shown in FIG. 17 where a manual external control is used to surgically cut out tissue of concern. In this regard, a surgical instrument 230 can be provided and inserted into an instrument channel 232 of the endoscopic probe 180. The surgical instrument 230 allows removal of tissue 48 while the real-time f/a/LCI system 40 and the endoscope 192 are used to monitor and diagnose the tissue 48. There are multiple procedures that could be used to surgically remove tissue. One might be to scan the full area of concern, map out diseased tissue, go back and surgically remove tissue and then re-scan the area of concern to verify that the diseased tissue has been removed. Another might be to scrape out tissue as the real-time f/a/LCI system 40 scan occurs. Another would be to use standard biopsy tools to remove tissue that has been identified as of concern with a possible additional re-scan to verify that all tissue of concern has been removed.

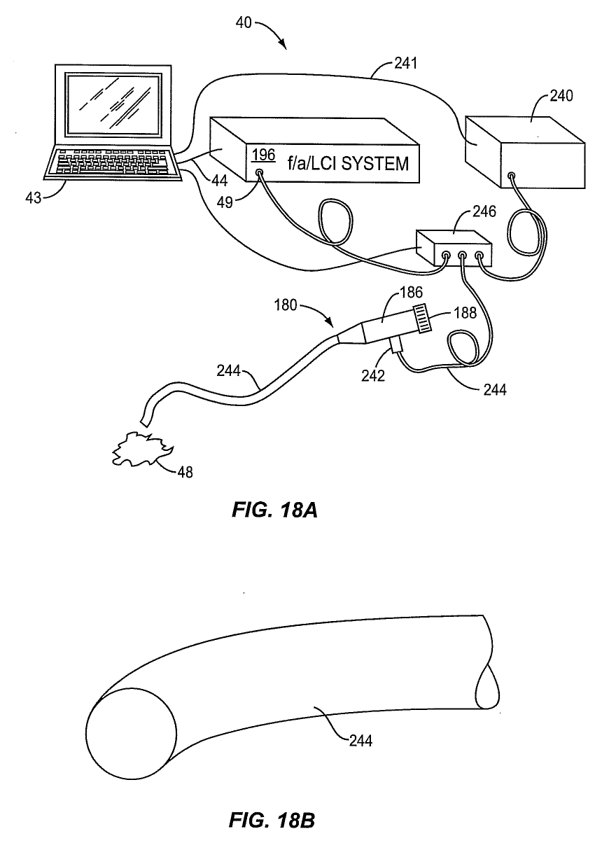

Another implementation is illustrated in FIGS. 18A and 18B and employs a single channel endoscopic probe 180 where the real-time f/a/LCI system 40 and a therapeutic system 240 are delivered via the same optical fiber or fiber bundle over a single channel 242. The therapeutic could be light ablation of the tissue 48 where the high power light travels down the same fiber or fiber bundle 45 used by the real-time f/a/LCI system 40 to diagnosis the tissue 48 since both light ablation and the real-time f/a/LCI system 40 employ light as their means of performance. A single fiber or fiber bundle 244 comes out of the endoscopic probe 180 on the patient side at the tissue 48. The single fiber or bundle 244 is then connected to an optical switching device 246 that connects the fiber 244 to either the real-time f/a/LCI system 40 or a high power source therapeutic system 240. The high power source therapeutic system 240 may be under control of the computer 43 via communication line 241. This optical switching device 246 may be controlled by the computer 43 in conjunction with the real-time f/a/LCI system 40 and the high power source therapeutic system 240. Typical operation might include scanning the tissue 48 with the single channel endoscopic probe 180 and triggering the high power source therapeutic system 240 to ablate the tissue 48 when an abnormal condition is detected. This embodiment may be useful for reaching tissue 48 that may not be accessible with the larger multi-channel endoscopes used, for example, in the esophagus or colon. Examples include endoscopes for reaching the bladder or the pancreas where access paths are a few millimeters or less in size. By employing the same fiber or fiber bundle 244 for the diagnosis and therapeutic will enable the operator to survey and treat tissue that might otherwise be inaccessible.

Note that the high power source therapeutic system 240 can either be continuous wave (CW) in operation or pulsed. Any wavelength can be used conceptually, selection will be driven by availability of sources and which wavelength(s) provide the best interaction with tissue to ablate abnormal tissue while minimizing effects on adjacent healthy tissue. Also, the multiple boxes shown for the computer 43, real-time f/a/LCI system 40, high power light source 240, and optical switching device 246 may be consolidated into fewer packages or devices.

The real-time f/a/LCI system 40 may also be used in conjunction with nanoparticles to modify the signal generated by the interaction with the sample and/or treat a condition within the sample. As an example, nanoparticles might be used to increase the optical contrast between the cell and the cell nuclei to increase the signal strength generated by the real-time f/a/LCI system 40. This may enable deeper penetration in the sample, which would be advantageous in many applications including the detection of skin cancer. Skin cancer is not normally detectable by f/a/LCI because the precancers or cancers start about one (1.0) millimeter below the surface and insufficient light reaches that depth and is scattered back. Increasing contrast may reduce the amount of light required to generate an f/a/LCI signal enabling deeper penetration in the tissue. Another application of f/a/LCI with nanoparticles is in the treatment of precancers or cancers. Nanoparticles can be used in a variety of treatment options for cancers, including using the nanoparticles which are toxic or carry toxic substances to kill precancerous or cancerous cells or tissue or using nanoparticles for photodynamic therapy where the nanoparticles absorb a light (perhaps from a specific wavelength or wavelength range) and heat up, thereby killing cells. For example, a real-time a/f/LCI system can be used to identify and diagnose the presence of pre-cancerous or cancerous tissue, and then during the same or concomitant medical procedure the physician can treat the tissue with the nanoparticles. Several such uses or therapies utilizing nanoparticles are known in the art as shown by the following references each of which is incorporated herein: O'Neal et al. Photo-thermal tumor ablation in mice using near infrared-absorbing nanoparticles, Cancer Letters 209:171-176 (2004); Gu et al., Targeted Nanoparticles for Cancer Therapy, NanoToday 2:14-21 (2007); Loo et al., Nanoshell-Enabled Photonics-Based Imaging and Therapy of Cancer, Technology in Cancer Research & Treatment, 3: 33-40 (2004).

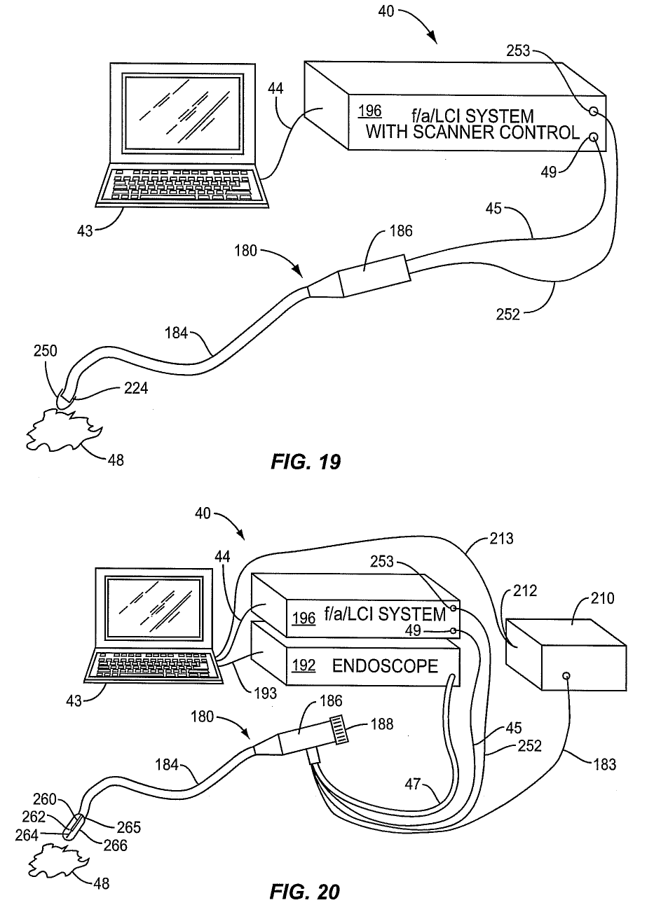

Another embodiment is to use a standalone real-time f/a/LCI system 40 to provide monitoring of an area of tissue 48 with a therapeutic provided separately. This is illustrated by example in FIG. 19. The common components in the system have been previously described and will not be repeated herein. After the real-time f/a/LCI system 40 is used to monitor the tissue, it is removed and then, either immediately or at a later time, a therapeutic can be administered to the tissue 48, if needed or desired, based at least in part on the information obtained from the real-time f/a/LCI system 40 monitoring. The real-time f/a/LCI system 40 could access the tissue via an endoscope of any of the forms previously described or may be a standalone real-time f/a/LCI system 40 capable of accessing tissue on its own. The therapeutic used might be any of the ones discussed in this disclosure or another therapeutic.

Another implementation of the real-time f/a/LCI system 40 can be used in conjunction with an endoscope and scanning mechanism that permits the real-time f/a/LCI system 40 to scan more than one spot on the tissue 48. FIG. 20 shows one possible implementation where a balloon 266 (or other device) is used to fix the location of the tissue 48 relative to an f/a/LCI scanner 262. In this example, the scanner 262 is fixed to a scanner head 265 and rotating mechanism 260 to be controlled to rotate in a spiral pattern to cover the tissue 48 section from bottom to top. This implementation may be faster than point by point coverage and may give a more uniform sampling of the tissue. An integrated therapeutic applicator 264 can be employed to deliver a therapeutic to the tissue 48, as previously discussed. There are also multiple options for use of this system in FIG. 20. One is the case where the tissue 48 is treated as the scan occurs. This may either be automatic or manual and may require a therapeutic that can pass through the balloon 266, such as light, heat, cold, etc. Another case is where a scan is taken of the tissue 48 and then the operator goes back and treats the tissue 48 based on data from the scan. There may be another scan to verify that the tissue is treated. Again, this may be manual or automatic.

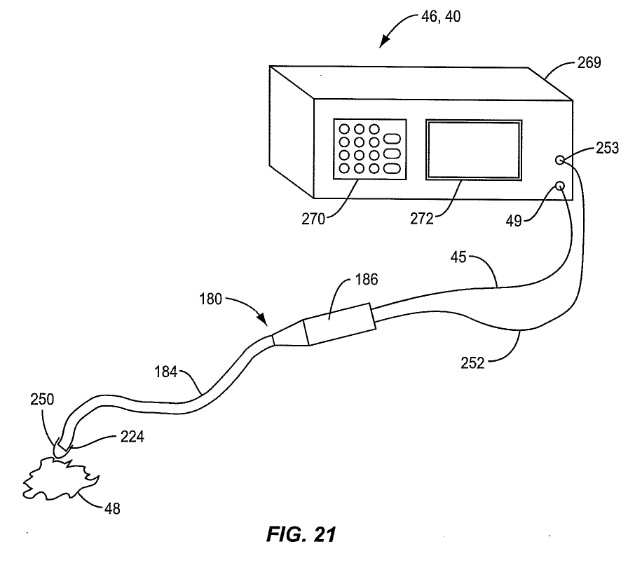

The exemplary systems illustrated thus far have shown an independent computer 43 as part of the system. This is not a requirement. However, the processing of the f/a/LCI information regarding the tissue could be done by any type of computer, such as a laptop, desktop, remote computer (including one connected by a wireless network), or other. There may be varying levels of physical integration. FIG. 21 shows a system with the computer 43 fully integrated into the real-time f/a/LCI system 40 in a chassis or box 269. This could be accomplished by a computer on a printed circuit board (PCB) board along with a liquid crystal display (LCD) display screen 272 and control panel 270, or some other configuration. The processing of the information regarding the tissue could also be performed in a separate system. The processing could be performed in one or more computers, one or more microprocessor, one or more digital signal processors (DSPs), one or more field programmable gate arrays (FPGAs), or some combination of these or other processing devices. Likewise, the external processing may occur in system with some combination of computers, microprocessors, DSPs, and/or FPGAs. It may also be the case that the external processing does not occur in the same location but may be in a different location and connected by the some communications system including, but not limited to, wireless, WiFi, Ethernet, serial or other. Also note that the communication between the chassis 269 and the external processing may occur via any number of communication methods including universal serial bus (USB), Firewire, Ethernet, WiFi, other serial (RS-232, etc) or other method.

There is a range of automation that can be achieved with this system and all levels are intended to be covered by the present invention. As examples, low automation might be the case where the real-time f/a/LCI system generates information and displays it to the screen. Using this information, the operator delivers some dosage of some therapeutic to the tissue. In this case, there may be no electronic connection between the computer and the endoscope or the therapeutic control. A middle level of automation might be the case where there is a connection between the computer and the therapeutic delivery system and the computer determines the dosage level based on information from the real-time f/a/LCI system and internal algorithms. The operator would control when the therapeutic is delivered, but the dosage is determined via software. A very high level of automation might be the case where the therapeutic is delivered independent of operator control. As the tissue is being scanning (either manually or automatically), the computer can control the delivery of the therapeutic based on information received from the real-time f/a/LCI system and internal algorithms.

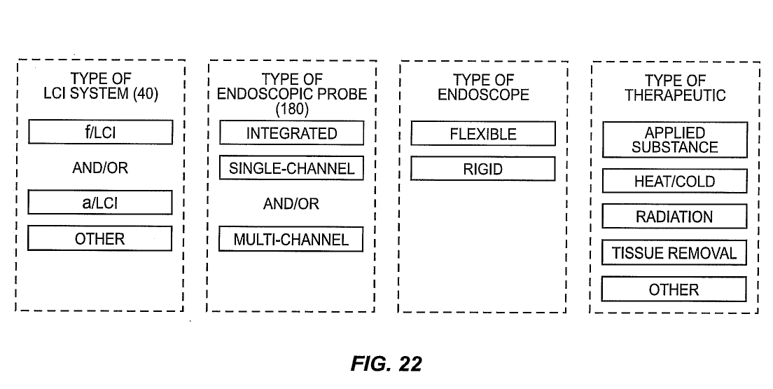

There are numerous possible configurations of the real-time f/a/LCI system 40 and therapeutic delivery techniques described above. FIG. 22 summarizes some of these possibilities. The real-time f/a/LCI system 40 may be a faLCI system, an aLCI system, or an fLCI system, some of which have been previously described and some of which will be described below in this application. The endoscopic probe 180 employed may be an integrated, single-channel, or multi-channel endoscope. The channels may be “integrated” in that they are physically part of the endoscope or the channels may be open passageways through the scope that any number of instruments or accessories may pass through. Typically the more channels an endoscope has, the larger the radial size, thus potentially limiting where the endoscope may go in the body. Endoscopes come in a variety of configurations; the endoscopy portion that goes into the patient may either be a rigid or flexible tube. Typically rigid tubes are limited to 20 to 30 centimeters in length, while flexible tubes may be several meters long. Finally, for this system, there are numerous types of possible therapeutics, which may influence the design of a particular version of the integrated system. The therapeutic can be an applied substance, heat/cold application, radiation, tissue removal, or other therapeutic.

Some of the therapies discussed have been localized or regional in nature. f/a/LCI offers an advantage here by pinpointing the location(s) to apply one or more of these therapies. f/a/LCI and the information generated by real-time f/a/LCI systems may also be used to guide or determine the use of other therapies which may involve the whole body or areas outside the location where the pre-cancer or cancer may be found. This included many of the therapies used today including radiation, stereotactic radiosurgery or therapy (which uses multiple radiation beams to irradiate small targets with minimum impact to adjacent tissue, also known as gamma knife), surgery (including, but not limited to general, Moh's surgery, laparoscopic or minimally invasive surgery (MIS) and robotically assisted MIS), and chemotherapies (including both oral and injected chemotherapies). The real-time f/a/LCI may be used as part of a procedure for gating the use of these therapies.

In addition, the f/a/LCI systems disclosed herein can be used to detect in tissue the margin or boundary between pre-cancerous, cancerous or diseased cells and normal cells. Repeated application of real-time f/a/LCI is then used to direct the serial surgical removal of all or nearly all the pre-cancerous or cancerous cells in the same or concomitant medical procedure. Such combination of real-time f/a/LCI optical biopsy and surgical removal of pre-cancerous, cancerous or diseased issue can be applied to any organ of tissue of the body using the methods, processes, techniques and systems of the present inventions. As another option, these therapies (in particular, the chemotherapies) may be used in conjunction with one or more of the localized treatment options. As an example, a location in the esophagus may be identified as pre-cancerous by an f/a/LCI system leading to an RF ablation treatment for that area of the esophagus and a course of chemotherapy.

Early detection by the f/a/LCI may enable not only the use of chemotherapies, but also chemopreventatives that have been developed or are under development. These chemopreventatives have not been widely deployed because there may be no good way to identify pre-cancerous conditions at an early enough stage, and because of the difficulty in identifying and testing potential chemopreventatives because of the issues in identifying pre-cancerous conditions at an early enough stage and conducting longitudinal testing to validate the effectiveness of these chemopreventatives. One possible example of this would be the identification of a pre-cancerous lesion in an esophagus where the patient then undergoes a course of injected (or oral) chemopreventative followed by f/a/LCI monitoring exams at one or more time points to verify the reduction or elimination of the pre-cancerous lesion.

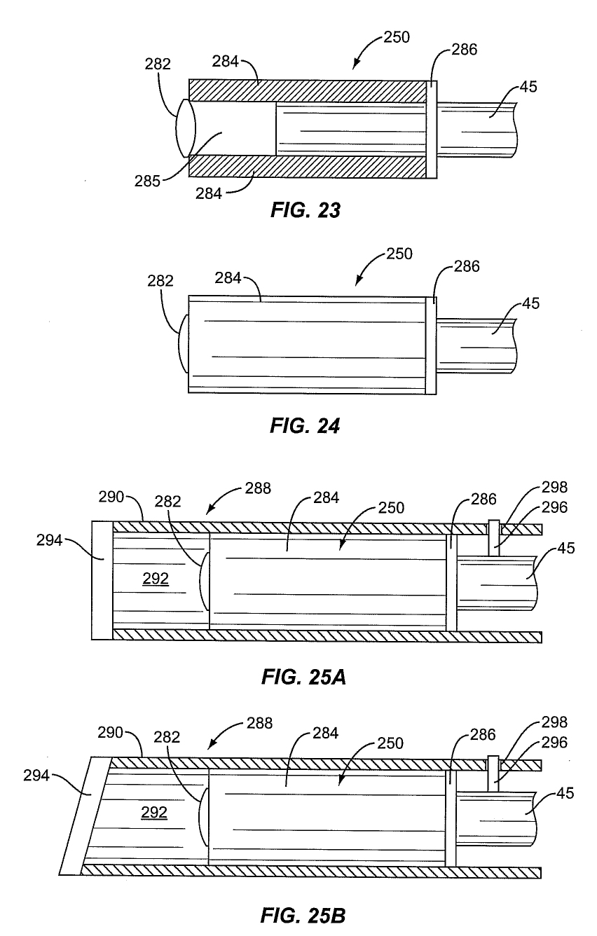

With the above backdrop, more detail regarding possible aspects of the systems are now described. In certain systems illustrated and previously described above, an endoscope probe tip 250 is shown in certain embodiments as a protective cover. The probe tip 250 may be disposable as a convenient means to keep the tip end 224 of the endoscope shaft 184 sterile so it can be used for multiple patients. In this regard, FIGS. 23-29 illustrate various examples of probe tips 250 that may be employed if the f/a/LCI probe 45 is employed in an instrument channel in the endoscope shaft 184. In general, the probe tip 250 can include a protective sheath over the optical fiber or bundle of the f/a/LCI probe 45. The probe tip 250 provides a sterile interface between the optical fiber probe 45 and the tissue surface 58 under examination during endoscopic applications. Because the probe tip 250 may be employed in optical spectroscopic techniques, the probe tip 250 includes an imaging element (e.g., lens) to capture reflected light from the tissue 48. The probe tip 250 is adapted to maintain the positioning of the imaging element relative to the optical fiber to properly pass reflected light from the tissue 48 to the optical fiber within the f/a/LCI probe 45.

As illustrated in FIG. 23, a probe tip 250 is provided in accordance with one embodiment. The probe tip 250 may be employed in any embodiment previously described, but may be particularly useful for a combined f/a/LCI probe 45 and therapeutic probe 214. FIG. 24 illustrates the probe tip 250, but in solid view. The probe tip 250 is adapted to cover the distal end of an optical fiber probe 45 used in an endoscopic imaging system, including those described above. If applied, the distal ends of the delivery fiber and fiber bundle 48 will be contained within the probe tip 250, as illustrated in FIG. 23.

One function of the probe tip 250 can be to create a fixed geometry between an optical fiber probe 45, an imaging element, and the tissue 48 under examination. Thus, a first component that can comprise the probe tip 250 is a means to locate an imaging element, such as a lens 282, relative to the fiber optic or bundle probe 45. FIG. 23 shows a cutaway schematic of the use of a fixed sheath 284 comprised of a cylindrically-shaped outer wall having a hollow portion 285 placed over and surrounding the distal end of the fiber probe 45 to position the lens 282. In this embodiment, the fixed sheath 284, having a fixed length, is placed over the fiber bundle 45 with a retaining ring 286 used to maintain the fixed distance between the fiber bundle 45 and the lens 282. The fixed sheath 284, by being fixed, possesses a rigid construction to maintain the required positioning of the lens 282 relative to the fiber probe 45. The lens 282 is located on a distal end of the fixed sheath 284. The fixed sheath 284 can be affixed to the fiber probe 45 with an adhesive, or can be attached to the retaining ring 286 using a flange or other locking mechanism. This configuration can be modified to include other types of optical elements or multiple optical elements (lenses, etc.).

If the probe tip 250 is employed in a real-time f/a/LCI system 40, the lens 282 can be placed approximately one focal length away from the fiber probe 45. This may be required for the lens 282 to properly capture the reflected angular distribution of light from the tissue for analysis. In alternate embodiments, the lens 282 can be positioned such that an individual single or multimode fiber or an array of such fibers is maintained at the focus of the lens 282. In other embodiments, the imaging lens 282 can be positioned at other distances from the fiber optic probe 45, which are different than the focal length of the lens 282.

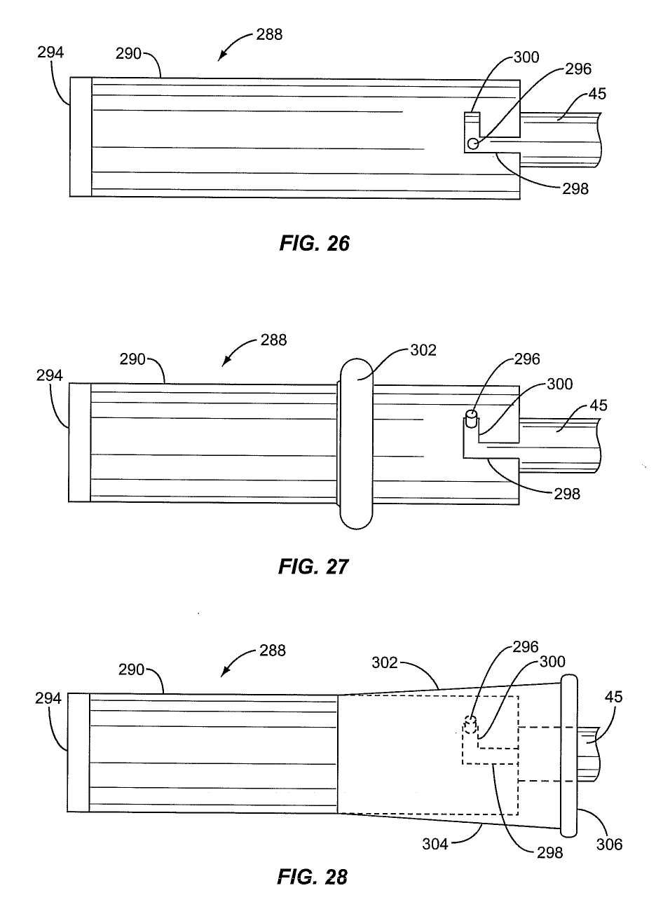

FIGS. 25A-26 illustrate an alternative embodiment of the probe tip 250 incorporating a removable sheath member 288. The removable sheath member 288 is a structure that is adapted to receive the fixed sheath 284 of the probe tip 250 to prevent the lens 282 and the fiber probe 45 from being contaminated during an endoscopic application. The removable sheath member 288 is comprised of a cylindrical-shaped wall 290 containing a hollow portion 292 that receives and surrounds the fixed sheath 284 as part of the probe tip 250. The distal end of the removable sheath member 288 contains an optical window 294. The optical window 294 provides a path for reflected light from the tissue sample to pass back to the lens 282 in the fiber probe tip 250 to capture information about the tissue. The optical window 294 also flattens the tissue to provide for an even scan and to provide greater depth resolution accuracy. The optical window 294 can be made out of any material including glass, plastic, or may comprise any other type of transparent material, including, but not limited to a membrane or other transparent material placed or stretched over the distal end of the disposable member 288. Anything that will transmit light can be used as the optical window 294.

The function of the optical window 294 is also to position the tissue relative to the lens 282 a proper distance from the tissue due to the rigid form of the cylindrical-shaped removable sheath member 288. The abutment of the optical window 294 to the tissue surface provides a fixed distance between the tissue surface and the lens 282 in the fixed sheath 284. This may be necessary to properly capture reflected light from the tissue on the lens 282. Maintaining the relationships between the tissue (via the optical window 294) and the lens 282, and between the lens 282 and the fiber probe 45 can be important in properly capturing reflected light from a tissue to analyze characteristics about its surface and/or underlying cell structures.

The optical window 294 may be perpendicular with respect to the longitudinal axis of the probe tip 250, as illustrated in FIG. 25A, or may be slanted at an angle to allow better abutment of the optical window 294 to the tissue, as illustrated in FIG. 25B. Providing an angular configuration may help avoid reflection, which can obscure reflected scattered light captured at the optical window 294. But if the angle of the optical window 294 is slight, for example, 0 to 20 degrees, and in a preferred embodiment, eight degrees, the lens 282 may still be able to properly capture the light and its angular distributions if the probe system is an angle-resolved system. If the angle of the optical window 294 will not allow the lens 282 to properly capture the angular distribution of the reflected, scattered light, the lens 282 can also be angled in the same or similar orientation to the optical window 294.

In an application of the probe tip 250 designed for a real-time f/a/LCI system, the optical window 294 is designed on the disposable removable sheath member 288 to be located approximately at the focal length of the lens 282. Providing the optical window 294 approximately one focal length away from the lens 282 allows the proper capture of the angular distributions of reflected light in the Fourier domain.

In alternative embodiments, the lens 282 may be integrated into the removable sheath member 288 as opposed to being integrated into the fixed sheath 284. Other alternative embodiments allow for different positioning of the optical window 294 relative to the lens 282.

In order to allow the removable sheath member 288 to be placed onto the probe tip 250 and removed after endoscopic application, a locking mechanism may also be included. This prevents having to wash the fixed sheath 284 after each endoscopic application since the fixed sheath 284 and the lens 282 are not exposed when protected by the removable sheath member 288. In this regard, the removable sheath member 288 is first placed onto the fixed sheath 284 prior to application. Thereafter, it may be locked into place to prevent the removable sheath member 288 from coming loose during application. After the probe tip 250 is removed from the endoscopic application, the removable sheath member 288 can be unlocked and removed for disposal. In this manner, the fixed sheath 284 and exposed lens 282, which may be one of the more expensive components of the probe tip 250, are never exposed to the tissue and do not have to be washed.

In the embodiments shown in FIGS. 25A-26, the removable sheath 288 is attached to the fiber probe 45 by sliding a locking pin 296 into a locking pin channel 298 in the removable sheath member 288. Then, the removable sheath member 288 is rotated with respect to the fixed sheath 284 to lock the removable sheath ember 288 in place. When it is desired to remove the removable sheath member 288, such as after endoscopic application, the removable sheath member 288 is rotated in the opposite direction from the locking rotation direction to allow the locking pin 296 to be removed from the locking pin channel 298. FIGS. 25A-25B illustrate the locking pin 296 engaged with the locking pin channel 298 in a cutaway view. FIG. 26 illustrates the locking pin channel 298 as it appears on the outside view of the removable sheath member 288. The locking pin channel 298 contains an angled channel portion 300 to allow the locking pin 296 to lock in place and provide resistance if the removable sheath member 288 has a force applied to it opposite from the fiber probe 45. The angled channel portion 300 is t substantially a right angle with respect to the locking pin channel 298 in the illustrated embodiment. Note, however, that the locking pin channel 298 may provide the angled channel portion 300 at other angles other than a right angle. Alternative embodiments may also provide alternative means for locking the removable sheath member 288 in place, including but not limited to a locking flange or ring mechanism.

While the removable sheath member 288 described above will prevent direct contamination of the distal face of the fiber probe 45, it is possible that fluids could penetrate through the locking pin channel 298 or come in contact with the portion of the fiber probe 45, which is not covered by the removable sheath member 288. For this reason, the probe tip 250 can be designed to additionally incorporate a deployable sterile skirt 302 which can prevent such contamination. FIGS. 27 and 28 illustrate schematics views of the skirt 302 in an initial retracted or coiled and deployed or uncoiled position, respectively.

In the illustrated embodiment, the skirt 302 is attached to the removable sheath member 288 at a point distal to the locking pin 296 and locking pin channel 298. The skirt 302 can be composed of a plastic or latex material, suitable for preventing fluid from reaching the channel or bundle. The skirt 302 may be lubricated with any type of lubricant desired before being attached to the removable sheath member 298 and/or prior to endoscopic application. Prior to deployment, the skirt 302 may be coiled or otherwise collapsed to allow for facile manipulation of the locking pin 296 within the locking pin channel 298, as illustrated in FIG. 27. Upon attachment of the removable sheath member 288 to the probe tip 250, the sterile skirt 302 can be deployed by rolling it down the removable sheath member 288 toward the proximal end. FIG. 28 shows the deployment of the sterile skirt 302, wherein the skirt provides a protective outer covering 304 of the probe tip 250 and/or the fiber probe 45. The skirt 302 may also contains a rib 306 to maintain its deployment such that the rib 306 extends beyond the diameter of the fiber probe 45. In this manner, the skirt 302 can fill any accessory channel of an endoscope to prevent contaminants from reaching the fiber probe 45.

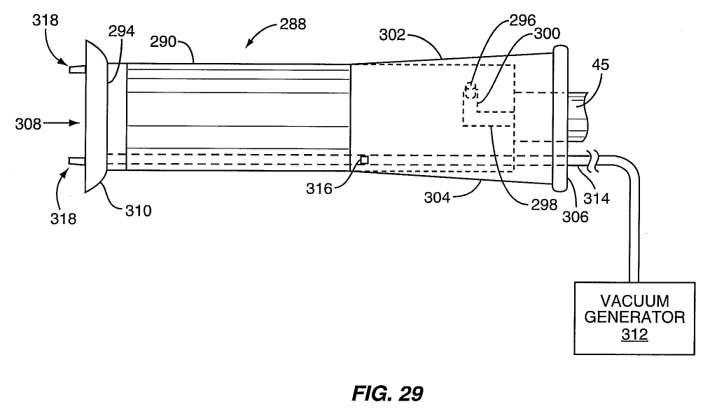

FIG. 29 illustrates an alternative embodiment of the probe tip 250 of FIGS. 27 and 28, but with additional components to assist in the abutment of the optical window 294 to the tissue to maintain the distance between the tissue and the lens 282, and the stability between the optical window 294 and the tissue. As previously discussed, it may be important to ensure the abutment of the optical window 294 to the tissue to properly receive reflected light for analysis. In this regard, a suction device 308, such as a suction cup, may also be provided on the distal end of the removable sheath member 288 to provide suction between the tissue and the optical window 294 to assist in abutment. The suction device 308 may be useful in maintaining sufficient and stable contact between the optical window 294 and the tissue. The suction device 308 may comprise a circumference-shaped material 310 that is attached to the distal end of the removable sheath member 288 and surrounds the optical window 294 so that reflected light is not obstructed. This material 310 may be any flexible material that can create a suction when pressed against a tissue surface. To provide further suction assistance, an external vacuum generator 312 may be employed and coupled to a vacuum or suction channel 314 located inside probe tip 250. The vacuum generated by the vacuum generator 312 may partially or fully assist in suction. A vacuum sensor or pressure transducer 316 may also be located within or coupled to the channel 314 to allow the detection of the pressure or vacuum at the optical window 294 to determine if proper suction is being obtained between the tissue and the optical window 294 for proper endoscope examination. The vacuum or suction channel 314 may also be used as a tissue wash if coupled to an external wash. Grasping forceps 318 may also be provided that are controllable by the person applying the probe tip 250 endoscopically to grasp the tissue to be examined to assist in the abutment of the tissue against the optical window 294.

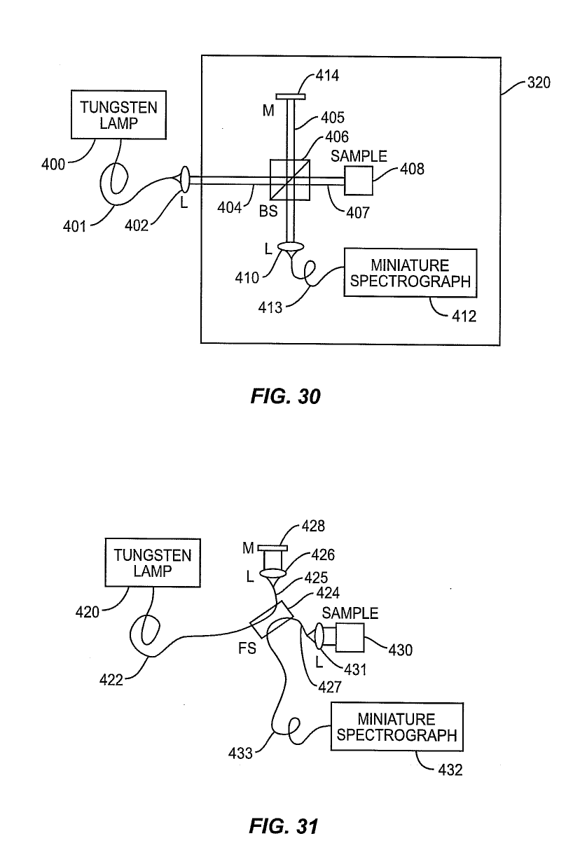

The remainder of the present application provides additional embodiments of real-time f/a/LCI systems that may be employed in the same or concomitant procedures described above. A Fourier domain optical biopsy system is possible that is not angle-resolved. These systems are referred to as fLCI systems. One exemplary embodiment of a fLCI system 320 is shown in FIG. 30. In this regard, white light from a Tungsten light source 400 (e.g., 6.5 W, Ocean Optics™) is coupled into a multimode fiber 401 (e.g., 200 μm core diameter). The output of the fiber 401 is collimated by an achromatic lens 402 to produce a beam 404 (e.g., a pencil beam 5 mm in diameter). The beam 404 is then forwarded to the fLCI system 320.

This illumination scheme achieves Kohler illumination in that the fiber acts as a field stop, resulting in the proper alignment of incident or illuminating light and thereby achieving critical illumination of the sample. In the fLCI system 320, the white light beam is split by the beamsplitter 406 (BS) into a reference beam 405 and an input beam 407 to the sample 408. The light scattered by the sample 408 is recombined at the BS 406 with light reflected by the reference mirror 414 (M).

The reference beam 405 in conjunction with the reference mirror 414 forms a portion of a reference arm that receives a first reference light and outputs a second reference light. The input beam 407 and the sample 408 form a portion of a sample arm that receives a first sample light and outputs a second sample light.

Those skilled in the art will appreciate that the light beam can be split into a plurality of reference beams and input beams (e.g., N reference beams and N input beams) without departing from the spirit and scope of the present invention. Further, the splitting of the beams may be accomplished with a beamsplitter or a fiber splitter in the case of an optical fiber implementation of an exemplary embodiment of the present invention.

In the exemplary embodiment of the present invention shown in FIG. 30, the combined beam is coupled into a multimode fiber 413 by an aspheric lens 410. Again, other coupling mechanisms or lens types and configurations may be used without departing from the spirit and scope of the present invention. The output of the fiber coincides with the input slit of a miniature spectrograph 412 (e.g., USB2000, Ocean Optics™), where the light is spectrally dispersed and detected.

The detected signal is linearly related to the intensity as a function of wavelength I(λ), which can be related to the signal and reference fields (Eₛ, Eᵣ) as:

[symbol]

I(λ) [symbol] = [symbol] Eₛ(λ)|² [symbol] + [symbol] Eᵣ(λ)|² [symbol] +2Re [symbol] Eₛ(λ)E*ᵣ(λ) [symbol] cos φ (8)

where φ is the phase difference between the two fields and < . . . > denotes an ensemble average.

The interference term is extracted by measuring the intensity of the signal and reference beams independently and subtracting them from the total intensity.

The axial spatial cross-correlation function, Γ_{SR}(z) between the sample and reference fields is obtained by resealing the wavelength spectrum into a wavenumber (k=2π/λ) spectrum then Fourier transforming:

Γ_{SR}(z)=∫dke^{ikz} [symbol] Eₛ(k)E*ᵣ(k) [symbol] cos φ (9)

This term is labeled as an axial spatial cross-correlation as it is related to the temporal or longitudinal coherence of the two fields.

Another exemplary embodiment of an fLCI scheme is shown in FIG. 31. In this exemplary embodiment, fiber optic cable is used to connect the various components. Those skilled in the art will appreciate that other optical coupling mechanisms, or combinations thereof, may be used to connect the components without departing from the spirit and scope of the present invention.

In FIG. 31, white light from a Tungsten light source 420 is coupled into a multimode fiber 422 and the white light beam in the multimode fiber is split by the fiber splitter (FS) 424 into a reference fiber 425 and a sample fiber 427 to the sample 430. The fiber splitter 424 is used to split light from one optical fiber source into multiple sources.

The reference light in reference fiber 425, in conjunction with a lens 426 (preferably an aspheric lens) and the reference mirror 428, forms a portion of a reference arm that receives a first reference light and outputs a second reference light. Specifically, reference light in reference fiber 425 is directed to the reference mirror 428 by lens 426, and the reference light reflected by the reference mirror 428 (second reference light) is coupled back into the reference fiber 425 with lens 426. The sample light in sample fiber 427 and the sample 430 form a portion of a sample arm that receives a first sample light and outputs a second sample light. Specifically, sample light in sample fiber 427 is directed to the sample 430 by lens 434 (preferably as aspheric lens), and at least a portion of the sample light scattered by the sample 430 is coupled into the sample fiber 427 by lens 431. In the exemplary embodiment shown in FIG. 31, the sample 430 is preferably spaced from lens 431 by a distance approximately equal to the focal length of lens 431.

At least a portion of the reflected reference light in reference fiber 425 and at least a portion of the scattered sample light on sample fiber 427 are coupled into a detector fiber 433 by the FS 424. The output of detector fiber 433 coincides with the input of a miniature spectrograph 432, where the light is spectrally dispersed and detected.

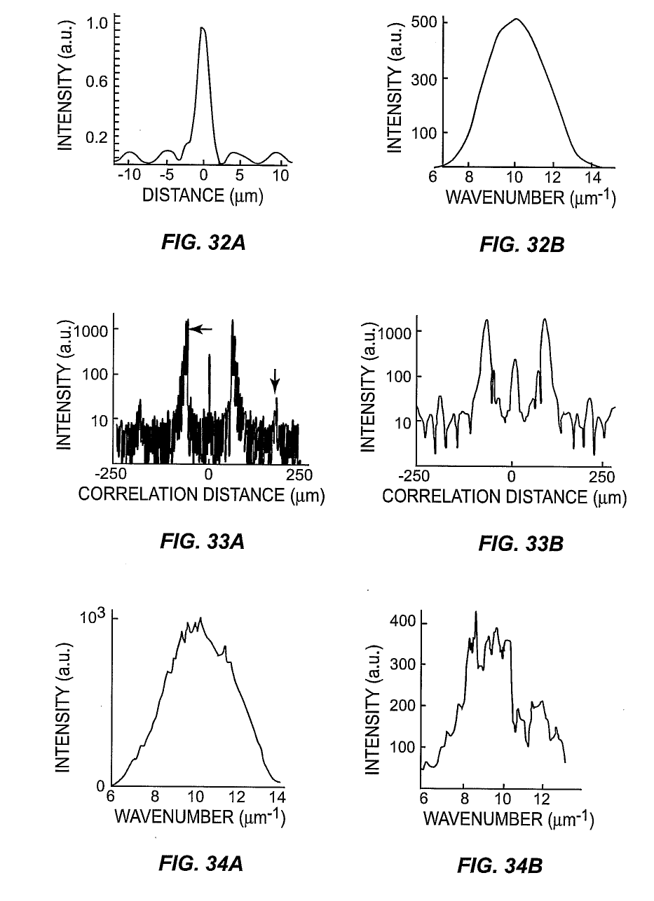

FIGS. 32A and 32B illustrate some of the properties of a white light source. FIG. 32A illustrates an autocorrelation function showing a coherence length (l_{C}=1.2 μm). FIG. 32A shows the cross-correlation between the signal and reference fields when the sample is a mirror, and this mirror is identical to the reference mirror (M). In this exemplary scenario, the fields are identical and the autocorrelation is given by the transform of the incident field spectrum, modeled as a Gaussian spectrum with center wavenumber kₒ=10.3 μm⁻¹ and 1/e width Δk_{1/e}=2.04 μm⁻¹ (FIG. 32B).

FIG. 32B shows an exemplary spectrum of light source that can be used in accordance with the present invention.

From this autocorrelation, the coherence length of the field, l_{c}=1.21 μm is determined. This is slightly larger than the calculated width of l_{c}=2/Δk_{1/c}=0.98 μm, with any discrepancy most likely attributed to uncompensated dispersion effects. Note that rescaling the field into wavenumber space is a nonlinear process which can skew the spectrum if not properly executed.

In data processing, a fitting algorithm is applied (e.g., a cubic spline fit) to the rescaled wavenumber spectrum and then resampled (e.g., resample with even spacing). The resampled spectrum is then Fourier transformed to yield the spatial correlation of the sample. Those skilled in the art will appreciate that other frequency-based algorithms or combinations of algorithms can be used in place of the Fourier transform to yield spatial correlation. One example of a software tool that can be used to accomplish this processing in real time or near real time is to use LabView™ software.

In one exemplary embodiment of the present invention, the sample consists of a glass coverslip (e.g., thickness, d˜200 μm) with polystyrene beads which have been dried from suspension onto the back surface (1.55 μm mean diameter, 3% variance). Thus, the field scattered by the sample can be expressed as:

Eₛ(k)=E_{front}(k)e^{ik}^{^{δ}}^{z}+E_{back}(k)e^{ik(}^{^{δ}}^{z+nd)} (10)

In Equation 10, E_{front} and E_{back} denote the field scattered by the front and back surfaces of the coverslip, and δz is the difference between the path length of the reference beam and that of the light reflected from the front surface and n the index of refraction of the glass. The effect of the microspheres will appear in the E_{back} term as the beads are small and attached closely to the back surface. Upon substituting Equation 10 into Equation 9, a two peak distribution with the width of the peaks given by the coherence length of the source is obtained.

In order to obtain spectroscopic information, a Gaussian window is applied to the interference term before performing the Fourier transform operation. Those skilled in the art will appreciate that other probabilistic windowing methodologies may be applied without departing from the spirit and scope of the invention. This makes it possible to recover spectral information about light scattered at a particular depth.

The windowed interference term takes the form:

[symbol]

Eₛ(k)E*ᵣ(k) [symbol] exp[−((k−k_{w})/Δk_{w})²]. (1)

The proper sizing of a windowed interference term can facilitate the processing operation. For example, by selecting a relatively narrow window (Δk_{w} small) compared to the features of Eₛ and Eₖ, we effectively obtain <Es(kw)E*r(kw)>. In processing the data below, we use Δk_{w}=0.12 μm⁻¹ which degrades the coherence length by a factor of 16.7. This exemplary window setting enables the scattering at 50 different wavenumbers over the 6 μm⁻¹ span of usable spectrum.

In FIGS. 33A and 33B, an axial spatial cross-correlation function for a coverslip sample is shown according to one embodiment of the invention. FIGS. 33A and 33B show the depth-resolved cross-correlation reflection profiles of the coverslip sample before and after the processing operations. In FIG. 33A, a high resolution scan with arrows indicating a peak corresponding to each glass surface is shown. In FIG. 33B, a low resolution scan obtained from the scan in FIG. 33A is shown by using a Gaussian window.

Note that the correlation function is symmetric about z=0, resulting in a superposed mirror image of the scan. Since these are represented as cross-correlation functions, the plots are symmetric about z=0. Thus, the front surface reflection for z>0 is paired with the back surface reflection for z<0, and vice versa.

In FIG. 33A, the reflection from the coverslip introduces dispersion relative to the reflection from the reference arm, generating multiple peaks in the reflection profile. When the spectroscopic window is applied, only a single peak is seen for each surface, however several dropouts appear due to aliasing of the signal.

To obtain the spectrum of the scattered light, we repeatedly apply the Gaussian window and increase the center wavenumber by 0.12 μm⁻¹ between successive applications. As mentioned above, Δk_{w}=0.12 μm⁻¹ is used to degrade the coherence length by a factor of 16.7. This results in the generation of a spectroscopic depth-resolved reflection profile.

FIGS. 34A and 34B show the spectrum obtained for light scattered from the front (a) and back (b) surfaces of a coverglass sample respectively, when no microspheres are present. The reflection from the front surface appears as a slightly modulated version of the source spectrum. The spectrum of the reflection from the rear surface however has been significantly modified. Thus in Equation 10, we now take E_{front}(k)=Eₛ(k) and E_{back}(k)=T(k)Eₛ(k), where T(k) represents the transmission through the coverslip.

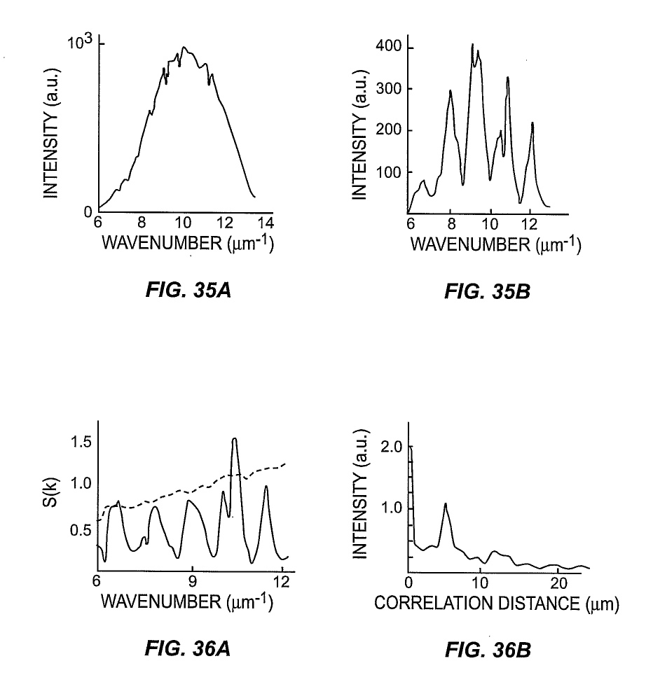

In FIGS. 35A and 35B, illustrate the spectra for light scattering obtained for front (a) and back (b) surfaces of a coverglass sample when microspheres are present on the back surface of the coverslip. It can be seen that the reflected spectrum from the front surface has not changed significantly, as expected. However, the spectrum for the back surface is now modulated. The scattering properties S(k) of the microspheres can be examined by writing the scattered field as Eₛₚₕₑᵣₑₛ(k)=S(k)T(k)Eₛ(k) and taking the ratio Eₛₚₕₑᵣₑₛ(k)/E_{back}(k)=S(k), which is shown as a solid line in FIG. 36A. It can be seen from this ratio that the microspheres induce a periodic modulation of the spectrum.

In FIG. 36A, a ratio of the spectra found in FIGS. 34A-35B is shown. This illustrates the scattering efficiency of spheres for front (represented by the dashed line) and back (represented by the solid line) surface reflections. In FIG. 36B, a correlation function obtained from ratio of back surface reflections is shown. The peak occurs at the round trip optical path through individual microspheres, permitting the size of the spheres to be determined with sub-wavelength accuracy.

For comparison, the same ratio for the front surface reflections (dashed line in FIG. 35A) shows only a small linear variation. Taking the Fourier transform of S(k) yields a clear correlation peak (FIG. 36B), at a physical distance of z=5.24 μm. This can be related to the optical path length through the sphere by z=2 nl with the index of the microspheres n=1.59. The diameter of the microspheres to be l=1.65 μm+/−0.33 μm, with the uncertainty given by the correlation pixel size. Thus with fLCI, we are able to determine the size of the microspheres with sub-wavelength accuracy, even exceeding the resolution achievable with this white light source and related art LCI imaging.

There are many applications of the various exemplary embodiments of the present invention. One exemplary application of fLCI is in determining the size of cell organelles, in particular the cell nucleus, in epithelial tissues. In biological media, for example, the relative refractive indices are lower for organelles compared to microspheres and thus, smaller scattering signals are expected. The use of a higher power light source will permit the smaller signals to be detected. Other examples include detection of sub-surface defects in manufactured parts, including fabricated integrated circuits, detection of airborne aerosols, such as nerve agents or biotoxins, and detection of exposure to such aerosols by examining epithelial tissues within the respiratory tract.

Additionally, the larger the size of the nucleus (compared to the microspheres in this experiment), the higher the frequency modulation of the spectrum. Those skilled in the art will appreciate that higher frequency oscillations are detected at a lower efficiency in Fourier transform biopsy techniques. Therefore, in order to detect these higher frequency oscillations, a higher resolution spectrograph is used.

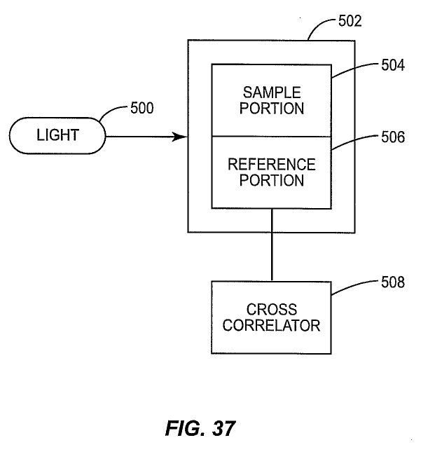

FIG. 37 illustrates a generalized embodiment of the fLCI system shown in FIG. 30 and discussed in greater detail above. In FIG. 37, a light source 500 (e.g., a multi-wavelength light) is coupled into an fLCI system 502. Within the fLCI system 502, a sample portion 504 and a reference portion 506 are located. The sample portion 504 includes a light beam and light scattered from a sample. For example, the sample portion 504 may include a sample holder, a free space optical arm, or an optical fiber. The reference portion 506 includes a light beam and light that is reflected from a reference. For example, the reference portion 506 may include an optical mirror. A cross-correlator 508 receives and cross-correlates light from the sample with light from the reference.

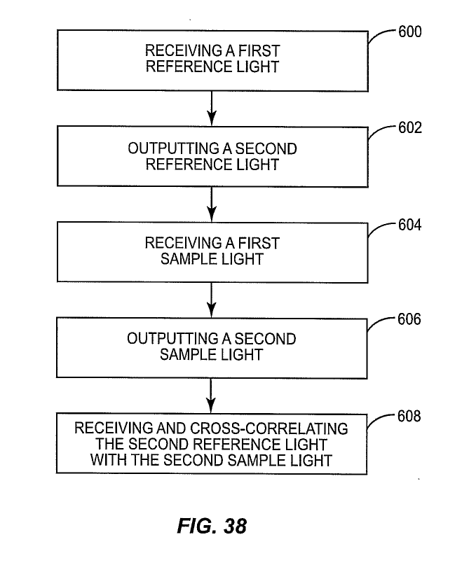

FIG. 38 illustrates another exemplary embodiment of the present invention. In FIG. 38, a method is disclosed where a first reference light is received (block 600) and a second reference light is output 502. A first sample light is received (block 604) and a second sample light is output (block 606). The second sample light contains light scattered from a sample when at least a portion of the first sample light is scattered from a sample. The second reference light with the second sample light are received and cross-correlated (block 608).

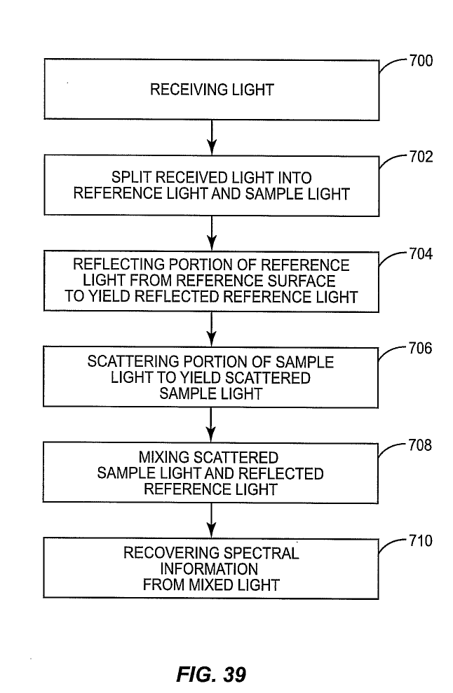

FIG. 39 illustrates another exemplary embodiment of the present invention. In FIG. 39, a method is disclosed where light is received (block 700 from a sample that has been illuminated. At least a portion of the light is split into reference light and sample light (block 702). At least a portion of said reference light is reflected from a reference surface to yield reflected reference light (block 704). At least a portion of the sample light is scattered from a sample to yield scattered sample light (block 706). The scattered sample light and the reflected reference light are mixed (block 708). Spectral information is recovered about the scattered sample light (block 710).

Embodiments disclosed herein also involve new low-coherence interferometry (LCI) techniques which enable acquisition of structural and depth information regarding a sample of interest at rapid rates. A sample can be tissue or any other cellular-based structure. The acquisition rate is sufficiently rapid to make in vivo applications feasible. Measuring cellular morphology in tissues and in vivo as well as diagnosing intraepithelial neoplasia and assessing the efficacy of chemopreventive and chemotherapeutic agents are possible applications. Prospectively grading tissue samples without tissue processing is also possible, demonstrating the potential of the technique as a biomedical diagnostic.

In one embodiment, a “swept-source” (SS) light source is used in LCI to obtain structural and depth information about a sample. The swept-source light source is used to generate a reference signal and a signal directed towards a sample. Light scattered from the sample is returned as a result and mixed with the reference signal to achieve interference and thus provide structural and depth-resolved information regarding the sample. With a “swept-source” light source, the light source is controlled or varied to sweep the center wavelength of a narrow band of emitted light over a given range of wavelengths, thus synthesizing a broad band source. Because the light is emitted in particular wavelengths or narrower ranges of wavelengths during emission, scattered light returned from the sample is known to be in response to a particular wavelength or range of wavelengths. Thus, the returned scattered light is spectrally-resolved and depth-resolved, because the returned light is in response to the light source emitted light over a narrow spectral range. This is opposed to a wider or light source that generates all wavelengths of light in one light emission in time, wherein the returned scattered light from the sample contains scattered light at a broad range of wavelengths. In this instance, a spectrometer is used to spectrally-resolve the returned scattered light. However, when using a swept-source light source, the series of returned scattered lights from the sample at each wavelength are already in the spectral domain to provide spectrally-resolved information about the sample. The spectrally-resolved information about the sample can be detected.

Another embodiment involves using a swept-source light source in angle-resolved low-coherence interferometry (a/LCI), referred to herein as “swept-source Fourier domain a/LCI,” or “SS a/LCI.” The data acquisition time for SS a/LCI can be less than one second, a threshold which is desirable for acquiring data from in vivo tissues. The swept-source light source is employed to generate a reference signal and a signal directed towards a sample over the swept range of wavelengths or ranges of wavelengths. The light is either directed to strike the sample at an angle, or the light source or another component in the system (e.g., a lens) is moved to direct light onto the sample at an angle or plurality of angles (i.e., two or more angles), which may include a multitude of angles (i.e., more than two angles). This causes a set of scattered light to be returned from the sample at a plurality of angles, thereby representing spectrally-resolved and angle-resolved (also referred to herein as “spectral and angle-resolved”) scattered information about the sample from a plurality of points on the sample. The spectral and angle-resolved scattered information about the sample can be detected. This SS a/LCI embodiment can also use the Fourier domain concept to acquire depth-resolved information. It has recently been shown that improvements in signal-to-noise ratio, and commensurate reductions in data acquisition time are possible by recording the depth scan in the Fourier (or spectral) domain. In this embodiment, the SS a/LCI system can combine the Fourier domain concept with the use of a swept-source light source, such as a swept-source laser, and a detector, such as a line scan array or camera, to record the angular distribution of returned scattered light from the sample in parallel and the frequency distribution in time.

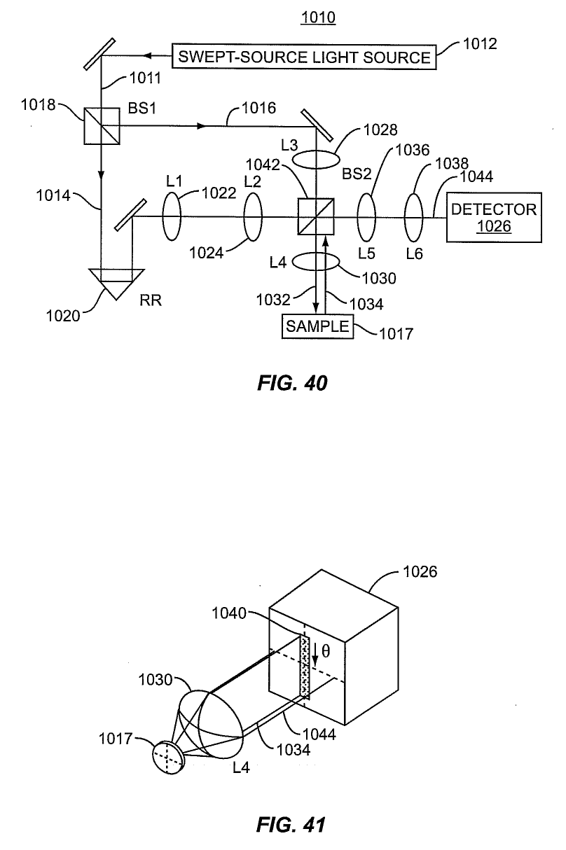

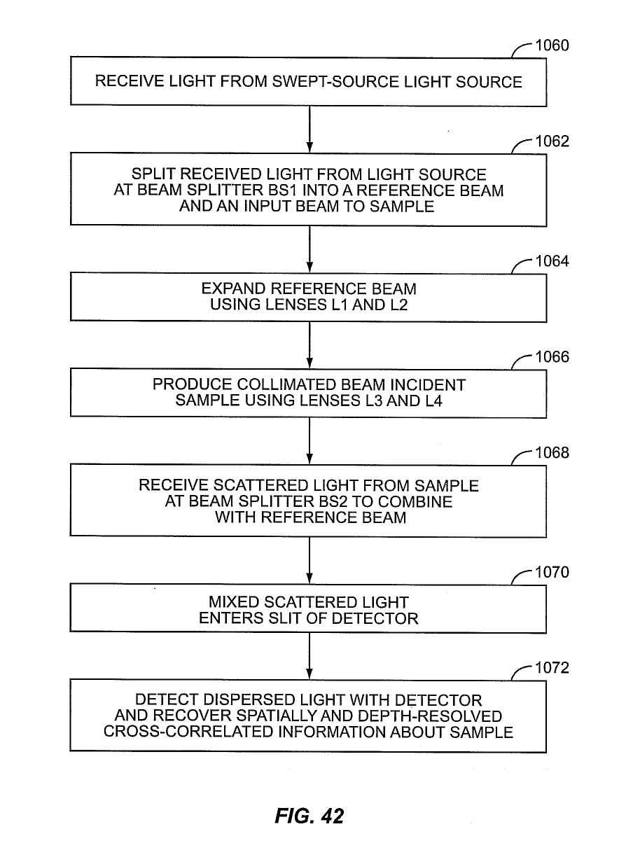

FIGS. 40 and 41 illustrate an example of an SS a/LCI system 1010 according to one embodiment of the invention. The SS a/LCI apparatus and system in FIG. 40 may be based on a modified Mach-Zehnder interferometer. The discussion of the SS a/LCI system 1010 in FIGS. 40 and 41 will be discussed in conjunction with the steps performed in the system 1010 provided in the flowchart of FIG. 42. As illustrated in FIG. 40, light 1011 from a swept-source light source 1012 in the form of a swept-source laser 1012 is generated. The light from the swept-source light source 1012 is received (block 60, FIG. 42) split into a reference beam 1014 and an input beam 1016 to a sample 1017 by beam splitter (BS1) 1018 (block 62, FIG. 42). The path length of the reference beam 1014 is set by adjusting retroreflector (RR) 1020, but remains fixed during measurement. The reference beam 1014 is expanded using lenses (L1) 1022 and (L2) 1024 (block 64, FIG. 42) to create illumination which is uniform and collimated upon reaching a detector device 1026, which may be a line scan array or camera as examples.

Lenses (L3) 1028 and (L4) 1030 are arranged to produce a collimated pencil beam 1032 incident on the sample 1017 (block 66, FIG. 42). By displacing lens (L4) 1030 vertically relative to lens (L3) 1028, the input beam 1032 is made to strike the sample 1017 at an angle relative to the optical axis. In this embodiment, the input beam 1032 strikes the sample 1017 at an angle of approximately 0.10 radians; however, the invention is not limited to any particular angle. This arrangement allows the full angular aperture of lens (L4) 1030 to be used to collect returned scattered light 1034 from the sample 1017.

The light scattered by the sample 1017 is collected by lens (L4) 1030 (block 1068, FIG. 42) and relayed by a 4f imaging system, via lenses (L5) 1036 and (L6) 1038, such that the Fourier plane of lens (L4) 1030 is reproduced in phase and amplitude at a slit 1040, as illustrated in FIG. 41 (block 1070, FIG. 42). The scattered light 1034 is mixed with the reference beam 1014 at beam splitter (BS2) 1042 with combined beams 1044 falling upon the detector device 1026. The combined beams 1044 are processed to recover depth-resolved spatial cross-correlated information about the sample 1017 (block 1072, FIG. 42).

In this embodiment, the detector device 1026 is a one-dimensional detection device in the form of a line scan array, which is comprised of a plurality of detectors. This allows the detector device 1026 to receive light at the plurality of scatterer angles from the sample 1017 and mixed with the reference beam 1014 at the same time or essentially the same time to receive spectral information about the sample 1017. Providing the line scan array 1026 allows detection of the angular distribution of the combined beams 1044, or said another way, at multiple scatter angles. Each detector in the detector device 1026 receives scattered light from the sample 1017 at a given angle at the same time or essentially the same time.

Because the emitted light from the swept-source light source 1012 is broken up into particular wavelengths or narrower ranges of wavelengths during emission, returned scattered light 1034 from the sample 1017 is known to be in response to a particular wavelength or range of wavelengths. Thus, the returned scattered light 1034 is spectrally-resolved, because the returned scattered light 1034 is in response to the light source emitted light over a spectral domain. This is opposed to a wider or broadband light source that generates all wavelengths of light in one light emission at the same time, wherein the returned scattered light from the sample contains scattered light at all wavelengths. In this instance, a spectrometer is used to spectrally-resolve the returned scattered light. However, when using the swept-source light source 1012, the series of returned scattered light 1034 from the sample 1017 at each wavelength is already in the spectral domain to provide spectrally-resolved information about the sample.

FIG. 41 illustrates an example of the distribution of scattering angles across the dimension of the front of a line scan array 1026. The combined beams or detected signal 1044 detected by the detector device 1026 is a function of vertical position on the line scan array, y, and wavelength, λ, which is a function of time as the swept-source light source 1012 is swept across its wavelength range. The detected signal 1044 at pixel m and time t can be related to the scattered light 1034 and reference beam 1014 (Eₛ, Eᵣ) as:

I(λₘ,yₙ)= [symbol] |Eᵣ(λₘ,yₙ)|² [symbol] + [symbol] Eₛ(λₘ,yₙ)|² [symbol] +2Re [symbol] Eₛ(λₘ,yₙ)E*ᵣ(λₘ,yₙ) [symbol] cos φ (12)

where Φ is the phase difference between the two fields and [symbol] . . . [symbol] denotes an ensemble average in time. The interference term is extracted by measuring the intensity of the scattered light 1034 and reference beam 1014 independently and subtracting them from the total intensity. In one method of obtaining depth-resolved information about the sample 1017, the wavelength spectrum at each scattering angle is interpolated into a wavenumber (k=2π/λ) spectrum and Fourier transformed to give a spatial cross correlation, Ε_{SR}(z) for each vertical pixel yₙ:

Ε_{SR}(z,yₙ)=∫dke^{ikz} [symbol] Eₛ(k,yₙ)E*ᵣ(k,yₙ) [symbol] cos φ (13)

The reference field takes the form:

Eᵣ(k)=Eₒexp[−((k−kₒ)/Δk)²]exp[−((y−yₒ)/Δy)²]exp[ikΔl] (14)

where kₒ (yₒ and Δk (Δy) represent the center and width of the Gaussian wavevector (spatial) distribution and Δl is the selected path length difference. The scattered sample field takes the form:

Eₛ(k,θ)=ΣⱼEₒexp[−((k−kₒ)/Δk)²]exp[iklⱼ]Sⱼ(k,θ) (15)

where Sⱼ represents the amplitude distribution of the scattering originating from the jth interface, located at depth lⱼ. The angular distribution of the scattered sample field is converted into a position distribution in the Fourier image plane of lens (L4) 1030 through the relationship y=f₄θ. For the exemplary pixel size of the line scan array 1026 of eight (8) to twelve (12) micrometers (μm), this yields an angular resolution of 0.00028 to 0.00034 mradians and an expected angular range of 286 to 430 mradians for a 1024 element array. Inserting Equations (14) and (15) into Equation (13) and noting the uniformity of the reference field (Δy>>camera height) yields the spatial cross correlation at the nth vertical position on the detector:

$${Γ}_{SR} ( z , {y}_{n} ) = {\sum }_{j} \int d k {\vert {E}_{o} \vert}^{2} exp [ - 2 {( ( k - {k}_{o} ) / Δ k )}^{2} ] exp [ i k ( z - Δ l + {l}_{j} ) ] \times {S}_{j} ( k , {θ}_{n} = {y}_{n} / {f}_{4} ) cos φ \qquad ( 16 )$$

Evaluating this equation for a single interface yields:

Ε_{SR}(z,yₙ)=|Eₒ|²exp[−((z−Δl+lⱼ)Δk)²/8]Sⱼ(kₒ,θₙ=yₙ/f₄)cos φ (17)

Here, it is assumed that the scattering amplitude S does not vary appreciably over the bandwidth of the source. This expression shows obtaining a depth-resolved profile of the scattering distribution with each vertical pixel corresponding to a scattering angle. The techniques described in U.S. patent application Ser. No. 11/548,468 entitled “Systems and Methods for Endoscopic Angle-Resolved Low Coherence Interferometry,” which is incorporated herein by reference in its entirety, may be used for obtaining structural and depth-resolved information regarding scattered light from a sample.

To obtain the same or similar data set as is obtained from a single frame capture from an imaging spectrometer using a broadband light source, the SS a/LCI apparatus and system 1010 can capture a series of data acquisitions from the line scan array 1026 at each wavelength and combine them. In this embodiment, the data acquisition rate of the line scan arrays 1026 is less than the sweep rate of the swept-source light source 1012. If one were to assume that 1000 wavelength (frequency) points are needed (and thus points in time for the swept-source), ten (10) to twenty (20) data acquisitions of scattered information from the sample 1017 may be recovered per second using a line scan array. For example, this scenario could yield a time per acquisition of 50 to 100 milliseconds, which is satisfactory for clinical and commercial viability.

Line scan arrays and camera detector devices are widely available for both the visible and the near infrared wavelengths. Visible line scan arrays can operate from approximately ˜400 nm to ˜900 nm, for example, and may be based on silicon technology. Near infrared line scan arrays may operate from approximately ˜900 nm to ˜1700 nm or further. Table 2 below gives some typical specifications from several manufacturers as examples.

TABLE 2
Examples of Line Scan Arrays

| | | | | Readout rate |
|---|---|---|---|---|
| | | | Pixel size | (1000 lines/ |
| Manufacturer | λ range (nm) | Pixel number | (μm) | second) |
| Atmel | 400-950 | 512-4096 | 7-14 | 14 to 100 |
| Hamamatsu | 400-950 | 128-1024 | 25-50 | 2 to 20 |
| Fairchild | 400-850 | 2048 | 7 | 38 |
| Imaging | | | | |
| Hamamatsu | 900-1550 | 256-512 | 25-50 | 1 to 10 |
| Sensors | 900-1700 | 128-1024 | 25-50 | 4 to 20 |
| Unlimited | | | | |

As previously discussed above, a swept-source laser may be employed as the swept-source light source 1012. Some examples are provided in Table 3 below.

TABLE 3
Examples of Swept-source Light Sources (Swept-source Lasers)

| | | | Sweep rate | |
|---|---|---|---|---|
| Manufac- | | | (1000 sweeps/ | Power |
| turer | Center λ nm | Δλ nm | second) | (mW) |
| Thorlabs | 1325 | 150 | 17 | 12 |
| Micron | 1060, 1310, 1550 | 50, 110, 150 | 8 | 5, 20, 20 |
| Optics | | | | |
| Santec | 1310 | 110 | 20 | 3 |

Faster acquisition times are possible. Swept-source light sources at shorter wavelengths will allow use of a high speed detector 1026, such as silicon detectors for example. For example, some Atmel® silicon-based cameras can achieve 100,000 lines per second, potentially allowing 100 data point acquisitions per second or 10 milliseconds per acquisition. Alternately, as another example, the line scan array 1026 may be based on InGaAs technology and may be faster, reaching readout rates of 50,000 to 100,000 lines per second and thus reducing the acquisition time to 10 milliseconds. It is expected that the sweep rate, power, wavelength range, and other performance characteristics of the swept-source light sources can enable high performance versions of the a/LCI apparatuses and systems, including the SS a/LCI apparatus and system 1010 of FIGS. 40 and 41.

In addition to obtaining depth-resolved information about the sample 1017, the scattering distribution data (i.e., a/LCI data) obtained from the sample 1017 using the disclosed data acquisition scheme can also be used to make a size determination of the nucleus using the Mie theory, as previously discussed. A filtered curve is determined using the scattered data. Comparison of the filtered scattering distribution curve (i.e., a representation of the scattered data) to the prediction of Mie theory enables a size determination to be made.

In order to fit the scattered data to Mie theory, the a/LCI signals are processed to extract the oscillatory component which is characteristic of the nucleus size. The smoothed data is fit to a low-order polynomial (2nd order is typically used but higher order polynomials, such as 4^{th} order, may also be used), which is then subtracted from the distribution to remove the background trend. The resulting oscillatory component can then be compared to a database of theoretical predictions obtained using Mie theory from which the slowly varying features were similarly removed for analysis.

A direct comparison between the filtered a/LCI data and Mie theory data may not be possible, as the Chi-squared fitting algorithm tends to match the background slope rather than the characteristic oscillations. The calculated theoretical predictions include a Gaussian distribution of sizes characterized by a mean diameter (d) and standard deviation as well as a distribution of wavelengths to accurately model the broad bandwidth source.

The best fit can be determined by minimizing the Chi-squared between the data and Mie theory, yielding a size of 10.2.+/−.1.7 μm, in excellent agreement with the true size. The measurement error is larger than the variance of the bead size, most likely due to the limited range of angles recorded in the measurement.

As an alternative to processing the a/LCI data and comparing to Mie theory, there are several other approaches which could yield diagnostic information. These include analyzing the angular data using a Fourier transform to identify periodic oscillations characteristic of cell nuclei. The periodic oscillations can be correlated with nuclear size and thus will possess diagnostic value. Another approach to analyzing a/LCI data is to compare the data to a database of angular scattering distributions generated with finite element method (FEM) or T-Matrix calculations. Such calculations offer superior analysis as they are not subject to the same limitations as Mie theory. For example, FEM or T-Matrix calculations can model non-spherical scatterers and scatterers with inclusions while Mie theory can only model homogenous spheres. Other techniques are described in U.S. Pat. No. 7,102,758 entitled “Fourier Domain Low-Coherence Interferometry for Light Scattering Spectroscopy Apparatus and Method,” which is incorporated herein by reference in its entirety.

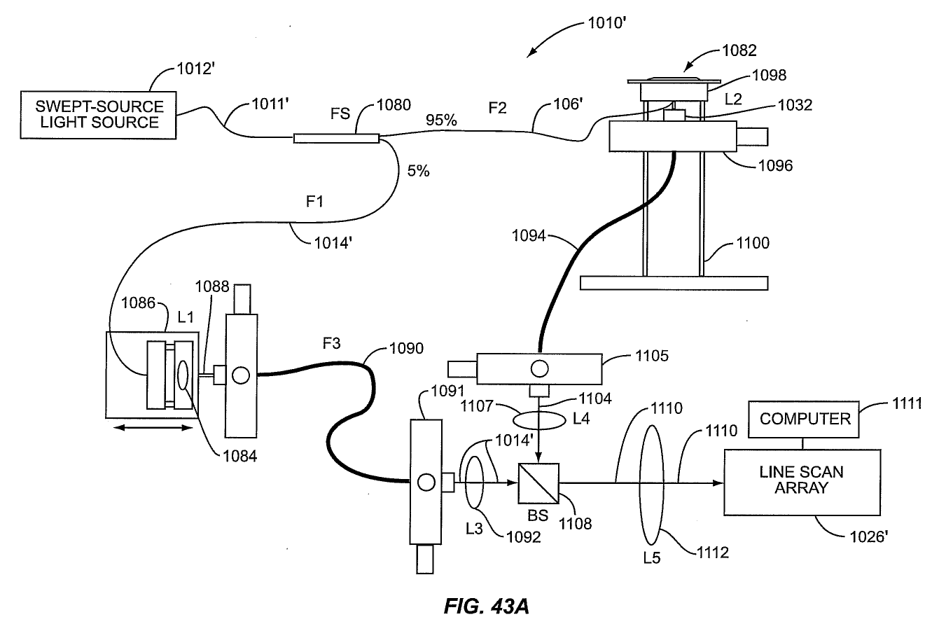

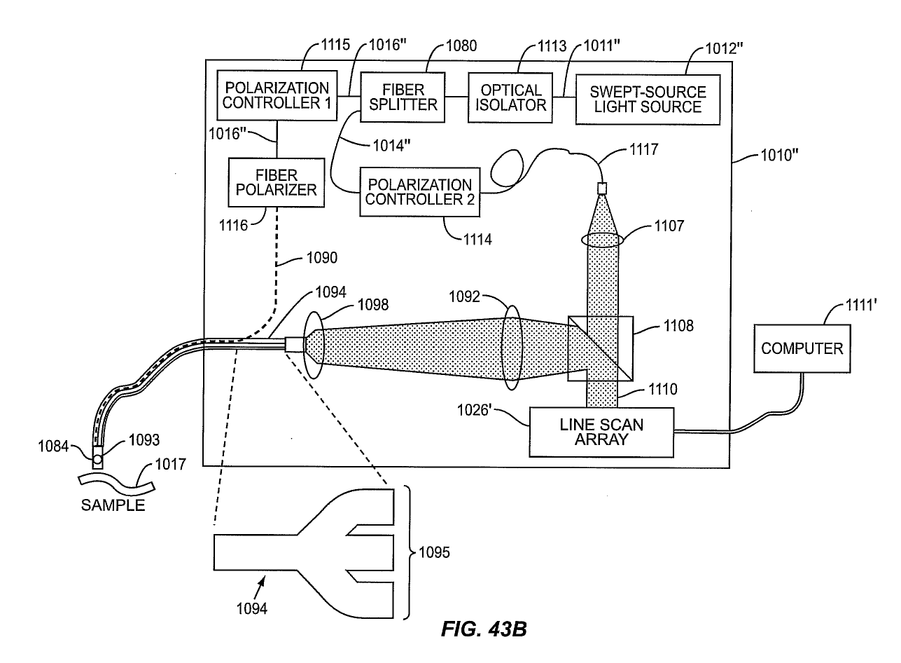

In another embodiment of the invention, an SS a/LCI apparatus and system can be provided, including for endoscopic applications, by using optical fibers to deliver and collect light from the sample of interest. These alternative embodiments are illustrated in FIGS. 43A and 43B. The fiber optic portion of the system is nearly identical, and the system changes consist of a swept-source light source 1012′ in place of the superluminescent diode, a line scan array (or camera) in place of the imaging spectrometer, and modification to the data processing to aggregate multiple acquisitions from the line scan array. The angular distribution of the returned scattered light from the sample is captured by locating the distal end of a fiber bundle in a conjugate Fourier transform plane of the sample using a collecting lens. This angular distribution is then conveyed to the distal end of the fiber bundle where it is imaged using a 4f system onto the line scan array. A beam splitter is used to overlap the scattered sample field with a reference field prior to the line scan array so that low-coherence interferometry can also be used to obtain depth-resolved measurements.

Turning now to FIG. 43A, a fiber optic SS a/LCI system 1010′ is illustrated. A similar fiber optic SS a/LCI system 1010′ is also illustrated in FIG. 43B. The fiber optic SS a/LCI system 1010′ can make use of the Fourier transform properties of a lens. This property states that when an object is placed in the front focal plane of a lens, the image at the conjugate image plane is the Fourier transform of that object. The Fourier transform of a spatial distribution (object or image) is given by the distribution of spatial frequencies, which is the representation of the image's information content in terms of cycles per mm. In an optical image of elastically scattered light, the wavelength retains its fixed, original value and the spatial frequency representation is simply a scaled version of the angular distribution of scattered light. In the fiber optic SS a/LCI system 1010′, the angular distribution of scattered light from the sample is captured by locating the distal end of the fiber bundle in a conjugate Fourier transform plane of the sample using a collecting lens.

Turning to FIG. 43A, light 1011′ from a swept-source light source 1012′ is split into a reference beam 1014′ and an input beam 1016′ using a fiber splitter (FS) 1080. A splitter ratio of 20:1 may be chosen in one embodiment to direct more power to a sample (not shown) via a signal arm 1082 as the returned scattered light 1034′ from the sample is typically only a small fraction of the incident power. Light in the reference beam 1014′ emerges from fiber (F1) and is collimated by lens (L1) 1084 which is mounted on a translation stage 1086 to allow gross alignment of the reference arm path length. This path length is not scanned during operation but may be varied during alignment. A collimated beam 1088 is arranged to be equal in dimension to the end 1091 of fiber bundle (F3) 1090 so that the collimated beam 88 illuminates all fibers in the fiber bundle (F3) 1090 with equal intensity. The reference beam 1014′ emerging from the distal tip of the fiber bundle (F3) 1090 is collimated with lens (L3) 1092 in order to overlap with the scattered sample field conveyed by fiber bundle (F4) 1094 having a fiber breakout 1095 to capture the returned scattered light form the sample 1017 at a plurality of angles at the same time. In an alternative embodiment, light emerging from fiber (F1) is collimated then expanded using a lens system to produce a broad beam.

The scattered sample field is detected using a coherent fiber bundle. The scattered sample field is generated using light in the signal arm 1082 which is directed toward the sample of interest using lens (L2) 1098. As with the free space system, lens (L2) 1098 is displaced laterally from the center of single-mode fiber (F2) such that a collimated beam is produced which is traveling at an angle relative to the optical axis. The fact that the incident beam strikes the sample at an oblique angle is essential in separating the elastic scattering information from specular reflections. The scattered light 1034′ is collected by a fiber bundle consisting of an array of coherent single mode or multi-mode fibers. The distal tip of the fiber is maintained one focal length away from lens (L2) 1098 to image the angular distribution of scattered light. In the embodiment shown in FIG. 43B, the sample is located in the front focal plane of lens (L2) 1098 using a mechanical mount 1100. In the endoscope compatible probe 1093 shown in FIG. 43A, the sample is located in the front focal plane of lens (L2) 1098 using a transparent sheath 1102.

As illustrated in FIG. 43A, scattered light 1104 emerging from a proximal end 1105 of the fiber bundle (F4) 1094 is recollimated by lens (L4) 1107 and overlapped with the reference beam 1014′ using beam splitter (BS) 1108. The two combined beams 1110 are re-imaged onto the line scan array 1026′ using lens (L5) 1112. The focal length of lens (L5) 1112 may be varied to optimally fill the line scan array 1026′. The line scan array 1026′ passes the detected signal to a processing system, such as a computer 1111, to process the returned scattered signal to determine structural and depth-resolved information about the sample. The resulting optical signal contains information on each scattering angle across the vertical dimension of the slit 1040′ as described above for the apparatus of FIGS. 40 and 41. It is expected that the above-described SS a/LCI system 1012′, as an example, the fiber optic probe can collect the angular distribution over a 0.45 radian range (approximately 30 degrees) and can acquire the complete depth-resolved scattering distribution or combined beams 1110 in a fraction of a second.

There are several possible schemes for creating the fiber probe which are the same from an optical engineering point of view. One possible implementation would be a linear array of single mode fibers in both the signal and reference arms. Alternatively, a reference arm 1096 could be composed of an individual single mode fiber with the signal arm 1082 consisting of either a coherent fiber bundle or linear fiber array.

The probe 1093 can also have several implementations which are substantially equivalent. These would include the use of a drum or ball lens in place of lens (L2) 1098. A side-viewing probe could be created using a combination of a lens and a mirror or prism or through the use of a convex mirror to replace the lens-mirror combination. Finally, the entire probe can be made to rotate radially in order to provide a circumferential scan of the probed area.

Another exemplary embodiment of a fiber optic SS a/LCI system is the illustrated a/LCI system 1010″ in FIG. 43B. In this system 1010″, a swept-source light source 1012″ is used just as in the fiber-optic a/LCI system 1010′ of FIG. 43A. Other components provided in the system 1010″ of FIG. 43B are also included in the system 1010′ of FIG. 43A, which are indicated with common element designations. In the fiber optic SS a/LCI system 1010″, the angular distribution of scattered light from the sample is captured by locating the distal end of the fiber bundle in a conjugate Fourier transform plane of the sample using a collecting lens. This angular distribution is then conveyed to the distal end of the fiber bundle where it is imaged using a 4f system onto the line scan array. A beam splitter is used to overlap the scattered sample field with a reference field prior to the line scan array so that low-coherence interferometry can also be used to obtain depth resolved measurements.

As illustrated in FIG. 43B, light 1011″ is generated by a swept-source light source 1012″. An optical isolator 1113 protects the light source 1012″ from back reflections. The fiber splitter 1080 generates a reference beam 1014″ and a sample beam 1016″. The reference beam 1014″ passes through an optional polarization controller 1114, a length of fiber 1117 (to path optical path lengths), and then to the lens (L4) 1107 to the beam splitter 1108. The sample beam 1016″ travels through a polarization controller 1115 and a fiber polarizer 1116 to improve polarization of source light and align polarization with the axis of the fiber polarizer 1116. The delivery or illumination fiber 1090 is provided to the fiber probe 1093. The lens 1084 captures returned scattered light from the sample 1017, which is collected at a particular angle (or a small range of angles) by the collection fiber bundle 1094. Captured light is carried through the collection fiber bundle 1094 comprised of a plurality of collection fibers 1095. The captured light travels back up the fiber probe 1093 through optical lens (L2) 1098 and lens (L3) 1092. The reference beam 1014″ and returned scattered light from the sample 1017 are mixed at the beam splitter 1108 with the resulting interfering signal 1110 being passed to a line scan array detector 1026′ as previously described. The line scan array 1026′ passes the detected signal to a processing system, such as the computer 1111″, to process the return scattered signal to determine structural and depth-resolved information about the sample. The resulting optical signal contains information on each scattering angle across the vertical dimension of the slit 1040′ as described above for the apparatus of FIGS. 40 and 41. It is expected that for one embodiment of the above-described SS a/LCI system 1010″, as an example, the fiber optic probe 1093 can collect the angular distribution over a 0.45 radian range (approximately 30 degrees) and can acquire the complete depth-resolved scattering distribution or combined beams 1110 in a fraction of a second.

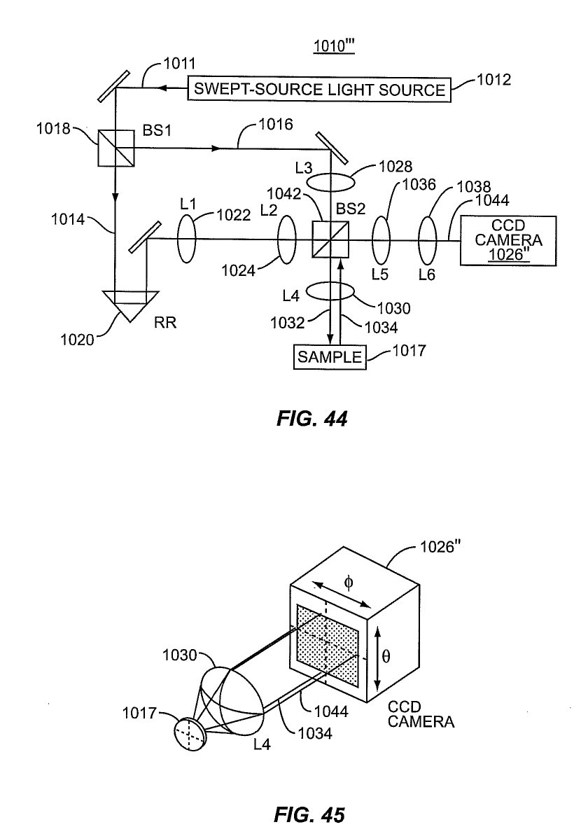

The use of a swept-source light source also opens up the possibility of another system architecture that has the capability to acquire scattering information from more than one scattering plane from a sample. This implementation is referred to as a “Multiple Angle Swept-source a/LCI” system or MA SS a/LCI. An example of an MA SS a/LCI system 1010′″ is illustrated in FIGS. 44 and 45, which has a similar arrangement to the SS a/LCI system 1010 of FIGS. 40 and 41, except that a two-dimensional detection device 1026″ is provided in the form of a CCD camera. This allows acquiring returned scatter information from a sample at multiple angles or range of angles at the same time or essentially at the same time. This arrangement allows one to obtain a larger amount of information with a single measurement compared to one-dimensional approaches. In a one-dimensional scheme, the scattering distribution is acquired across a single line of angles and requires sample manipulation to obtain information in another scattering plane. By acquiring information about the sample from multiple angles or a range of angles, it is possible to achieve better signal-to-noise in the resulting measurements and/or acquire more information about the sample such as the major and minor axis for non-spheriodal scatterers.

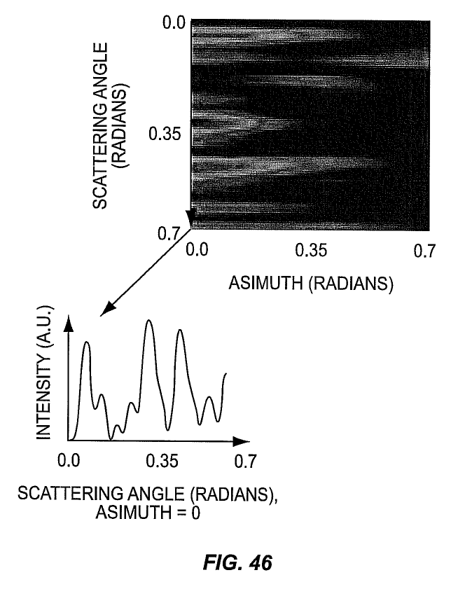

The MA SS a/LCI system 1010′″ is exemplified in FIGS. 44 and 45, and is similar to the SS a/LCI of FIGS. 40 and 41, except that the line scan array 1026 is replaced by a two-dimensional array 1026″, such as a CCD camera. The steps set forth in the flowchart of FIG. 42 are applicable for this embodiment, except that this embodiment will involve the mixed returned scattered light being directed to a two-dimensional detector 1026″ (block 1070) and detecting dispersed light to recover spatially and depth-resolved information about the sample using the two-dimensional detector 1026″ (block 1072). Further, the MA SS a/LCI system 1010′″ can be implemented using a fiber optic probe and bundle detection system like that of FIG. 43B, except that the line scan array 1026′ is replaced by a two-dimensional detector 1026″, namely a CCD camera. In either implementation example, the CCD camera 1026″ may acquire a frame at each step as the swept-source light source 1012, such as a swept-source laser, is swept (or more likely may capture a frame as the light source sweeps continuously resulting in a range of wavelengths captured in each frame). The swept-source light source 1012 sweeps over frequencies as the CCD camera 1026″ synchronously captures images from the combined beams 1044 from the sample 1017. With this method, the acquisition time may decrease to a fraction of a second. The collection of frames from a sweep of the swept-source light source 1012 will then be processed to generate wavelength information for either a range of scattering angles in the θ and φ direction, a set of discrete angles, or some combination of the two. Further processing will provide information about the nature of the scatterers in the sample 1017. FIG. 46 illustrates an exemplary model of a two-dimensional image of a diffraction pattern due to eight micron spheroid distribution using the MA SS a/LCI system of FIG. 44.

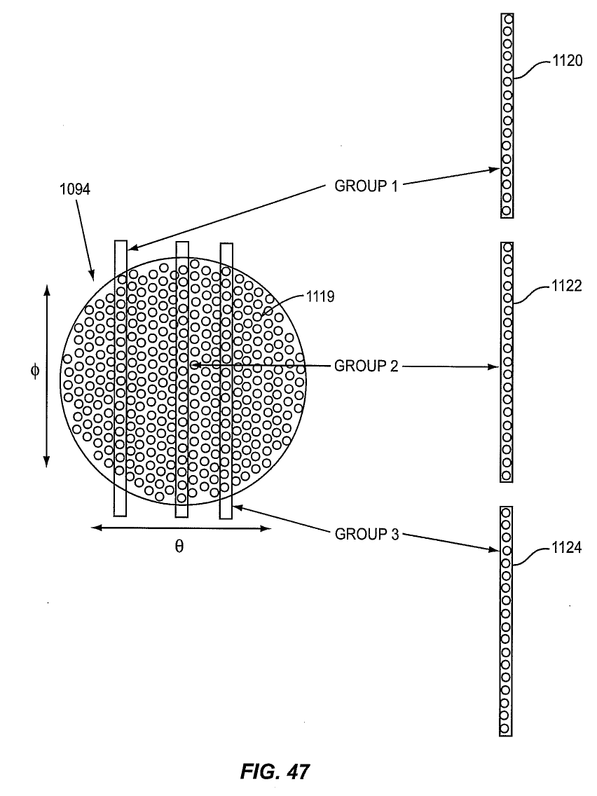

The MA SS a/LCI system 1010′″ may also be implemented using a broadband light source, such as a superluminescent diode (SLD), and using a spectrometer detection device. In either case, whether using a broadband light source or swept-source light source 1012, in the fiber optic embodiment of a MA SS a/LCI system 1010′″, the fiber bundle 1094 that receives the combined beams 1044 from the sample 1017 can be captured by a plurality of optical fibers 1119 in the fiber bundle 1094, as illustrated in FIG. 47. Here, the optical fiber breakout is issued to bring optical fibers 1119 from the fiber bundle 1094 to one or more horizontal lines 1120, 1122, 1124, but radial and circular breakouts are also possible, which are different types of sections of the optical fibers 1119. The number of optical fibers 1119 shown in a vertical row is one optical fiber 1119 wide, but any number is possible. The number of optical fibers 1119 used horizontally at a given position in the vertical column will determine the angular range of the particular reading from a detection device 1026″or spectrometer, as the case may be.

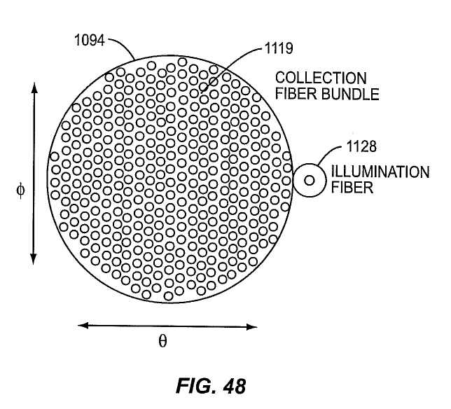

One possible distribution of the scattering angles across the CCD camera 1026″ is shown in FIG. 48. In this implementation, angles in θ are spread vertically and angles in φ are spread horizontally. The angles may or may not be distributed evenly in θ and φ. For example, in the endoscopic implementation described later in this application, an illumination fiber 1128 lies on one side of a fiber bundle and the angles acquired will be determined by the locations of the fibers in the bundle. This is shown in FIG. 48, where the system 1010′″ will be able to collect some subset of the angles in 0 and φ, but even here there may be enough additional information acquired that additional structural measurements can be generated by the data processing.

Potential components for the CCD camera 1026″ include but are not limited to a Cascade:Photometrics™ 650 CCD camera as the image detector. For the light source, the Thorlabs INTUN™ continuously tunable laser is an example of one of many suitable sources. This example would be useful because the center wavelength is 780 nm, which is compatible with standard NIR optical elements, including the Cascade camera, and offers a tuning range of 15 mm, which is comparable to the line width used in SS a/LCI systems previously described. The tuning speed of 30 nm/s for this source is optimal for synchronization with the Cascade CCD camera as better than 0.1 nm resolution can be achieved based on the 300 Hz frame rate which can be realized when using a region of interest with the Cascade CCD. The SS a/LCI scheme will improve acquisition time and upgrade the a/LCI system to a state-of-the-art technology for studies of cell mechanics at faster time scales.

The data acquisition may be limited by the frame rate of the CCD camera 1026″ and not by the sweep speed of the swept-source light source 1012. Table 4 below lists exemplary CCD cameras. The fastest listed is only 1000 frames per second, so if 1000 wavelength points are required, a full scan will take approximately 1 second. It may be possible to scan faster if fewer pixels are needed in this example, or if fewer points in the wavelength can be used. Several of these cameras will let the user target specific regions of interest to acquire images, thus speeding up the frame rate. For example, with the Atmel® camera, if one uses a region of interest that is 100×100 pixels for a total of 10,000 pixels, then the frame rate might be as high as 15,000 frames per second allowing a scan time of 70 milliseconds for 1000 wavelength points. It is expected that the speed of the CCD cameras will increase over time and the increased camera speed will translate into higher performance of the MA SS a/LCI system.

TABLE 4
Examples of High Speed CCD Cameras

| | | | | Readout rate |
|---|---|---|---|---|
| | | | Pixel size | (1000 pixels/ |
| Manufacturer | λ range (nm) | Pixel number | (μm) | second) |
| Atmel | 400-900 | 2000 × 1000 | 5 | 150000 |
| Hamamatsu | 400-950 | 250 × 1024 | 25 | 10000 |
| Fairchild | 400-850 | 512 × 512 | 17 | Up to 1000 |
| Imaging | | | | frame/sec |

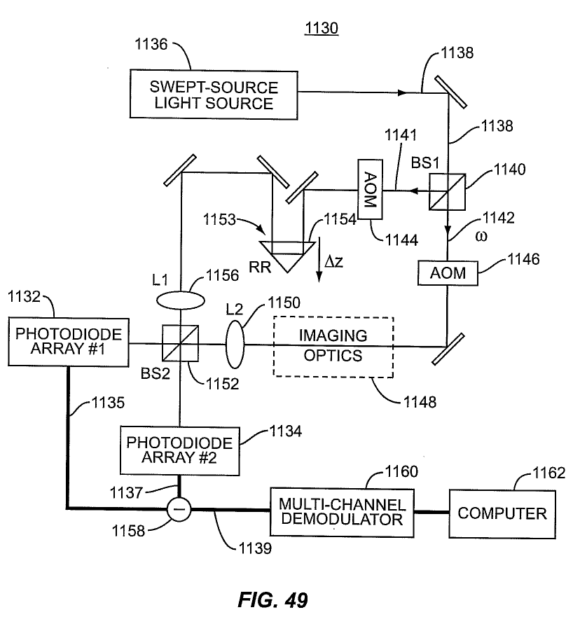

In addition to the SS a/LCI and MA SS a/LCI implementations described herein, a time-domain a/LCI implementation is also possible. An example of this a/LCI system 1130 implementation is shown by example in FIG. 49. This system 1130 physically scans the depth of a sample, but uses an array of detectors to simultaneously collect returned scattered light from the sample from multiple angles at the same time or essentially the same time. This allows the system 1130 to simultaneously collect light from multiple angles increasing throughput by a factor equal to the number of angle acquisitions.

The system 1130 uses photodiode arrays #1 and #2 1132, 1134 to collect angular scattered light from the sample (not shown). The system 1130 provides a swept-source light source 1136 in the form of a Ti:Sapphire laser operating in a pulsed mode in this embodiment. The swept-source light source 1136 directs light 1138 to a beam splitter (BS1) 1140, which splits the light 1138 into a reference signal 1141 and sample signal 1142. The reference signal 1141 goes through acousto optic modulator (AOM) 1144 with w+10 MHz, and then through retroreflector (RR) 1154 mounted on a reference arm 1153, wherein the retroreflector (RR) 1154 is moved by a distance, δz to change the depth in the sample to perform depth scans. The sample signal 1142 goes through AOM 1146 with frequency ‘ω’ and then through imaging optics 1148. Imaging optics 1148 shine collimated light onto the sample and then collects the angular scattered light from the sample. The reference signal 1141 and the angular scattered light are combined at beamsplitter (BS2) 1152 and then imaged onto the photodiode arrays #1 and #2 1132, 1134. Signals 1135, 1137 from each photodiode 1132 or 1134 are subtracted from the photodiode in the other array 1132 or 1134 which corresponds to the same angular location. A multi-channel demodulator 1160 is used on a subtracted signal 1139. All signals then go to a computer 1162 for processing. Processing of the time-domain depth information from the subtracted signal 1139 and received by the multi-channel demodulator 1160 can be performed just as previously described in above for this embodiment, as possible examples or methods.

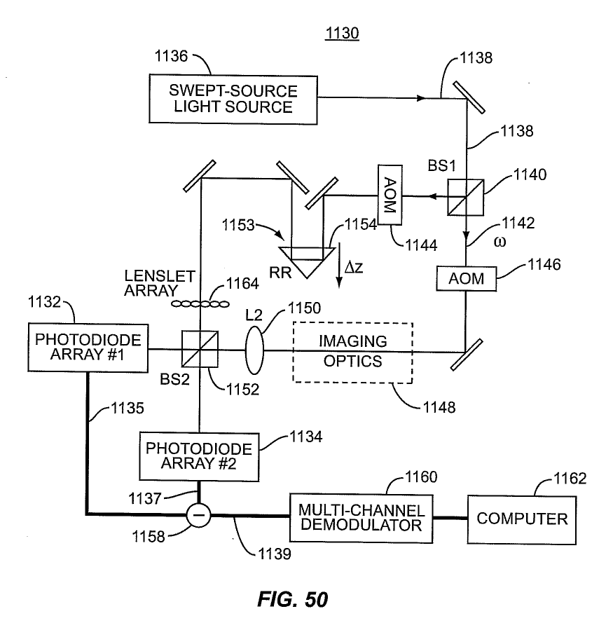

FIG. 50 illustrates the same system 1130 of FIG. 49, except that lens L1 1156 is changed out for lenslet array 1164. Each lenslet in the lenslet array 1164 provides the reference arm 1153 for one angular position. A lenslet array can be used for each angular position in the photodiode arrays 1132, 1134 to properly capture angular scattered light from the sample.

Even though the systems 1130 illustrated in FIGS. 49 and 50 obtain depth-resolved information regarding tissue in the time domain, these systems 1130 are still capable of examining and monitoring tissue during the course of the same or concomitant medical procedure to determine if a therapeutic should be applied to the tissue. For example, in a typical setup, data about the sample may be acquired at 20 to 60 angles and takes approximately 6 minutes for a 60 angle scan. However, the implementation in FIG. 50 should be able to acquire this same data set in at least six (6) seconds to feedback information regarding the tissue. While still possibly slower than Fourier domain techniques (due to the higher intrinsic signal-to-noise ratio available in the Fourier domain systems), this can be an improvement in speed and be used for many applications. This implementation calls for photodiode arrays that can acquire enough line scans, such that there are up to 500 in a depth scan. If a scan takes six (6) seconds, this is approximately 100 per second, which is much less than the line rates of any of the cameras listed in Table 1. Given that cameras can capture frames much faster than this, the limit to acquisition speed may be the amount of available light scattered from the sample.

Note that this system uses some means of subtracting the signals 1135, 1137 on the photodiodes arrays 1132, 1134 on a photodiode basis and then demodulating each channel. This may be accomplished in a serial or parallel fashion. One implementation would be to digitally acquire data from the photodiode arrays (as in the case of a line scan camera) and then use a digital signal processor (DSP) chip or similar to subtract and demodulate the data. This may require that the offset frequency between the two AOMs be less than the line rate of the line scan arrays. Since line scan arrays that receive signal data up to 100,000 lines/second exist, an offset of <50 KHz may be acceptable.

A second implementation would be to use the photodiode arrays 1132, 1134 and perform the subtraction in an analog basis. It may be the case that the two photodiode arrays are actually two sections of the same two-dimensional array. There also may then be a dedicated demodulator for each photodiode pair or, again, a digitizer and appropriate digital signal processor (DSP) chips.

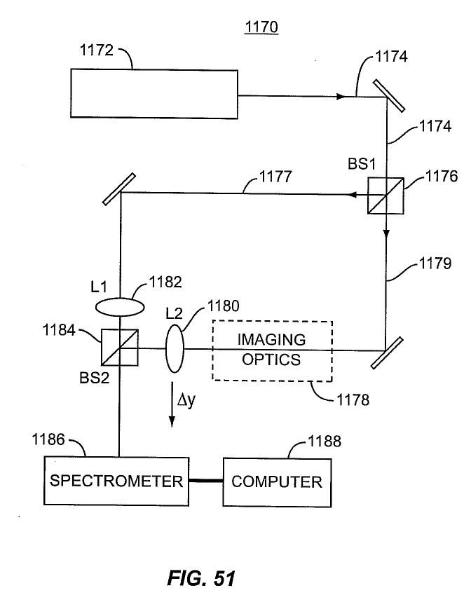

In another embodiment and approach to collecting information about a sample of interest, a step forward from time domain a/LCI systems is taken to still collect the angular information in a serial fashion. However, depth information is collected from a sample of interest using a Fourier domain approach. The light source that may be used can include a broadband light source in combination with a spectrometer to process spectrally-resolved information about the sample. Alternatively, a swept-source light source with a photodiode or another implementation may be used. FIG. 51 shows an implementation of such a system 1170. The system 1170 illustrated employs a Ti:Sapphire pulsed laser light source 1172 for a broadband light source with a single line spectrometer 1186 in place of a photodiode for signal collection. In FIG. 51, the laser 1172 in a pulsed mode generates light 1174. Beam splitter (BS1) 1176 splits the light 1174 into a reference signal 1177 and a sample signal 1179. The reference signal 1177 travels through optic(s), lens (L1) 1182, while the sample signal 1179 travels through imaging optics 1178, which illuminate a sample (not shown) and capture scattered light returned from the sample. Lens (L2) 1180 is moved to set the particular angle of scattered light from the sample that is being viewed by the spectrometer 1186. Beamsplitter (BS2) 1184 combines the reference signal 1177 and the sample signal 1179 which then travels to spectrometer 1186. The combined signal then passes through computer 1188 for processing. The spectrometer 1186 captures at least one line of returned scattered light from the sample. The spectrometer 1186 could capture more than one line (i.e., it could be an imaging spectrometer) to create a system that is closer to the current working implementation. This could be advantageous to either use a spectrometer with fewer lines, or allow capture of a larger angular range (or finer resolution).

Since this system 1170 does not use a time domain data acquisition approach, the AOMs 1144, 1146 and the moving retroreflector (RR) 1154 in the reference arm 1153, as provided in the systems 1130 in FIGS. 49 and 50, are not needed. This system 1170 shows one spectrometer 1186, but it is possible to use a second spectrometer on the other port of the beam splitter for additional signal for potential increases in optical signal-to-noise ratio (OSNR) or advanced processing or other reasons. This implementation has a significant OSNR advantage, on the order of the number of pixels covered by the broadband light source in the spectrometer 1186. As noted, this system 1170 can also be implemented with a swept-source light source in place of the Ti:Sapphire laser, and a single photodiode in place of the spectrometer 1186.

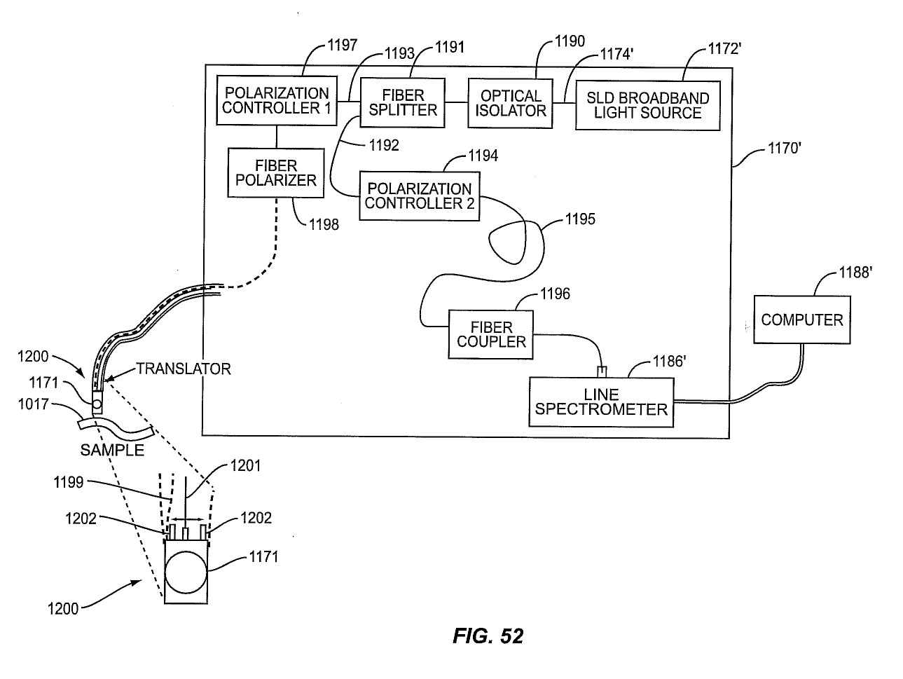

FIG. 52 illustrates another implementation of the Fourier domain system 1170 of FIG. 51, with serial detection of angles, but using a fiber-optic approach. The angular information from the sample is collected serially by moving a fiber (or more than one fiber) back and forth in front of lens 1171, which collects the returned angular scattered light from the sample 1017. The optical engine is almost entirely fiber-optic in this particular implementation with the free space optics provided inside a line spectrometer 1186′. This implementation is beneficial in terms of cost and ease of construction, since optical fibers are usually cheaper and easily to deal with than free space optical systems.

As illustrated in FIG. 52, light 1174′ is generated by SLD broadband light source 1172′. An optical isolator 1190 protects the light source 1172′ from back reflections. A fiber splitter 1191 generates a sample signal 1193 and a reference signal 1192. The reference signal 1192 passes through an optional polarization controller 1194, a length of fiber 1195 (to path optical path lengths), and then to a fiber coupler 1196 (i.e., a fiber splitter used in opposite direction). The sample signal 1193 travels through a polarization controller 1197 and a fiber polarizer 1198 to improve polarization of source light and align polarization with the axis of the fiber polarizer 1198. An illumination fiber 1199 is provided to a fiber probe 1200 and passes through lens 1171 to illuminate the illumination fiber 1199. Lens 1171 captures returned scattered light from the sample 1017, which is collected at a particular angle (or at a small range of angles) by a collection fiber 1201. The collection fiber 1201 is moved to capture information from different angles from the sample 1017. A motion mechanism shown is based on electromagnets 1202 in this embodiment. Any method to move the collection fiber 1201 with respect to the sample 1017 can be used. The collection fiber 1201 can be moved in one dimension or in multiple dimensions. Light from the collection fiber 1201 travels back up the fiber probe 1200 and into an optical engine (not shown) where it connects to the fiber coupler 1196. The reference signal 1193 and returned scattered light from the sample 1017 are mixed at the fiber coupler 1196 with the resulting light signal passed to the line spectrometer 1186′. The combined signal then passes through computer 1188 for processing. Again, this embodiment is illustrated with one collection fiber, but it could be implemented with multiple collection fibers that are moved to either reduce the needed size of the spectrometer or increase the angular range.

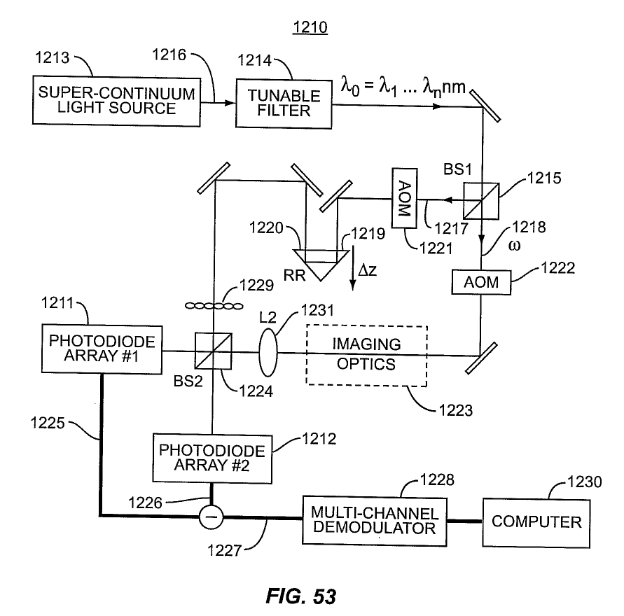

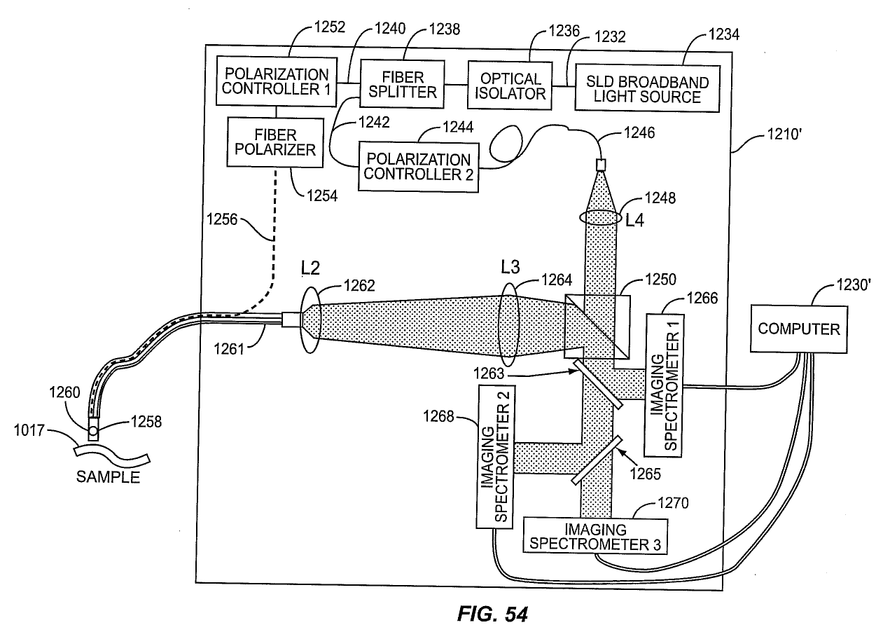

Another implementation of a/LCI is a multi-spectral a/LCI system. Embodiments of multi-spectral a/LCI systems 1210, 1210′ are illustrated in FIGS. 53 and 54. In this approach, a/LCI measurements are performed at multiple wavelengths (or frequencies) that may be separated, such as by a few up to hundreds of nanometers. The system 1210 responds like an f/LCI system, where depth information regarding a sample of interest is obtained at multiple wavelengths. Multi-spectral a/LCI can obtain both depth and angular information at multiple wavelengths. This system 1210 can thereafter generate the structural and depth information using techniques that utilize a/LCI or f/LCI. Alternatively, the system 1210 can be used to measure tissue responses at a few wavelengths to determine properties of blood, water or other characteristics of the tissue.

The system 1210 of FIG. 53 uses time domain for obtaining depth information and involves parallel acquisition of angular information and a tunable source for multi-spectral information acquisition. The system 1210 uses photodiode arrays #1 and #2 1211, 1212 to collect angular scattered light from the sample (not shown). The system 1210 provides a super-continuum light source 1213 with a tunable filter 1214 that provides a 10 to 20 nm spectral bandwidth and that can be tuned over a few up to hundreds of nanometers in this example. A commercially available example of this light source is the SC450-AOTF from Fianium®, which combines a fiber-optic super-continuum light source with an acousto-optic tunable filter. Other source examples could include white light sources, such as Xenon lamps as an example. Other filters may be used, including but not limited to liquid crystal (LC) optical filters.

The super-continuum light source 1213 directs light 1212 to a beam splitter (BS1) 1215, which splits the light 1216 into a reference signal 1217 and sample signal 1218. The reference signal 1217 goes through AOM 1221, and then through retroreflector (RR) 1219 mounted on a reference arm 1220, wherein the retroreflector (RR) 1219 is moved by the reference arm 1220 to change the depth in the sample to perform depth scans. The sample signal 1218 goes through AOM 1222 with frequency ‘ω’ and then through imaging optics 1223. Imaging optics 1223 shine light from the super-continuum light source 1213 onto a sample and then collects the angular scattered light from the sample. The reference signal 1217 and the angular scattered light are combined at beamsplitter (BS2) 1224 and then imaged onto the photodiode arrays #1 and #2 1211, 1212. Signals 1225, 1226 from each photodiode array 1211 or 1212 are subtracted from the photodiode in the other array 1211 or 1212 which corresponds to the same angular location. A multi-channel demodulator 1228 is used on the resulting subtracted signal 1227. The subtracted signal 1227 travels to a computer 1230 for processing.

Another approach to the multi-spectral a/LCI system 1210 in FIG. 53 is to use a broadband light source with multiple spectrometers. An example of one such system 1210′ is illustrated in FIG. 54. The system 1210′ uses Fourier domain for obtaining depth information about a sample, and parallel acquisition of angular information and parallel acquisition of multi-spectral information by use of broadband filters and multiple spectrometers. The optical engine is almost entirely fiber-optic in this particular implementation with the free space optics provided inside imaging spectrometers 1266, 1268, 1270. This implementation is beneficial in terms of cost and ease of construction, since optical fibers are usually cheaper and easily to deal with than free space optical systems.

As illustrated in FIG. 54, light 1232 is generated by a SLD broadband light source 1234. An optical isolator 1236 protects the light source 1234 from back reflections. A fiber splitter 1238 generates a sample signal 1240 and a reference signal 1242. The reference signal 1242 passes through an optional polarization controller 1244, a length of fiber 1246 (to path optical path lengths), and then to a lens (L4) 1248 to a beamsplitter 1250. The sample signal 1240 travels through a polarization controller 1252 and a fiber polarizer 1254 to improve polarization of source light and align polarization with the axis of the fiber polarizer 1254. An illumination fiber 1256 is provided to a fiber probe 1258 and passes through lens 1260 to illuminate the illumination fiber 1256. The lens 1260 captures returned scattered light from the sample 1017, which is collected at a particular angle (or a small range of angles) by a collection fiber 1261. Captured light carried through the collection fiber 1261 travels back up the fiber probe 1258 through optical lens (L2) 1262 and lens (L3) 1264. The reference signal 1242 and returned scattered light from the sample 1017 are mixed at beamsplitter 1250. Two free space optical filters 1263, 1265 split the scattered light spectrum from the sample into three light signals, each being provided to a separate imaging spectrometer 1266, 1268, 1270. This allows the spectrally-resolved scattered light from the sample 1017 to be processed by computer 1230′ using Fourier domain techniques to obtain structural and depth information about the sample.

It is possible to provide this system 1210′ with one spectrometer, although the combination of multiple spectrometers allows for high spectral resolution for the Fourier domain depth detection and the broad range of wavelengths needed to acquire the multi-spectral information. The system 1210′ can be expanded to as many sections of the optical spectrum as needed. Fiber implementations based on fiber couplers and fiber filters are also possible.

The system 1210′ may also be provided with a broadband swept-source light source for the acquisition of depth information and the acquisition of multi-spectral information. Another approach is to multiplex together multiple sources at different wavelengths to obtain the multi-spectral information. For example, an 830 nm center wavelength, 20 nm 3 dB width SLD could be multiplexed together with a 650 nm center wavelength, 15 nm 3 dB width SLD to obtain a/LCI information at two wavelengths. Further, as the various wavelengths become farther apart, it may be necessary to put in compensation components to account for the variation in index of refraction at the different wavelengths. For example, if one is using a 400 nm and an 800 nm wavelength, it may be the case that when the interferometer arms are path length matching for the 400 nm wavelength, they are mismatched for the 800 m wavelength by more than the imaging depth available with the spectrometer (typically 1 to 2 mm).

The f/a/LCI systems and methods described herein can be clinically viable methods for assessing tissue health without the need for tissue extraction via biopsy or subsequent histopathological evaluation. The f/a/LCI systems and methods described herein can be applied for a number of purposes: for example, early detection and screening for dysplastic tissues, disease staging, monitoring of therapeutic action, and guiding the clinician to biopsy or surgery sites. The non-invasive, non-ionizing nature of the optical biopsy based on an f/a/LCI probe means that it can be applied frequently without adverse affect. The potential of f/a/LCI to provide rapid results will greatly enhance its widespread applicability for disease screening.

Nuclear morphology measurement is also possible using the f/a/LCI systems and methods described herein. Nuclear morphology is a necessary junction between a cell's topographical environment and its gene expression. One application of the f/a/LCI systems and methods is to connect topographical cues to stem cell function by investigating nuclear morphology. In one embodiment, the f/a/LCI systems and methods use a swept-source light source approach described herein and create and implement light scattering models. The second is to provide nuclear morphology as a function of nanotopography. Finally, by connecting nuclear morphology with gene expression, the structure-function relationship of stem cells, e.g., human mesenchymal stem cells (hMSC), under the influence of nanotopographic cues can be established.

The f/a/LCI methods, processes, techniques, and systems described herein can also be used for cell biology applications and medical treatment based on such applications. Accurate measurements of nuclear deformation, i.e., structural changes of the nucleus in response to environmental stimuli, are important for signal transduction studies. Traditionally, these measurements require labeling and imaging, and then nuclear measurement using image analysis. This approach is time-consuming, invasive, and unavoidably perturbs cellular systems. The f/a/LCI techniques described herein offer an alternative for probing physical characteristics of living systems. The f/a/LCI techniques disclosed herein can be used to quantify nuclear morphology for early cancer detection, diagnosis and treatment, as well as for noninvasively measuring small changes in nuclear morphology in response to environmental stimuli. With the f/a/LCI methods, processes, techniques, and systems provided herein, high-throughput measurements and probing aspherical nuclei can be accomplished. This is demonstrated for both cell and tissue engineering research. Structural changes in cell nuclei or mitochondria due to subtle environmental stimuli, including substrate topography and osmotic pressure, are profiled rapidly without disrupting the cells or introducing artifacts associated with traditional measurements. Accuracy of better than 3% can be obtained over a range of nuclear geometries, with the greatest deviations occurring for the more complex geometries.

In one embodiment disclosed herein, the f/a/LCI systems and methods described herein are used to assess nuclear deformation due to osmotic pressure. Cells are seeded at high density in chambered coverglasses and equilibrated with 500, 400 and 330 mOsm saline solution, in that order. Nuclear diameters are measured in micrometers to obtain the mean value +/− the standard error within a 95% confidence interval. Changes in nuclear size are detected as a function of osmotic pressure, indicating that the f/a/LCI systems and methods disclosed herein can be used to detect cellular changes in response to factors which affect cell environment. One skilled in the art would recognize that many biochemical and physiological factors can affect cell environment, including disease, exposure to therapeutic agents, and environmental stresses.

To assess nuclear changes in response to nanotopography, cells are grown on nanopatterned substrates which create an elongation of the cells along the axis of the finely ruled pattern. The f/a/LCI systems and processes disclosed herein are applied to measure the major and minor axes of the oriented spheroidal scatterers in micrometers through repeated measurements with varying orientation and polarization. A full characterization of the cell nuclei is achieved, and both the major axis and minor axis of the nuclei is determined, yielding an aspect ratio (ratio of minor to major axes).

The f/a/LCI systems and methods disclosed herein can also be used for monitoring therapy. In this regard, the f/a/LCI systems and methods are used to assess nuclear morphology and subcellular structure within cells (e.g., mitochondria) at several time points following treatment with chemotherapeutic agents. The light scattering signal reveals a change in the organization of subcellular structures that is interpreted using a fractal dimension formalism. The fractal dimension of sub-cellular structures in cells treated with paclitaxel and doxorubicin is observed to increase significantly compared to that of control cells. The fractal dimension will vary with time upon exposure to therapeutic agents, e.g., paclitaxel, doxorubicin and the like, demonstrating that structural changes associated with apoptosis are occurring. Using T-matrix theory-based light scattering analysis and an inverse light scattering algorithm, the size and shape of cell nuclei and mitochondria are determined. Using the f/a/LCI systems and methods disclosed herein, changes in sub-cellular structure (e.g., mitochondria) and nuclear substructure, including changes caused by apoptosis, can be detected. Accordingly, the f/a/LCI systems and processes described herein have utility in detecting early apoptotic events for both clinical and basic science applications.

Although embodiments disclosed herein have been illustrated and described herein with reference to preferred embodiments and specific examples thereof, it will be readily apparent to those of ordinary skill in the art that other embodiments and examples can perform similar functions and/or achieve like results. The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. All such equivalent embodiments and examples are within the spirit and scope of the present invention and are intended to be covered by the appended claims.

It will also be apparent to those skilled in the art that various modifications and variations can be made to the present invention without departing from the spirit and scope of the invention. Thus, the disclosure is not intended to be limited to the examples and designs described herein, but is to be accorded the widest scope consistent with the principles and novel features disclosed herein. For example, the present invention is not limited to a particular Fourier domain or angle-resolved optical biopsy system, tissue type examined, therapy or therapeutic, an endoscope or endoscopic probe, control systems or interfaces, or methods, processes, techniques disclosed herein and their order.

The embodiments set forth above represent the necessary information to enable those skilled in the art to practice the invention and illustrate the best mode of practicing the invention. Upon reading the following description in light if the accompanying drawings figures, those skilled in the art will understand the concepts of the invention and will recognize applications of these concepts not particularly addressed herein. It should be understood that these concepts and applications fall within the scope of the disclosure and the claims that follow.

We claim:

1. A method for examining and monitoring tissue to determine if a therapeutic should be applied to the tissue, comprising during the same or concomitant procedure:
(a) optically examining a tissue to detect anomalies in the tissue generally not perceptible to the human eye employing a real-time f/a/LCI system;
(b) monitoring feedback information regarding the examination of the tissue from the real-time f/a/LCI system;
(c) determining if a treatment should be applied to the tissue based on the feedback information; and
(d) applying a therapy on the tissue if treatment is determined to be applied based on the feedback information.

2. The method of claim 1, wherein the real-time f/a/LCI system is a system comprised from the group consisting of a Fourier domain low coherence interferometer (LCI) (fLCI), an angle-resolved LCI (a/LCI), a Fourier domain, angled-resolved LCI (faLCI), a swept-source (SS) a/LCI (SS a/LCI), a multiple angle (MA) SS a/LCI (MA SS a/LCI), a multiple channel time domain a/LCI, and a multi-spectral a/LCI.

3. The method of claim 1, wherein an optical probe of the real-time f/a/LCI system is integrated in a channel of an endoscopic probe used to optically examine the tissue.

4. The method of claim 1, wherein the feedback information is provided by the real-time f/a/LCI system in approximately one (1) second or less after optically examining the tissue.

5. The method of claim 1, further comprising controlling the real-time f/a/LCI system during optically examining the tissue via computer control having a user interface.

6. The method of claim 1, further comprising repeating steps (a)-(c) after performing step (c).

7. The method of claim 6, wherein the repeating of steps (a)-(c) is performed during the same or concomitant procedure.

8. The method of claim 6, wherein the repeating of steps (a)-(c) is performed during a subsequent procedure.

9. The method of claim 6, further comprising repeating step (d) after the repeating of steps (a)-(c).

10. The method of claim 9, wherein the repeating of step (d) is performed during the same or concomitant procedure.

11. The method of claim 9, wherein the repeating of step (d) is performed during a subsequent procedure.

12. The method of claim 1, wherein the monitoring is performed by a computer in an automated fashion.

13. The method of claim 1, wherein the therapy applied to the tissue is a therapy comprised from the group consisting of one or more of an applied substance therapeutic, a heat application therapeutic, a cold application therapeutic, a radiation ablation therapeutic, a light ablation therapeutic, radio frequency (RF) ablation, a photodynamic therapy, and tissue removal.

14. The method of claim 1, wherein applying the therapy on the tissue if treatment is determined to be applied based on the feedback information is applied via a therapeutic dispenser.

15. A system for examining and monitoring tissue, and applying a therapy to the tissue, if needed during the same or concomitant procedure, comprising:
a real-time f/a/LCI system adapted to optically examine tissue during the procedure, comprising:
an optical probe that receives light from the tissue in response to a sample beam directed to the tissue;
a detector that detects the received light from the tissue; and
a processor that processes the detected light in real-time to determine information about the tissue not generally perceptible to the human eye and configured to provide feedback information regarding the tissue; and
a therapeutic applicator adapted to apply a therapy to the tissue if treatment is determined to be applied based on the feedback information during the procedure.

16. The system of claim 15, further comprising a processing system adapted to receive the feedback information to determine if a treatment should be applied to the tissue during the procedure.

17. The system of claim 15, wherein the optical probe is employed in an endoscopic probe of an endoscope used to examine the tissue.

18. The system of claim 17, wherein the optical probe is integrated into the endoscopic probe.

19. The system of claim 17, wherein the endoscopic probe comprises an instrument channel configured to receive the optical probe.

20. The system of claim 19, wherein the instrument channel is configured to receive a therapeutic applicator after the tissue is examined and the optical probe is removed from the instrument channel.

21. The system of claim 19, wherein the endoscopic probe further comprises a second instrument channel configured to receive a therapeutic applicator.

22. The system of claim 19, wherein the optical probe is comprised of optical fiber.

23. The system of claim 22, wherein the optical fiber carries a light signal to the tissue for introducing light for examination of the tissue by an eyepiece of the endoscopic probe and for directing the sample beam to the tissue.

24. The system of claim 23, wherein the optical fiber is configured to provide a light therapeutic to the tissue.

25. The system of claim 17, further comprising a probe tip adapted to surround a distal end of the endoscopic probe for protecting the endoscopic probe and the optical probe during application.

26. The system of claim 15, wherein the real-time f/a/LCI system is a system comprised from the group consisting of a Fourier domain low coherence interferometer (LCI) (fLCI), an angle-resolved LCI (a/LCI), a Fourier domain, angled-resolved LCI (faLCI), a swept-source (SS) a/LCI (SS a/LCI), a multiple angle (MA) SS a/LCI (MA SS a/LCI), a multiple channel time domain a/LCI, and a multi-spectral a/LCI.

27. A method of treating a patient having precancerous, cancerous, or diseased tissue, comprising, during the same or concomitant procedure:
(a) optically examining the patient's tissue to detect anomalies in the tissue generally not perceptible to the human eye employing a real-time f/a/LCI system;
(b) monitoring feedback information regarding the examination of the tissue from the real-time f/a/LCI system;
(c) determining if a treatment should be applied to the tissue based on the feedback information; and
(d) applying a therapy on the tissue if treatment is determined to be applied based on the feedback information.

28. The method of claim 27, wherein the real-time f/a/LCI system is a system comprised from the group consisting of a Fourier domain low coherence interferometer (LCI) (fLCI), an angle-resolved LCI (a/LCI), a Fourier domain, angled-resolved LCI (faLCI), a swept-source (SS) a/LCI (SS a/LCI), a multiple angle (MA) SS a/LCI (MA SS a/LCI), a multiple channel time domain a/LCI, and a multi-spectral a/LCI.

29. The method of claim 28, wherein the repeating of steps (a)-(c) is performed during the same or concomitant procedure.

30. The method of claim 28, wherein the repeating of steps (a)-(c) is performed during a subsequent procedure.

31. The method of claim 28, further comprising repeating step (d) after the repeating of steps (a)-(c).

32. The method of claim 31, wherein the repeating of step (d) is performed during the same or concomitant procedure.

33. The method of claim 31, wherein the repeating of step (d) is performed during a subsequent procedure.

34. The method of claim 27, wherein the therapy applied to the tissue is a therapy comprised from the group consisting of one or more of an applied substance therapeutic, a heat application therapeutic, a cold application therapeutic, a radiation ablation therapeutic, a light ablation therapeutic, radio frequency (RF) ablation, a photodynamic therapy, and tissue removal.

35. The method of claim 27, wherein applying the therapy on the tissue if treatment is determined to be applied based on the feedback information is applied via a therapeutic dispenser.

36. A method for performing and monitoring an esophageal endoscopy in a patient, comprising during the same or concomitant procedure:
(a) optically examining the esophagus to detect anomalies generally not perceptible to the human eye employing a real-time f/a/LCI system;
(b) monitoring feedback information regarding the examination of the esophagus from the real-time f/a/LCI system;
(c) determining if a treatment should be applied to the examined portion of the esophagus based on the feedback information; and
(d) applying radio frequency (RF) ablation on the examined portion of the esophagus if treatment is determined to be applied based on the feedback information.
